(19) United States
(12) Patent Application Publication
(10) Pub. No.: US 2026/0049061 A1
(43) Pub. Date: Feb. 19, 2026

(54) SMALL MOLECULES AS KCNQ2/3 (KV7.2/3) CHANNEL ACTIVATORS AND THEIR MEDICAL USE THEREOF

(71) Applicant: Humanwell Pharmaceutical US, Inc., Ballwin, MO (US)

(72) Inventors: Xiaozheng Dou, Ballwin, MO (US); Shivansh Kaushik, Ballwin, MO (US); Shuo Zhao, Ballwin, MO (US); Haizhou Sun, Ballwin, MO (US); Subo Liao, Ballwin, MO (US); Jun Yang, Ballwin, MO (US); Hao Zhou, Yichang (CN); Rong Liu, Yichang (CN); Zejian Ding, Yichang (CN); Xiongjun Mou, Yichang (CN)

(21) Appl. No.: 19/078,833

(22) Filed: Mar. 13, 2025

Related U.S. Application Data
(60) Provisional application No. 63/566,668, filed on Mar. 18, 2024.
(60) Provisional application No. 63/682,521, filed on Aug. 13, 2024.

(51) Int. Cl. C07D 235/30; A61K 31/4184; A61K 31/4188; A61K 31/427; A61K 31/437; A61K 31/439; A61K 31/444; A61K 31/4985; A61K 31/55; A61P 25/08; A61P 29/00; C07D 403/12; C07D 417/12; C07D 471/04; C07D 471/18; C07D 487/04; C07D 491/048

(52) U.S. Cl. CPC C07D 235/30; A61K 31/4184; A61K 31/4188; A61K 31/427; A61K 31/437; A61K 31/439; A61K 31/444; A61K 31/4985; A61K 31/55; A61P 25/08; A61P 29/00; C07D 403/12; C07D 417/12; C07D 471/04; C07D 471/18; C07D 487/04; C07D 491/048

(57) ABSTRACT

The present invention relates to a novel class of bicyclic derivatives that function as small-molecule KCQN2/3 (Kv7.2/3) potassium channel activators, along with their pharmaceutical composition, method of preparation, and therapeutic applications. These compounds, represented by Formula (I), and defined by specific substituents and structural characteristics as described in the specification. By modulating or enhancing the activation of Kv7.2/3 potassium channels, these derivatives hold therapeutic potential for treating seizure disorders and other conditions influenced by potassium ion channel modulation.

### CROSS-REFERENCE TO RELATED APPLICATIONS

The present application claims the benefit of U.S. Provisional Application 63/566,668, titled “NOVEL SMALL MOLECULES AS KCNQ2/3 (KV7.2/3) CHANNEL ACTIVATORS AND THEIR MEDICAL USE THEREOF”, filed on Mar. 18, 2024, and U.S. Provisional Application 63/682,521, titled “SMALL MOLECULES AS KCNQ2/3 (KV7.2/3) CHANNEL ACTIVATORS AND THEIR MEDICAL USE THEREOF”, filed on Aug. 13, 2024, both of which are hereby incorporated by reference in their entirety.

### FIELD OF THE INVENTION

The present disclosure generally relates to the novel KCNQ2/3 (Kv7.2/3) channel activators useful in treating medical disorders.

### BACKGROUND OF THE INVENTION

Kv7 subfamily of potassium ion channels have five members and encoded by KCNQ-1 to KCNQ-5 genes respectively; all these five members are voltage-gated ion channels and play a crucial role in membrane excitability of many types of cells. Kv7 channels can assemble as a homomeric tetramer of identical subunits or heteromeric tetramers of certain subunit combination (Delmas, P., Brown, D. A., “pathways modulation neural KCNQ/M(Kv7) potassium channels” Nature Reviews Neuroscience, 2005, 6, 850). Each Kv7 subunit is composed of six transmembrane segments (S1-S6) and cytoplasmic N- and C-terminals; the segments S1, S2, S3 and S4 form the voltage sensing domain (VSD) while segments S5 and S6 and interconnecting loops get involved in the formation of pore domain (PD). The featured long intracellular C-terminus in Kv7 channel contains domains necessary for tetramerization and are involved in the binding and transduction activities of key regulators, such as phosphatidylinositol 4,5-bisphosphate (PIP2), Calmodulin (CaM), synapsin, A-kinase-ankyrin and protein kinase C, and ankyrin-G (Barrese, V., Stott, J. B., Greenwood, I. A., “KCNQ-encoded potassium channels as therapeutic targets” Annu. Rev. Pharmacol. Toxicol. 2018, 6(58), 625-648)

Kv7 channels have attracted much attention due to their association with multiple diseases. In particular, the Kv7.2/3 heteromeric tetramer, as the most abundant Kv7.2 assembly in neocortex and hippocampus, has been identified to be closely associated with epilepsy, pain and other CNS diseases because of their critical contribution to M-current and the maintenance of the resting membrane potentials in neurons. Since the activation of Kv7.2/3 channels can function as a brake to firing in neurons, activators of Kv7.2/3 channels can have great potential to treat CNS diseases like epilepsy and pain (Jepps, T. A., Barrese, V., and Miceli, F. “Editorial: Kv7 Channels: Structure, Physiology, and Pharmacology” Frontiers in Physiology, 2021, 12, Article 679317).

Searching for Kv7 channel openers started in early 1980s; the first selective Kv7 channel activator, retigabine, was approved in EU in 2011 to treat epilepsy (Trobalt) and in US in 2010 as add-on treatment (Potiga) of partial seizures in adults (Stafstrom, C. E., Crippon, S., Kirkpatrick, P. “Ezogabine (retigabine)” Nature Review Drug Discovery, 2011, 10, 729; Jankovic, S., and Llickovic, I. “the preclinical discovery and development of ezogabine for the treatment of epilepsy” Exper. Opin. Drug Discov. 2013, 8(11), 1). This compound was originally discovered by ASTA Medica Gmbh (DE4200259) and co-developed by Valeant and GSK; however, it was discontinued in June 2017 due to the blue-skin side effect caused by pigmentation of retigabine's metabolite dimer (Clark, S., Antell, A., Kaufman, K. “new antiepileptic medication linked to blue discoloration of the skin and eyes” Ther. Adv. Drug. Safety. 2015, 6(1) 15). In order to address the side effects, many different molecules were designed based on molecular structure of retigabine and evaluated in preclinical and clinical studies (Borgini, M., Mondal, P., Liu, R., and Wipf, P. “Chemical modulation of Kv7 potassium channels” RSC Med. Chem. 2021, 12, 483; Suru, A. S. et al., “Flupirtine and retigabine as templates for ligand-based drug design of Kv7.2/3 activators” Org. Biomol. Chem., 2019, 17, 4512-4522). Among them, XEN1101 developed by Xenon was advanced into Phase III clinical trials to treat epilepsy and seizure (U.S. Ser. No. 11/135,214); BHV-7000 developed by Biohaven was advanced into Phase II clinical trials to treat epilepsy (WO2023133256); Pynegabine developed by Haikou Pharmaceuticals/Shanghai Institute of Materia Medica was advanced to Phase I clinical trials for epilepsy treatment (Zhang, Y.-M., et al., J. Med. Chem, 2021, 64, 5816, WO20151653521) and CB03-154 developed by Zhimeng Biopharma was also advanced to Phase I clinical trials for epilepsy treatment (WO2022028548). Several other companies and universities also work on Kv7.2/3 channel activators in order to identify novel molecules for potential medical uses (WO2023125935, WO2019183148, CN108863893, WO2023025276).

What we disclosed here are novel small molecules Kv7.2/3 activators and their use for the treatment of epilepsy, pain and other variety of medical conditions.

### SUMMARY OF THE INVENTION

The following is an overview of the subject matter described in detail in this invention. This summary is not intended to limit the scope of protection defined by the claims.

This invention relates to novel small-molecule Kv7.2/3 activators and their associated pharmaceutical compositions. The pharmaceutical compositions comprise a therapeutically effective amount of the described compounds along with pharmaceutically acceptable excipients. These compositions are intended for the treatment of diseases or conditions such as epilepsy, amyotrophic lateral sclerosis (ALS), various types of pain, migraine, depression, bipolar disorder, and any neurodegenerative diseases.

### DESCRIPTION OF THE DRAWINGS

The drawings are provided to enhance understanding of the technical aspects of this application and constitute a part of the specification. They are used in conjunction with the embodiments described in this application to illustrate the technical features and should not be considered as limiting the scope of the technical aspects of this application.

FIG. 1 The Racine IV, VI seizures of the compounds of the present invention in the PTZ-induced epileptic model using ICR mice. The experimental results indicate that in the mouse PTZ epilepsy model, oral administration of the invented compound at 10 mg/kg exhibits significant activity, which is superior to the control compound.

FIG. 2 The maximum possible analgesic effect of the compounds of the present invention in the formalin model using SD rats. The experimental results indicate that in the rat formalin model, oral administration of the invented compound at 10 mg/kg exhibits significant analgesic activity, which is superior to the control compound.

FIG. 3 The structures of reference compound 1, 2, 3.

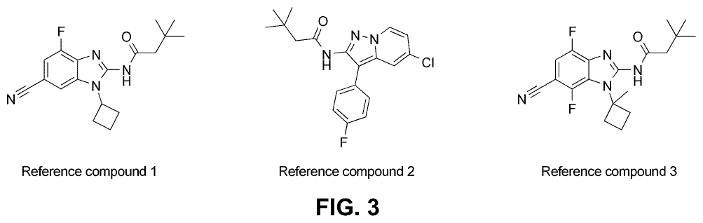

### DETAILED DESCRIPTION

Some embodiments include a compound represented by Formula (I), or its stereoisomer, tautomer, pharmaceutically acceptable salt, solvate, deuterated derivative, metabolite, or prodrug;

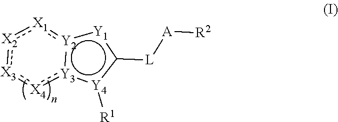

Wherein,
n is 0-5;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), halogen, C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
L is optionally selected from —NHCO— or —NHSO₂—;
A is C1-C8 alkyl, wherein A is optionally substituted with one or more substituents independently selected from fluorine (—F), chlorine (—Cl), bromine (—Br);
X₁, X₂, X₃, and X₄ are each independently selected from the group consisting of —CR³—, —CR⁴R⁵—, —N—, —NR⁶—, —O—, or —S—;
wherein

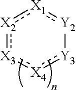

is selected from the group consisting of the following structures,

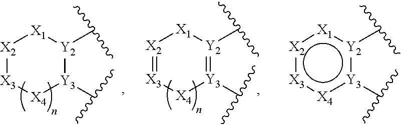

In an embodiment, each of R³, R⁴ and R⁵ is independently selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from the group consisting of —NH₂ or C1-C3 alkyl,
R⁶ is selected from the group consisting of hydrogen (H), —(C═O)(C1-C6 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;

Any one or two Xs, together with the carbon atoms to which they are attached, form a ring optionally containing 1-3 heteroatoms selected independently from nitrogen (N), oxygen (O), and sulfur (S), including

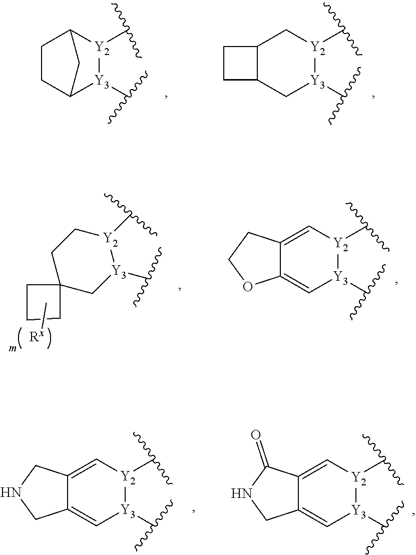

Wherein,
m is 0-6;
R^{x} is selected from the group consisting of hydrogen (H), halogen, cyano (—CN), C1-C6 alkyl;
Y₁, Y₂, Y₃, and Y₄ are each independently selected from the group consisting of C, N, O, or S;
More preferably, Y₁, Y₂, Y₃, and Y₄ are each independently selected from C or N, with examples including:
When Y₁ and Y₄ are N, Y₂ and Y₃ are C;
When Y₁ and Y₃ are N, Y₂ and Y₄ are C;
When Y₁ and Y₂ are N, Y₃ and Y₄ are C;
When Y₁, Y₂, and Y₃ are C, Y₄ is N.
Wherein,

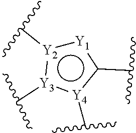

is selected from the group consisting of the following structure:

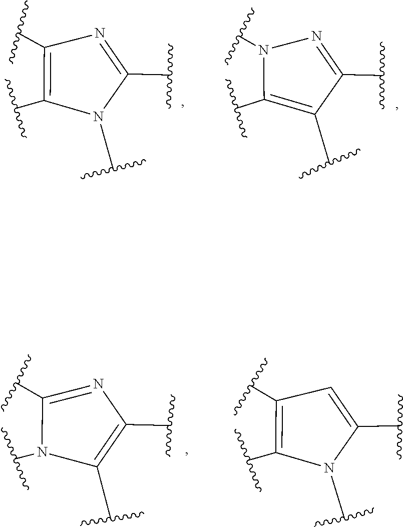

Wherein, when

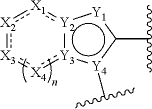

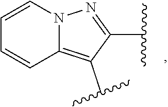 [SMILES: *c1nn2ccccc2c1*]

### L is —NHSO₂—

Wherein, when

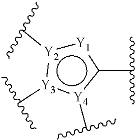

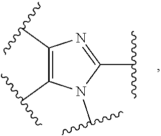 [SMILES: *c1nc(*)n(*)c1*]

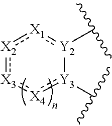

is selected from the group consisting of the following structure:

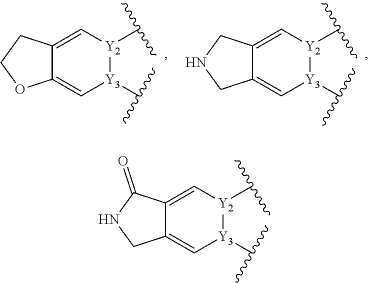

In another more specific embodiment, this invention provides or contemplates a compound of Formula (I-1), or its stereoisomers, tautomers, pharmaceutically acceptable salts, solvates, deuterated derivatives, metabolites, or prodrugs;

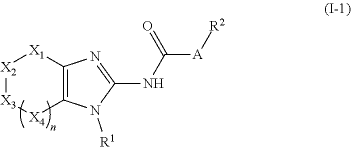

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

In some preferred embodiments, R² is selected from the group consisting of C3-C8 heterocycle or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

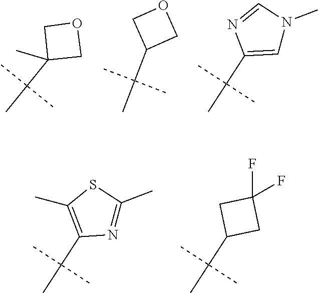

A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;

In a preferred embodiment, X₁, X₂, X₃, and X₄ are each independently selected from —CH₂—;

In an embodiment, R⁴ and R⁵ are each independently selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R¹, R⁸, and R⁹ are each independently selected from NH₂, methyl, ethyl, propyl, or isopropyl;
R⁶ is selected from the group consisting of H, —(C═O)(C1-C4 alkyl), optionally substituted C2-C6 alkenyl, optionally substituted C1-C6 alkyl, and optionally substituted C3-C8 cycloalkyl, wherein the optional substituents are independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;

In some embodiments, n is 0, 1, or 2;

In some embodiments, R¹ is optionally methyl, ethyl, propyl, isopropyl, butyl, tert-butyl, cyclopropyl, cyclobutyl, cyclopentyl, cyclohexyl, C1-C6 alkoxy, halogenated C1-C6 alkoxy;

In some embodiments, R² is optionally methyl, ethyl, propyl, isopropyl, butyl, tert-butyl, or the following groups;

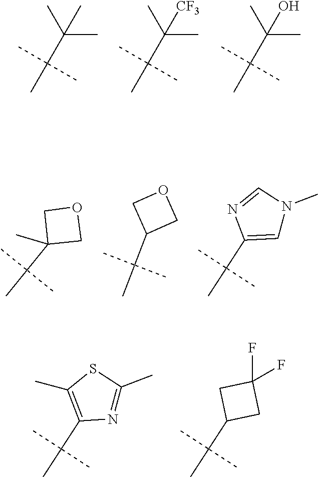

In some embodiments, A is optionally CH₂, CH₂CH₂, CH(CH₃), C(CH₃)₂, CH₂CH(CH₃), CH₂CH₂CH₂, CH₂CH₂CH₂CH₂;

In some embodiments, R³ is optionally fluorine, chlorine, bromine, iodine, CH₂F, CF₂H, CF₃, or two adjacent R³ groups and the atoms to which they are bonded together form a C3-C8 heterocycloalkyl or C3-C8 heterocycloalkenyl ring;

Preferably, n is 0 or 1;

Preferably, R¹ is optionally methyl, ethyl, tert-butyl, cyclopropyl, cyclobutyl, cyclohexyl, C1-C6 alkoxy, halogenated C1-C6 alkoxy;

Preferably, R² is optionally methyl or the following groups;

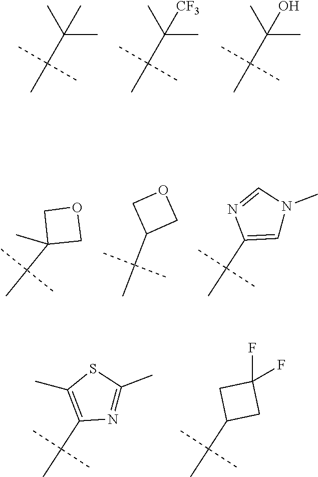

Preferably, A is selected from CH₂, CH₂CH₂, CH(CH₃), C(CH₃)₂, CH₂CH(CH₃), CH₂CH₂CH₂;

Preferably, n is 1;

Preferably, R¹ is optionally methyl, ethyl, or tert-butyl or cyclobutyl;

Preferably, R² is optionally the following groups;

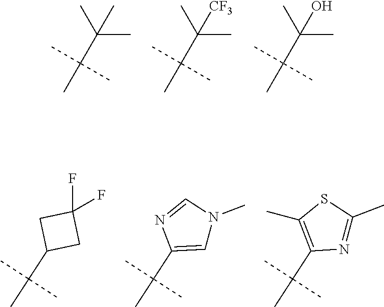

Preferably, A is CH₂;

In another more specific embodiment, this invention provides or contemplates a compound of Formula (I-2), or its stereoisomers, tautomers, pharmaceutically acceptable salts, solvates, deuterated derivatives, metabolites, or prodrugs;

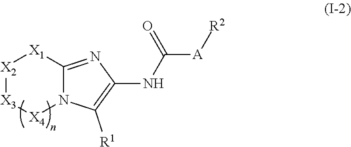

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally containing 1-4 ring heteroatoms selected independently from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;

In an embodiment, R⁴ and R⁵ are each independently selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of H, —(C═O)(C1-C4 alkyl), optionally substituted C2-C6 alkenyl, optionally substituted C1-C6 alkyl, and optionally substituted C3-C8 cycloalkyl, wherein the optional substituents are independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;

In a preferred embodiment, X₁, X₂, X₃, and X₄ are each independently selected from —CH₂—.

In some embodiments, n is 0, 1, or 2;

In some embodiments, R¹ is selected from the group consisting of methyl, ethyl, propyl, isopropyl, butyl, tert-butyl, cyclopropyl, cyclobutyl, cyclopentyl, cyclohexyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

In some embodiments, R² is selected from the group consisting of C1-C6 alkyl, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;

In some embodiments, X₁, X₂, X₃, and X₅ are each independently selected from CH₂;

Preferably, n is 0 or 1.

Preferably, R¹ is selected from the group consisting of cyclopropyl, cyclobutyl, cyclopentyl, cyclohexyl, or Ar, wherein each occurrence of R¹ is optionally substituted with one or more substituents independently selected from the group consisting of fluorine (—F), chlorine (—Cl), bromine (—Br), iodine (—I), methyl, ethyl, propyl, isopropyl, —CH₂F, —CHF₂, —CF₃, and —OCF₃; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S);

Preferably, R² is selected from the group consisting of methyl, ethyl, propyl, isopropyl, butyl, tert-butyl, or C1-C6 alkyl, wherein each occurrence of R² is optionally substituted with one or more substituents independently selected from the group consisting of OH, F, C, Br, I, CH₂F, CHF₂, or CF₃;

More preferably, n is 1;

More preferably, R¹ is selected from the group consisting of cyclohexyl or para-trifluoromethoxyphenyl;

More preferably, R² is tert-butyl.

In another more specific embodiment, this invention provides or contemplates a compound of Formula (I-3), or its stereoisomers, tautomers, pharmaceutically acceptable salts, solvates, deuterated derivatives, metabolites, or prodrugs.

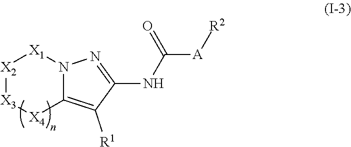

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;

In an embodiment, R⁴ and R⁵ are each independently selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of hydrogen (H), —(C═O)(C1-C4 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;

Any one or two Xs, together with the carbon atoms to which they are attached, form a C3-C6 ring optionally comprising 1 to 3 heteroatoms selected independently from nitrogen (N), oxygen (O), and sulfur (S), including but not limited to the following structure:

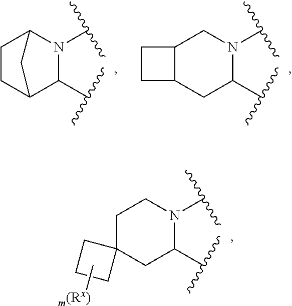

Wherein,
m is 0-6;
R^{x} is selected from the group consisting of hydrogen (H), halogen, cyano (—CN), C1-C6 alkyl;

Preferably, R⁴ and R⁵, together with the carbon atoms to which they are attached, may form a ring of the following formula,

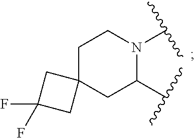 [SMILES: *C1CC2(CCN1*)CC(F)(F)C2]

or
any two Xs can be linked to form the following structure,

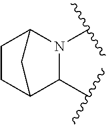 [SMILES: *C1C2CCC(C2)N1*]

In some embodiments, n is 0, 1, or 2.

In some embodiments, R¹ is selected from the group consisting of C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

In some embodiments, R² is selected from the group consisting of C1-C6 alkyl, C3-C8 heterocycle, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), methyl, ethyl, propyl, isopropyl, CH₂F, CHF₂, or CF₃;

In some embodiments, A is selected from the group consisting of CH₂, CH₂CH₂, CH(CH₃), C(CH₃)₂, CH₂CH(CH₃), CH₂CH₂CH₂, CH₂CH₂CH₂CH₂, CH(F), CH(Cl), CH(Br), or CH₂C(OH)(CH₃);

Preferably, R¹ is selected from the group consisting of cyclopropyl, cyclobutyl, cyclopentyl, cyclohexyl, C3-C8 cycloalkyl, C3-C8 cycloalkenyl, C3-C8 heterocycle, and Ar, wherein each occurrence of R¹ is optionally substituted with one or more substituents independently selected from the group consisting of CN, F, methyl; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S);

Preferably, R² is selected from the group consisting of methyl, ethyl, propyl, isopropyl, butyl, tert-butyl, C1-C6 alkyl, or C3-C8 heterocycle, wherein each occurrence of R² is optionally substituted with one or more substituents independently selected from the group consisting of F, methyl, or CF₃;

Preferably, A is selected from the group consisting of CH₂, CH₂CH₂, CH(CH₃), C(CH₃)₂, CH₂CH(CH₃), CH₂CH₂CH₂, CH₂CH₂CH₂CH₂, CH(F), CH(Cl), or CH(Br);

Preferably, R¹ is selected from the group consisting of cyclobutyl, cyclohexyl, para-fluorophenyl, para-cyanophenyl, 4-fluoro-2-methylphenyl, 3-methylisoxazole, cyclohex-1-enyl, pyridyl, or para-fluoropyridyl;

Preferably, R² is selected from the group consisting of:

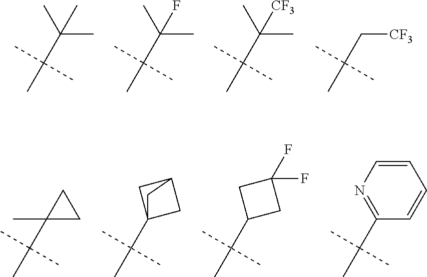

Preferably, A is selected from CH₂;

In another more specific embodiment, this invention provides or contemplates a compound of Formula (I-4) as shown below, where X₃ is optionally —CR⁴R⁵—, —NR⁶—;

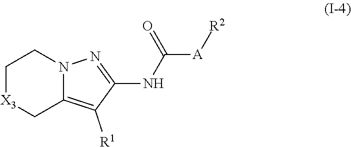

Furthermore, some embodiments may include a compound represented below,

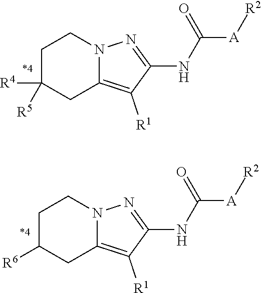

In an embodiment, R⁴ and R⁵ are each independently selected from the group consisting of hydrogen (H), halogen, hydroxyl (—OH), amino (—NH₂), cyano (—CN), —C(═O)OH, NHCOR′, —(C═O)R′, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; wherein R⁴ and R⁵ together may form a carbonyl (—C═O) functional group; R′ and R″ are each independently selected from the group consisting of C1-C6 alkyl or halogenated C1-C6 alkyl;

Preferably, R⁴ and R⁵ are each independently selected from the group consisting of hydrogen (H), halogen, hydroxyl (—OH), amino (—NH₂), cyano (—CN), —C(═O)OH, C1-C6 alkyl, or C1-C6 alkoxy, each of which is optionally substituted with one or more substituents independently selected from the group consisting of fluorine (—F), chlorine (—Cl), bromine (—Br), iodine (—I); wherein R⁴ and R⁵, together with the carbon atoms to which they are attached, form a C3-C6 ring optionally containing 1-3 heteroatoms selected independently from nitrogen (N), oxygen (O), and sulfur (S);

Preferably, R⁶ is selected from the group consisting of (C═O)methyl, —(C═O)ethyl, —(C═O)propyl, vinyl, propenyl, allyl, methyl, ethyl, propyl, isopropyl, butyl, tert-butyl, cyclopropyl, cyclobutyl, cyclopentyl, cyclohexyl, C2-C6 alkenyl, C1-C6 alkyl, or C3-C8 cycloalkyl, wherein each occurrence of R⁶ is optionally substituted with one or more substituents independently selected from the group consisting of halogen, methyl, ethyl, propyl, isopropyl;

Preferably, R⁴ is H, R⁵ is selected from H, F, Cl, Br, I, OH, CN, —C(═O)OH, CF₃, methoxy; or R⁴ and R⁵, together with the carbon atoms to which they are attached, form a C3-C6 ring optionally containing 1-3 heteroatoms selected independently from nitrogen (N), oxygen (O), and sulfur (S);

Preferably, R⁶ is optionally CH₂CH═CH₂, methyl, —(C═O)methyl, cyclopropyl, cyclobutyl, methylcyclobutane, or

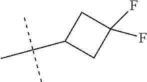

Preferably, R⁴ and R⁵, together with the carbon atoms to which they are attached, form a ring of formula,

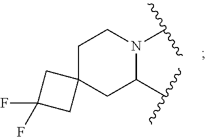 [SMILES: *C1CC2(CCN1*)CC(F)(F)C2]

Alternatively, any one or two Xs, together with the carbon atoms to which they are attached, form the following structure,

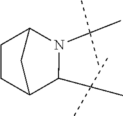

In another more specific embodiment, this invention provides or contemplates a compound of Formula (I-5), or its stereoisomers, tautomers, pharmaceutically acceptable salts, solvates, deuterated derivatives, metabolites, or prodrugs;

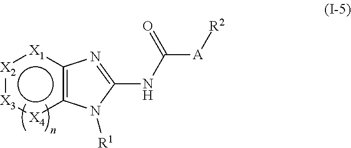

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;

Preferably, R¹ is C(CH₃)₃, R² is C(CH₃)₂CF₃, and A is CH₂,
X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;

Two adjacent R³ groups and the atoms to which they are attached together form C3-C8 heterocycloalkyl or C3-C8 heterocycloalkenyl, including but not limited to the following groups:

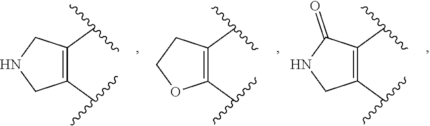 [SMILES: *C1=C(*)CNC1 ; *C1=C(*)OCC1 ; *C1=C(*)C(=O)NC1]

In another more specific embodiment, this invention provides or contemplates a compound of Formula (I-6), or its stereoisomer, tautomer, pharmaceutically acceptable salt, solvate, deuterated compound, metabolite, or prodrug:

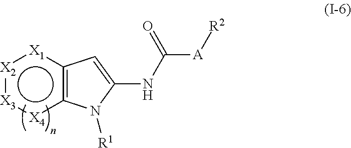

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, 9 R⁴ and R⁵ together may form a carbonyl (—C═O) functional group; R′ and R″ are each independently selected from the group consisting of C1-C6 alkyl or halogenated C1-C6 alkyl;
R¹, R⁸, and R⁹ are each independently selected from NH₂, methyl, ethyl, propyl, or isopropyl;

In some embodiments, n is 0, 1, or 2;

Preferably, n is 0 or 1;

Specifically, n is 1;

In some embodiments, R¹ is selected from the group consisting of methyl, ethyl, propyl, isopropyl, butyl, tert-butyl, cyclopropyl, cyclobutyl, cyclopentyl, cyclohexyl, C1-C6 alkyl, C3-C8 cycloalkyl, or Ar, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), methyl, ethyl, propyl, isopropyl, CH₂F, CHF₂, CF₃, OCF₃, cyclopropyl, cyclobutyl, cyclopentyl, cyclohexyl, C1-C6 alkoxy, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

Preferably, R¹ is selected from the group consisting of methyl, ethyl, propyl, isopropyl, butyl, tert-butyl, cyclopropyl, cyclobutyl, cyclopentyl, cyclohexyl, C1-C6 alkyl, C3-C8 cycloalkyl, or Ar, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), methyl, ethyl, propyl, isopropyl, CH₂F, CHF₂, CF₃, OCF₃, cyclopropyl, cyclobutyl, cyclopentyl, cyclohexyl, C1-C6 alkoxy, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

Specifically, R¹ is para-fluorophenyl;

In some embodiments, R² is selected from the group consisting of methyl, ethyl, propyl, isopropyl, butyl, tert-butyl, C1-C6 alkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), methyl, ethyl, propyl, isopropyl, CH₂F, CHF₂, CF₃, OCF₃, cyclopropyl, cyclobutyl, cyclopentyl, cyclohexyl, C1-C6 alkoxy, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

Preferably, R² is selected from the group consisting of methyl, ethyl, propyl, isopropyl, butyl, tert-butyl, C1-C6 alkyl, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I);

Specifically, R² is tert-butyl;

In some embodiments, A is selected from the group consisting of CH₂, CH₂CH₂, CH(CH₃), C(CH₃)₂, CH₂CH(CH₃), or CH₂CH₂CH₂;

Preferably, A is selected from CH₂, CH₂CH₂, CH(CH₃), C(CH₃)₂, CH₂CH(CH₃), CH₂ CH₂CH₂;

Specifically, A is CH₂;

In some preferred embodiments, X₁, X₂, X₃, and X₄ are each independently selected from —CH— or —N— to form the group consisting of the following structures:

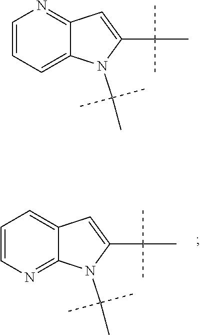

In another more specific embodiment, this invention provides or contemplates a compound of Formula (I-7), or its stereoisomer, tautomer, pharmaceutically acceptable salt, solvate, deuterated compound, metabolite, or prodrug:

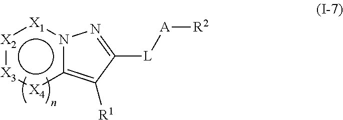

Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
L is —NHSO₂—;
A is C1-C8 alkyl, wherein A is optionally substituted with one or more substituents independently selected from fluorine (—F), chlorine (—Cl), bromine (—Br);
X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR′, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R¹, R⁸, and R⁹ are each independently selected from NH₂, methyl, ethyl, propyl, or isopropyl;

In some embodiments, n is 0, 1, or 2;

In some embodiments, R¹ is selected from the group consisting of cyclobutyl, cyclopentyl, cyclohexyl, or Ar, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), methyl, ethyl, propyl, isopropyl, CH₂F, CHF₂, CF₃, OCF₃, cyclopropyl, cyclobutyl, cyclopentyl, cyclohexyl, C1-C6 alkoxy, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

In some embodiments, R² is selected from the group consisting of methyl, ethyl, propyl, isopropyl, butyl, tert-butyl, C1-C6 alkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), methyl, ethyl, propyl, isopropyl, CH₂F, CHF₂, CF₃, OCF₃, cyclopropyl, cyclobutyl, cyclopentyl, cyclohexyl, C1-C6 alkoxy, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;

In some embodiments, A is selected from the group consisting of CH₂, CH₂CH₂, CH(CH₃), C(CH₃)₂, CH₂CH(CH₃), CH₂ CH₂CH₂, CH₂CH₂CH₂CH₂, CH(F), CH(Cl), or CH(Br);

Preferably, n is 0 or 1.

Preferably, R¹ is selected from the group consisting of cyclopropyl, cyclobutyl, cyclopentyl, cyclohexyl, or Ar, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I);

Preferably, R² is selected from the group consisting of methyl, ethyl, propyl, isopropyl, butyl, tert-butyl, C1-C6 alkyl, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), methyl, ethyl, propyl, isopropyl, CH₂F, CHF₂, or CF₃;

Preferably, A is selected from the group consisting of CH₂, CH₂CH₂, CH(CH₃), C(CH₃)₂, CH₂CH(CH₃), CH₂CH₂CH₂, CH(F), CH(Cl), or CH(Br).

More preferably, n is 1;

More preferably, R¹ is selected from cyclobutyl, cyclohexyl, or para-fluorophenyl;

More preferably, R² is selected from ethyl, isopropyl, tert-butyl, hydroxyl-substituted tert-butyl, or phenyl;

More preferably, A is CH₂ or CH(Cl);

Some embodiments are further illustrated by the compounds below, or their stereoisomer, tautomer, pharmaceutically acceptable salt, solvate, deuterated compound, metabolite, or prodrug thereof:

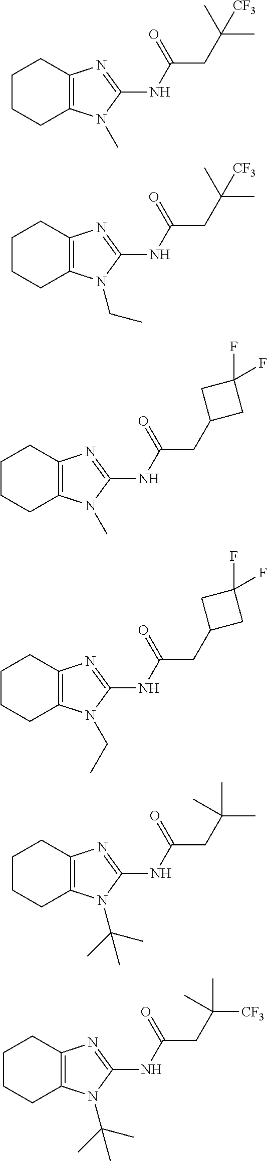 [SMILES: Cn1c(NC(=O)CC(C)(C)C(F)(F)F)nc2c1CCCC2 ; CCn1c(NC(=O)CC(C)(C)C(F)(F)F)nc2c1CCCC2 ; Cn1c(NC(=O)CC2CC(F)(F)C2)nc2c1CCCC2 ; CCn1c(NC(=O)CC2CC(F)(F)C2)nc2c1CCCC2 ; CC(C)(C)CC(=O)Nc1nc2c(n1C(C)(C)C)CCCC2 ; CC(C)(C)n1c(NC(=O)CC(C)(C)C(F)(F)F)nc2c1CCCC2]

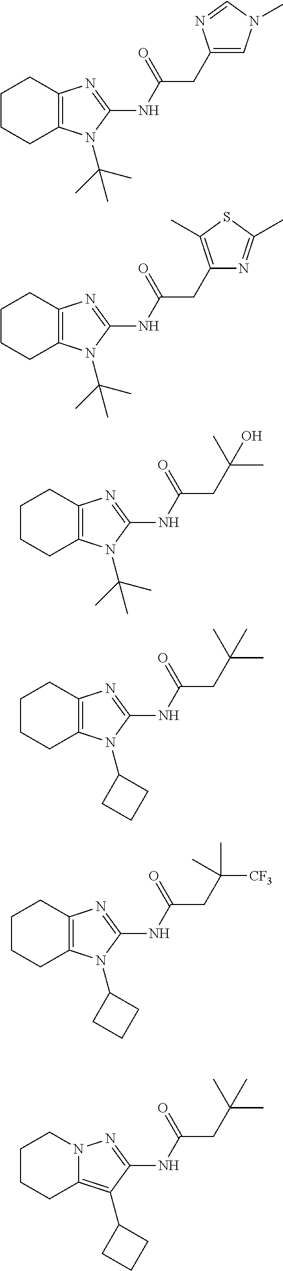 [SMILES: Cn1cnc(CC(=O)Nc2nc3c(n2C(C)(C)C)CCCC3)c1 ; Cc1nc(CC(=O)Nc2nc3c(n2C(C)(C)C)CCCC3)c(C)s1 ; CC(C)(O)CC(=O)Nc1nc2c(n1C(C)(C)C)CCCC2 ; CC(C)(C)CC(=O)Nc1nc2c(n1C1CCC1)CCCC2 ; CC(C)(CC(=O)Nc1nc2c(n1C1CCC1)CCCC2)C(F)(F)F ; CC(C)(C)CC(=O)Nc1nn2c(c1C1CCC1)CCCC2]

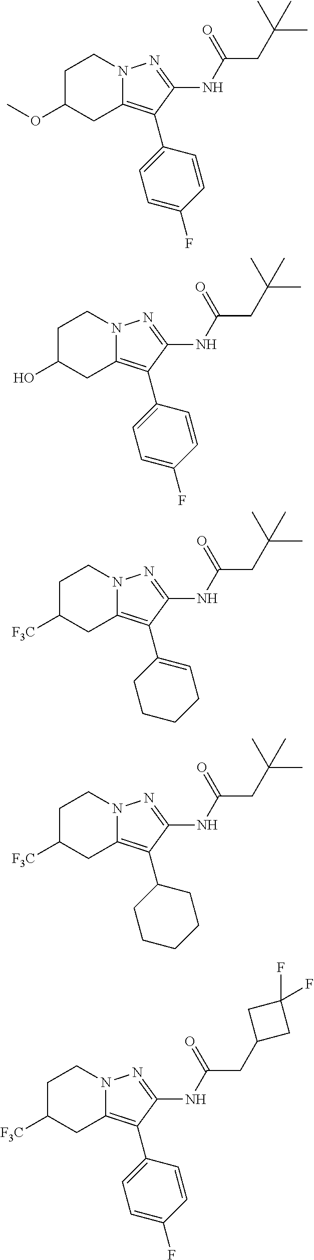

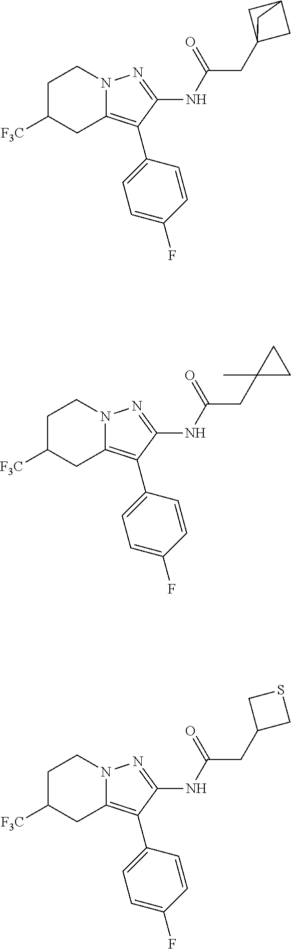 [SMILES: O=C(CC12CC(C1)C2)Nc1nn2c(c1-c1ccc(F)cc1)CC(C(F)(F)F)CC2 ; CC1(CC(=O)Nc2nn3c(c2-c2ccc(F)cc2)CC(C(F)(F)F)CC3)CC1 ; O=C(CC1CSC1)Nc1nn2c(c1-c1ccc(F)cc1)CC(C(F)(F)F)CC2]

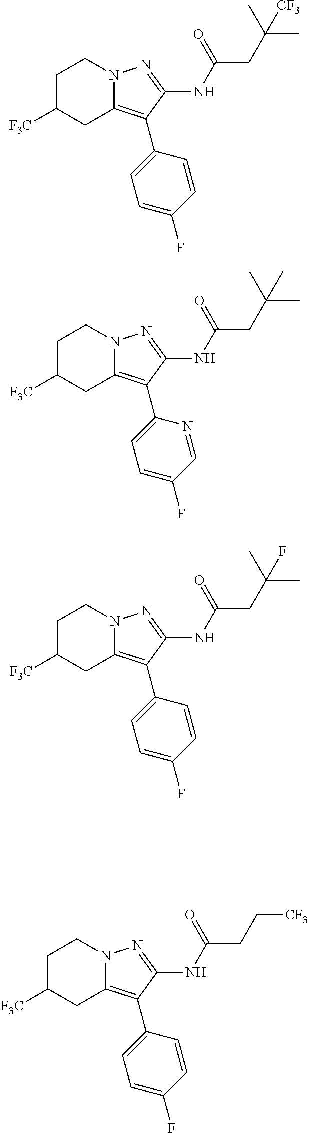 [SMILES: CC(C)(CC(=O)Nc1nn2c(c1-c1ccc(F)cc1)CC(C(F)(F)F)CC2)C(F)(F)F ; CC(C)(C)CC(=O)Nc1nn2c(c1-c1ccc(F)cn1)CC(C(F)(F)F)CC2 ; CC(C)(F)CC(=O)Nc1nn2c(c1-c1ccc(F)cc1)CC(C(F)(F)F)CC2 ; O=C(CCC(F)(F)F)Nc1nn2c(c1-c1ccc(F)cc1)CC(C(F)(F)F)CC2]

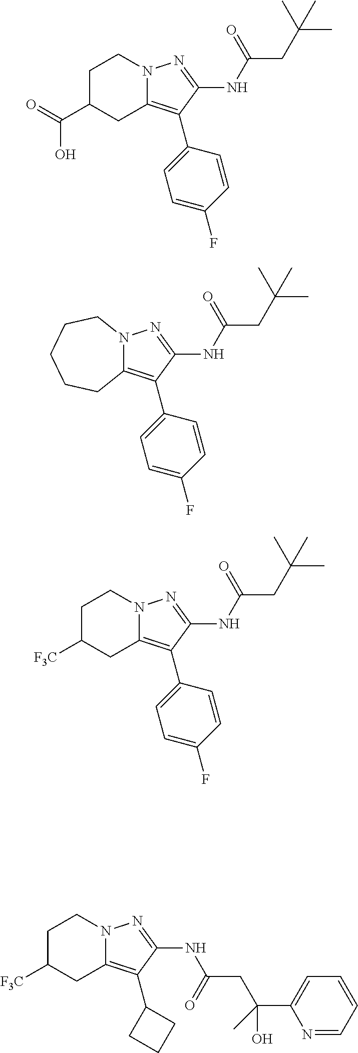 [SMILES: CC(C)(C)CC(=O)Nc1nn2c(c1-c1ccc(F)cc1)CC(C(=O)O)CC2 ; CC(C)(C)CC(=O)Nc1nn2c(c1-c1ccc(F)cc1)CCCCC2 ; CC(C)(C)CC(=O)Nc1nn2c(c1-c1ccc(F)cc1)CC(C(F)(F)F)CC2 ; CC(O)(CC(=O)Nc1nn2c(c1C1CCC1)CC(C(F)(F)F)CC2)c1ccccn1]

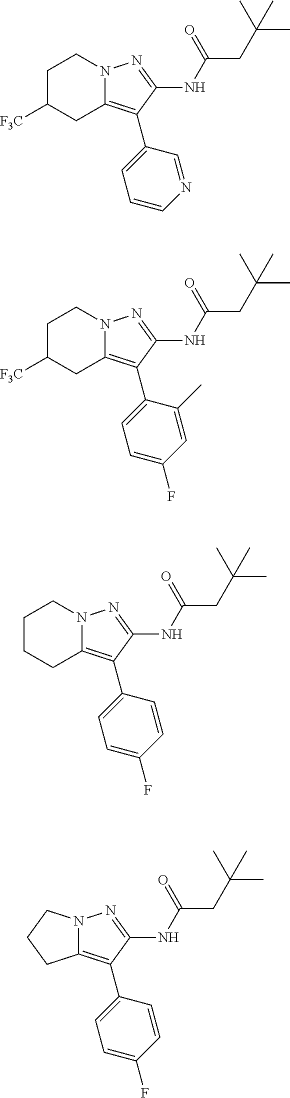 [SMILES: CC(C)(C)CC(=O)Nc1nn2c(c1-c1cccnc1)CC(C(F)(F)F)CC2 ; Cc1cc(F)ccc1-c1c(NC(=O)CC(C)(C)C)nn2c1CC(C(F)(F)F)CC2 ; CC(C)(C)CC(=O)Nc1nn2c(c1-c1ccc(F)cc1)CCCC2 ; CC(C)(C)CC(=O)Nc1nn2c(c1-c1ccc(F)cc1)CCC2]

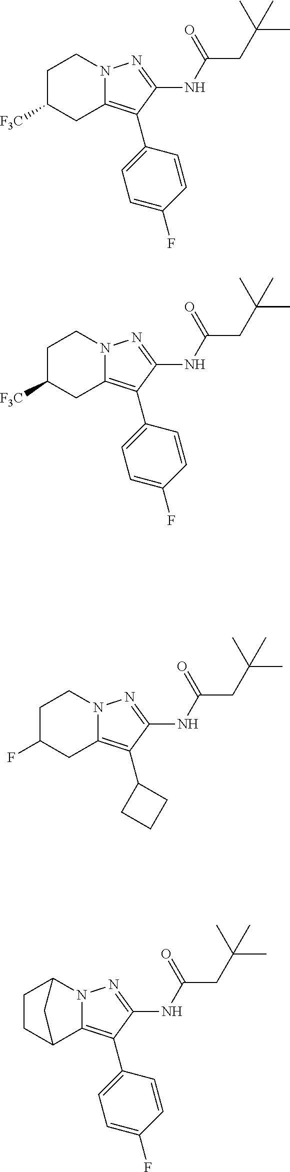 [SMILES: CC(C)(C)CC(=O)Nc1nn2c(c1-c1ccc(F)cc1)C[C@H](C(F)(F)F)CC2 ; CC(C)(C)CC(=O)Nc1nn2c(c1-c1ccc(F)cc1)C[C@@H](C(F)(F)F)CC2 ; CC(C)(C)CC(=O)Nc1nn2c(c1C1CCC1)CC(F)CC2 ; CC(C)(C)CC(=O)Nc1nn2c(c1-c1ccc(F)cc1)C1CCC2C1]

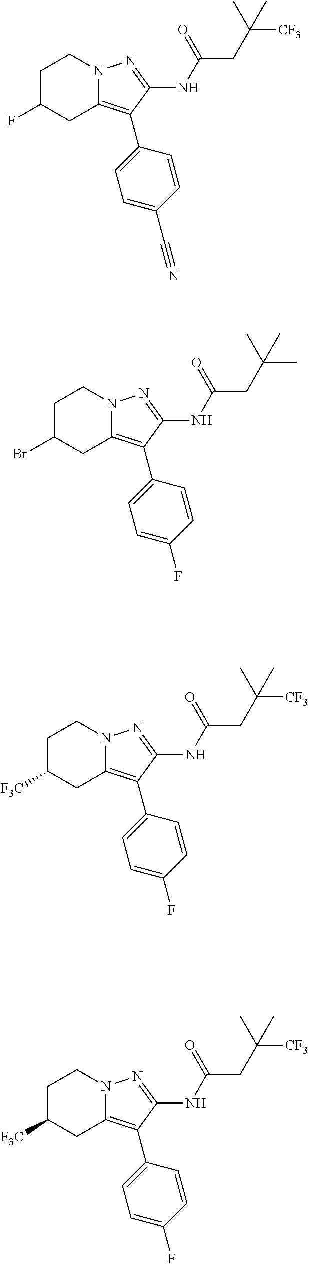 [SMILES: CC(C)(CC(=O)Nc1nn2c(c1-c1ccc(C#N)cc1)CC(F)CC2)C(F)(F)F ; CC(C)(C)CC(=O)Nc1nn2c(c1-c1ccc(F)cc1)CC(Br)CC2 ; CC(C)(CC(=O)Nc1nn2c(c1-c1ccc(F)cc1)C[C@H](C(F)(F)F)CC2)C(F)(F)F ; CC(C)(CC(=O)Nc1nn2c(c1-c1ccc(F)cc1)C[C@@H](C(F)(F)F)CC2)C(F)(F)F]

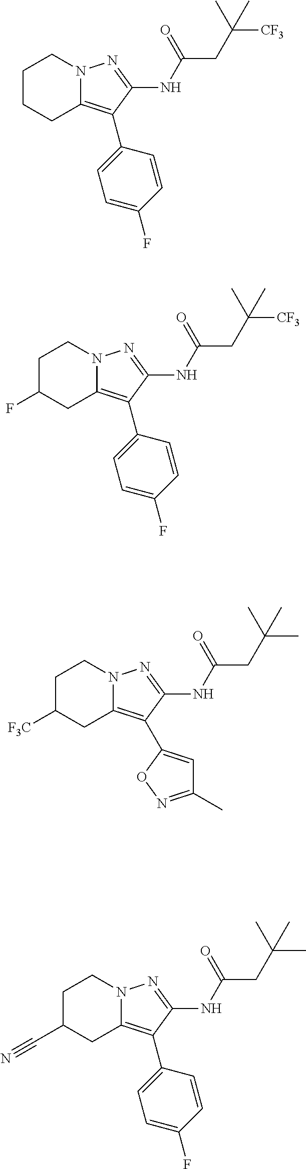 [SMILES: CC(C)(CC(=O)Nc1nn2c(c1-c1ccc(F)cc1)CCCC2)C(F)(F)F ; CC(C)(CC(=O)Nc1nn2c(c1-c1ccc(F)cc1)CC(F)CC2)C(F)(F)F ; Cc1cc(-c2c(NC(=O)CC(C)(C)C)nn3c2CC(C(F)(F)F)CC3)on1 ; CC(C)(C)CC(=O)Nc1nn2c(c1-c1ccc(F)cc1)CC(C#N)CC2]

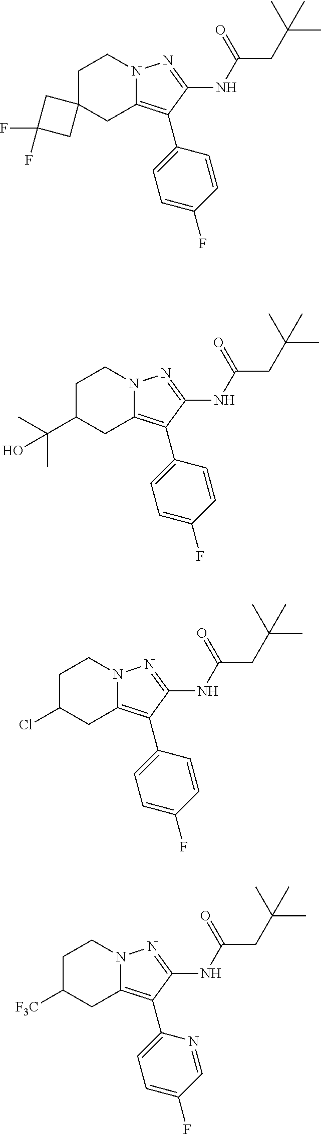

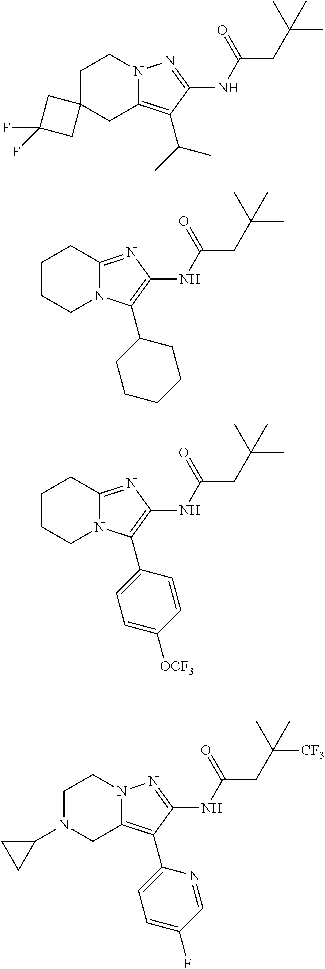

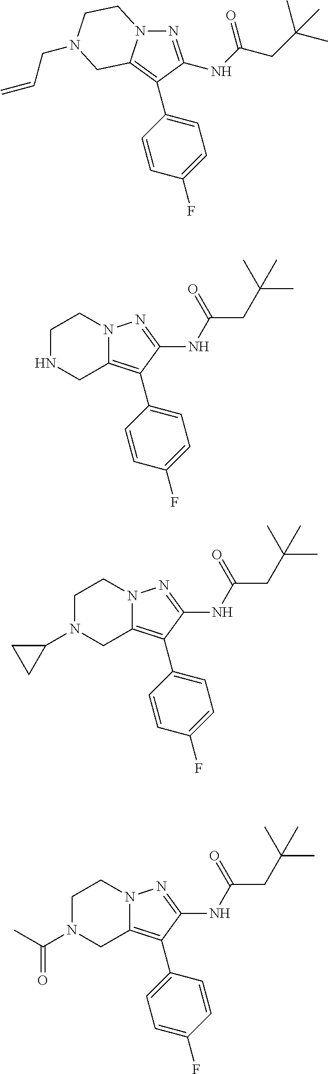

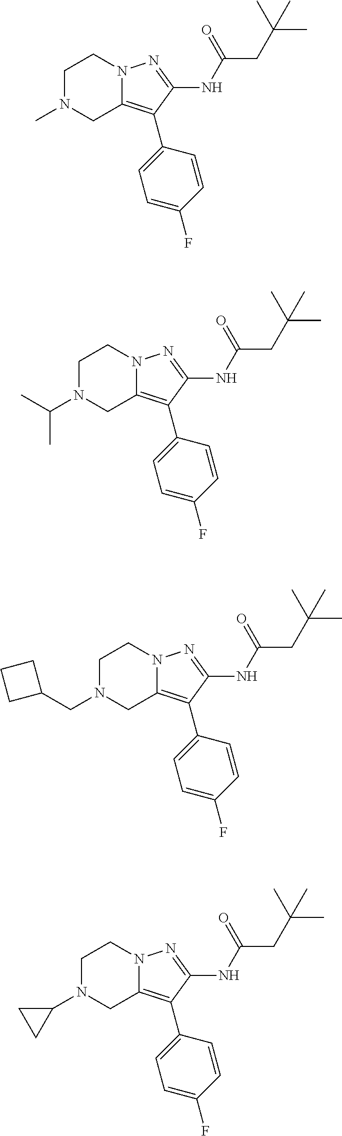

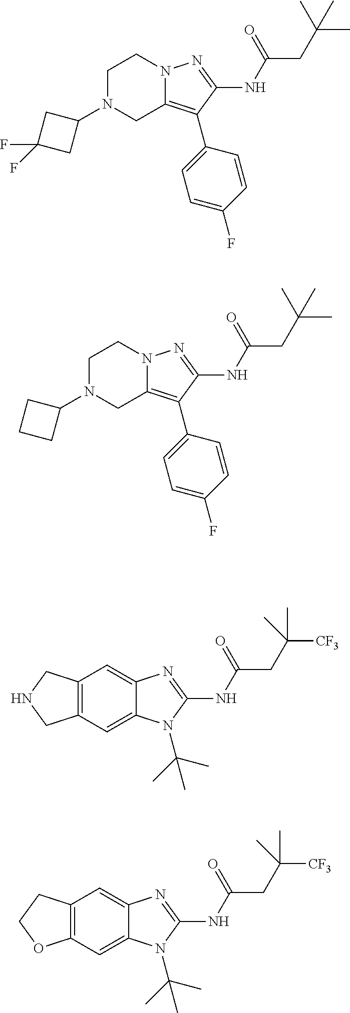 [SMILES: CC(C)(C)CC(=O)Nc1nn2c(c1-c1ccc(F)cc1)CN(C1CC(F)(F)C1)CC2 ; CC(C)(C)CC(=O)Nc1nn2c(c1-c1ccc(F)cc1)CN(C1CCC1)CC2 ; CC(C)(C)n1c(NC(=O)CC(C)(C)C(F)(F)F)nc2cc3c(cc21)CNC3 ; CC(C)(C)n1c(NC(=O)CC(C)(C)C(F)(F)F)nc2cc3c(cc21)OCC3]

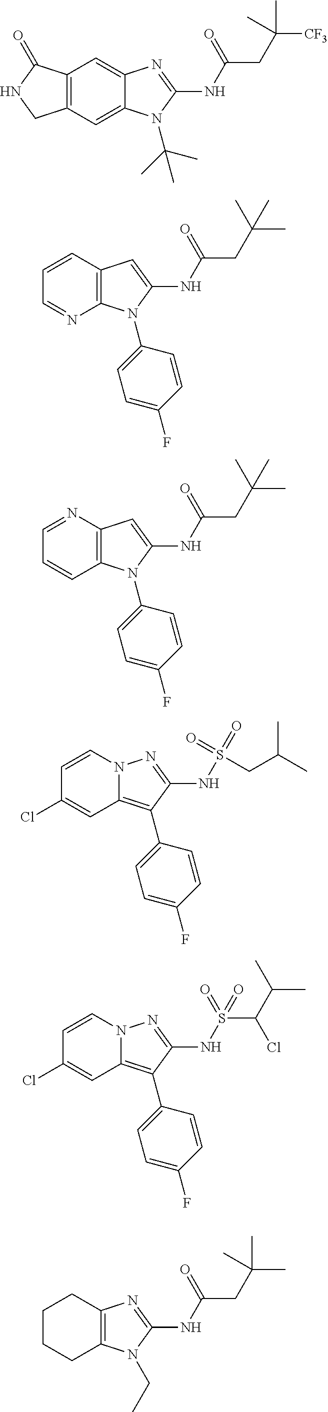

In some embodiments of the invention, one or more hydrogen atoms can be replaced by deuterium. It is well established that deuteration of physiologically active compounds offers the advantage of retaining the pharmacological profile of their hydrogen counterparts while positively impacting their metabolic outcome. Selective replacement of one or more hydrogen with deuterium, in a compound of the present invention, could improve the safety, tolerability and efficacy of the compound when compared to its all hydrogen counterpart.

Methods for incorporation of deuterium into compounds are well established. Using metabolic studies established in the art, the compound of the present invention can be tested to identify sites for selective placement of a deuterium isotope, wherein the isotope will not be metabolized. Moreover, these studies identify sites of metabolism as the location where a deuterium atom would be placed.

### Method of Use

The invention provides a pharmaceutical composition containing a compound represented by Formula (I), (I-1), (I-2), (I-3), (I-4), (I-5), (I-6) or (I-7), its stereoisomers, pharmaceutically acceptable salts, solvates, deuterated derivatives, metabolites, or prodrugs, along with at least one pharmaceutical excipient. In specific embodiments, the compound of Formula (I), (I-1), (I-2), (I-3), (I-4), (I-5), (I-6) or (I-7) is present in an effective amount in the pharmaceutical composition. In specific embodiments, the compound of Formula (I), (I-1), (I-2), (I-3), (I-4), (I-5), (I-6) or (I-7) is provided in a therapeutically effective amount. In specific embodiments, the compound of Formula (I), (I-1), (I-2), (I-3), (I-4), (I-5), (I-6) or (I-7) is provided in a prophylactically effective amount.

The invention provides the application of a compound represented by Formula (I), (I-1), (I-2), (I-3), (I-4), (I-5), (I-6) or (I-7), its stereoisomers, pharmaceutically acceptable salts, solvates, deuterated derivatives, metabolites, or prodrugs, or the pharmaceutical composition described herein, in the preparation of drugs for the prevention and/or treatment of KCNQ2/3 (Kv7.2/3) channel-related diseases.

In some embodiments, these related diseases include epilepsy, smooth muscle contractility disorder, neuropathic pain, inflammatory pain, persistent pain, cancer pain, postoperative pain, migraine, depression, anxiety, substance abuse, schizophrenia, bladder disorder, a vasculature disorder, a dyskinesia, dystonia, mania, a hearing disorder, bipolar disorder, neurodegenerative diseases or their combination.

The invention provides the application of a compound represented by Formula(I), (I-1), (I-2), (I-3), (I-4), (I-5), (I-6) or (I-7), its stereoisomers, pharmaceutically acceptable salts, solvates, deuterated derivatives, metabolites, or prodrugs, or the pharmaceutical composition described herein, in the preparation of KCNQ2/3 (Kv7.2/3) channel activators.

These drugs can be used for the treatment, alleviation, or prevention of epilepsy, smooth muscle contractility disorder, neuropathic pain, inflammatory pain, persistent pain, cancer pain, postoperative pain, migraine, depression, anxiety, substance abuse, schizophrenia, bladder disorder, a vasculature disorder, a dyskinesia, dystonia, mania, a hearing disorder, bipolar disorder, neurodegenerative diseases or their combination.

The compounds of this invention are structurally novel, and in vitro testing has demonstrated their excellent potassium channel modulation activity. Preferably, they exhibit strong agonistic activity toward the KCNQ2/3 (Kv7.2/3) channel, making them suitable for the preparation of drugs for the treatment and/or prevention of KCNQ2/3 (Kv7.2/3) channel-related diseases and conditions. Specifically, these include epilepsy, smooth muscle contractility disorder, neuropathic pain, inflammatory pain, persistent pain, cancer pain, postoperative pain, migraine, depression, anxiety, substance abuse, schizophrenia, bladder disorder, a vasculature disorder, a dyskinesia, dystonia, mania, a hearing disorder, bipolar disorder, neurodegenerative diseases or their combination.

### Terms and Definitions

Unless otherwise specified, the definitions of groups and terms described in this specification and claims, including examples, exemplary, preferred forms, descriptions in tables, and specific compounds in embodiments, can be freely combined and integrated. Thus, subsequent group definitions and compound structures should be considered within the scope described in the specification.

The compounds described herein may have asymmetric centers. Compounds of the invention containing asymmetric substituted atoms can be separated in optically active or racemic forms. Unless specifically stated otherwise regarding stereochemistry or isomeric forms, all chiral, diastereomeric, racemic, and geometrical isomers of the described structures are applicable.

As used herein, the term “halogen” means F, Cl, Br or I.

As used herein, the term “alkyl” refers to straight-chain or branched saturated aliphatic hydrocarbons containing 1 to 20 carbon atoms, preferably 1 to 8 carbon atoms, more preferably 1 to 6 carbon atoms, and further preferably 1 to 4 carbon atoms. Non-limiting examples include methyl, ethyl, n-propyl, isopropyl, n-butyl, sec-butyl, neopentyl, tert-butyl, n-pentyl, isopentyl, neopentyl, and n-hexyl and their various branched isomers. The alkyl group may optionally be substituted with 0 to 6 substituents selected from fluorine, chlorine, bromine, iodine, hydroxyl, thiol, nitro, cyano, amino, alkylamino, amide, alkenyl, alkynyl, C1-C6 alkyl, C1-C6 hydroxyalkyl, C1-C6 alkoxy, 3- to 8-membered carbocyclic, 3- to 8-membered heterocyclic, 3- to 8-membered carbocyclic-oxy, 3- to 8-membered heterocyclic-oxy, carboxyl, or carboxylate ester. For example, a “C1-C6 alkyl” group is an alkyl group having between one and six carbon atoms.

As used herein, the term “halogenated alkyl” refers to an alkyl group having the specified number of carbon atoms, wherein one or more of the hydrogen atoms of the alkyl group are replaced by halogen groups. For example, a “halogenated C1-C3 alkyl” group is an alkyl group having between one and six carbon atoms, wherein one or more of the hydrogen atoms of the alkyl group are replaced by halogen groups.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C3-C8 cycloalkyl” group could be saturated, monovalent monocyclic or bicyclic hydrocarbon rings containing 3 to 6 carbon atoms, including fused or bridged polycyclic systems such as cyclopropyl, cyclobutyl, cyclopentyl, and cyclohexyl. The C3-C6 cycloalkyl may optionally be substituted with 0 to 6 substituents selected from fluorine, chlorine, bromine, iodine, carbonyl, hydroxyl, thiol, nitro, cyano, amino, alkylamino, amide, alkenyl, alkynyl, alkyl, hydroxyalkyl, alkoxy, carbocyclic, heterocyclic, carbocyclic-oxy, heterocyclic-oxy, carboxyl, or carboxylate ester. The term “C3-C6 cycloalkyl” appearing in this document has the same definition as provided here.

As used herein, the term “alkoxy” refers to —O-alkyl. Non-limiting examples include methoxy, ethoxy, n-propoxy, isopropoxy, n-butoxy, sec-butoxy, tert-butoxy, n-pentoxy, n-hexoxy, cyclopropoxy, and cyclobutoxy. The alkyl group may optionally be substituted with 0 to 6 substituents selected from fluorine, chlorine, bromine, iodine, hydroxyl, thiol, nitro, cyano, amino, alkylamino, alkenyl, alkynyl, alkyl, hydroxyalkyl, alkoxy, carbocyclic, heterocyclic, carbocyclic-oxy, heterocyclic-oxy, carboxyl, or carboxylate ester. The term “alkoxy” appearing in this document has the same definition as provided here.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C2-C6 alkenyl” group is an alkenyl group having between two and six carbon atoms and at least one carbon-carbon double bond. Non-limiting examples include vinyl, 2-propenyl, 3-butenyl, 2-butenyl, 4-pentenyl, 3-pentenyl, 2-hexenyl, and 3-hexenyl.

As used herein, the term “cycloalkenyl” refers to a partially unsaturated cyclic hydrocarbon group containing 3 to 8 carbon atoms with at least one carbon-carbon double bond. It includes monocyclic and bicyclic systems, where bicyclic systems may include spiro, fused, or bridged rings. In some embodiments, C4-C6 cycloalkenyl is particularly preferred, referring to partially unsaturated cyclic hydrocarbon groups containing 4 to 6 carbon atoms with at least one carbon-carbon double bond. Examples of C4-C6 cycloalkenyl include but are not limited to cyclopropenyl, cyclobutenyl, cyclopentenyl, cyclopentadienyl, cyclohexenyl, and cyclohexadienyl.

As used herein, the term “aromatic” when used alone or as part of another group refers to optionally substituted homocyclic or heterocyclic conjugated planar rings or ring systems containing delocalized electrons. These aromatic groups preferably contain 5 to 14 atoms in the ring portion, including monocyclic (such as furan or benzene), bicyclic, or tricyclic groups. The term “aromatic” includes the definition of “aryl” provided below.

As used herein, the term “aryl” or “Ar” when used alone or as part of another group refers to optionally substituted homocyclic aromatic groups, preferably monocyclic or bicyclic groups containing 6 to 10 carbon atoms, such as phenyl, biphenyl, naphthyl, substituted phenyl, substituted biphenyl, or substituted naphthyl.

As used herein, the term “carbocyclic” or “carbocyclic group” refers to optionally substituted, aromatic or non-aromatic homocyclic rings or ring systems in which all atoms in the ring are carbon, preferably containing 5 or 6 carbon atoms per ring. Exemplary substituents include one or more of the following groups: hydrocarbyl, substituted hydrocarbyl, alkyl, alkoxy, acyloxy, alkenyl, alkenyloxy, aryl, aryloxy, amino, amide, aldehyde, carbamoyl, carbocyclic, cyano, ester, ether, halogen, heterocyclic, hydroxyl, ketone, ketal, phosphate, nitro, and thio.

As used herein, the term “heteroaryl” when used alone or as part of another group refers to optionally substituted aromatic groups containing at least one heteroatom in the ring, preferably monocyclic or bicyclic groups containing 5 or 6 atoms per ring. Heteroaryl groups preferably contain 1 or 2 oxygen atoms and/or 1 to 4 nitrogen atoms and are bonded to the rest of the molecule through carbon. Examples include furanyl, benzofuranyl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyrazinyl, carbazolyl, purinyl, quinolyl, isoquinolyl, imidazopyridyl, and others.

As used herein, the term “heterocycle” or “heterocyclic” refers to optionally substituted, fully saturated or unsaturated, monocyclic or bicyclic groups containing at least one heteroatom in at least one ring, preferably monocyclic or bicyclic groups containing 5 or 6 atoms per ring. Heterocyclic groups preferably contain 1 or 2 oxygen atoms and/or 1 to 4 nitrogen atoms and are bonded to the rest of the molecule through carbon or a heteroatom. Exemplary heterocyclic groups include the heteroaryl compounds mentioned above.

As used herein, the term “stereoisomers” refers to isomers that differ only in the spatial arrangement of atoms, including cis-trans isomers, enantiomers, and conformational isomers.

As used herein, the term “optionally substituted” refers to a group that is either unsubstituted or substituted with the subsequently identified substituents. For example, a group that is “optionally substituted with 1-2 halogen” is either unsubstituted, substituted with 1 halogen group, or substituted with 2 halogen groups.

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

The term “therapeutically effective amount” refers to an amount sufficient to effectively treat a disease. The effective amount varies based on compound type, disease type, severity, and patient age, but can be adjusted by skilled practitioners accordingly.

The term “pharmaceutically acceptable” refers to compounds, materials, compositions, and/or dosage forms that, within reasonable medical judgment, are suitable for contact with patient tissue without excessive toxicity, irritation, allergic response, or other complications, providing a favorable benefit-to-risk ratio and being effective for the intended use.

### Embodiments Section

The following section provides a detailed explanation of the general formula compounds of this invention, along with their preparation methods and applications, based on specific embodiments. The examples below serve as illustrative descriptions and explanations and should not be construed as limitations on the scope of the invention. Any technology implemented based on the content of this invention falls within its intended protection scope.

Unless otherwise specified, the raw materials and reagents used in the examples are commercially available or can be prepared using known methods.

Compounds are named according to conventional nomenclature rules in the field, while commercially available reagents are referred to by their supplier catalog names.

Analytical data were collected using various instruments. NMR were recorded on a Bruker Avance Neo 400 MHz liquid superconducting nuclear magnetic resonance spectrometer at 400 MHz and 600 MHz, using TMS (δ=0) as an internal standard, with DMSO-d6 or CDCl3 as solvents. MS were acquired using a Waters ACQUITY UPLC system with an ACQUITY UPLC BEH C8 column (50 mm×2.1 mm, 1.7 μm, 20180306-C8-08), with mobile phase A being 0.01% TFA/H₂O and mobile phase B being ACN, at a flow rate of 0.2 mL/min, a column temperature of 30° C., and a detection wavelength of UV-210 nm. HPLC was conducted using a Thermo UltiMate 3000 liquid chromatograph with a Venusil ASB C18 column (4.6×250 mm, 5 μm), where mobile phase A was an aqueous phosphoric acid solution (pH=1.5) and mobile phase B was ACN, with a flow rate of 1.0 mL/min, column temperature of 35° C., detection wavelength of UV-215 nm, and an injection volume of 2 μL. The gradient elution conditions included an initial phase of 95% A and 5% B for 10 minutes, followed by a transition to 20% A and 80% B for 5 minutes, and finally a return to 95% A and 5% B for 5 minutes, with the percentages representing the volume fraction of the mobile phase in the elution solvent.

### Abbreviations

AcOH: Acetic acid
ACN: Acetonitrile
DCM: Dichloromethane
DIEA: N,N-Diisopropylethylamine
DMF: Dimethylformamide
DPPA: Diphenylphosphoryl azide
EtOAc: Ethyl acetate
MTBE: tert-Butyl methyl ether
NaBH₃CN: Sodium cyanoborohydride
NIS: N-Iodosuccinimide
TEA: Triethylamine
TFA: Trifluoroacetic acid
THF: Tetrahydrofuran
SFC: Supercritical fluid chromatography
% MPE: Percent maximum potential effect
LCMS: Liquid chromatography-mass spectrometry
NMR: Nuclear magnetic resonance
HPLC: High-performance liquid chromatography
TMS: Tetramethylsilane
min: Minutes
mmol: Millimoles
mg: Milligrams
μg: Micrograms
g: Grams
kg: kilograms
eq: Equivalent
mm: Millimeters
μm: Micrometers
nm: Nanometers
mL: Milliliters
μL: Microliters
hr/h, h: Hours
M: Molar
μM: Micromolars
μmol: Micromoles
mV: Millivolts
ms: Milliseconds
MHz: Megahertz
TLC: Thin-layer chromatography
Rt: Retention time
Rf: Retention factor
EtOH: Ethanol
HATU: 1-[Bis(dimethylamino)methylene]-1H-1,2,3-triazolo[4,5-b]pyridinium 3-oxide hexafluorophosphate
ESI: Electrospray ionization
HCl: Hydrogen chloride
MTBE: Methyl tertiary-butyl ether
NH₄Cl: Ammonium chloride
NaHMDS: Sodium hexamethyldisilazide
Na₂SO₄: Sodium sulfate
HClO₄: Perchloric acid
K₂CO₃: Potassium carbonate
SiO₂: Silicon dioxide
H₂: Hydrogen gas
PdCl₂(PPh₃)₂: Bis(triphenylphosphine)palladium chloride
MeOH: Methanol
t-BuOK: Potassium tert-butoxide
NaHCO₃: Sodium bicarbonate
NH₂NH₂·H₂O: Hydrazine hydrate
KOH: Potassium hydroxide
t-BuONa: Sodium tert-butoxide
NH₄HCO₃: Ammonium bicarbonate
Pd(dppf)Cl₂: (1,1′-Bis(diphenylphosphino)ferrocene)palladium(II) dichloride
Pd/C: Palladium on carbon
DPPA: Diphenylphosphoryl azide
Cs₂CO₃: Caesium carbonate
t-BuOH: tert-Butyl alcohol
DMEM: Dulbecco's modified eagle medium
FBS: Fetal bovine serum
DMSO: Dimethyl sulfoxide
NaCl: Sodium chloride
KCl: Potassium chloride
MgCl₂: Magnesium chloride
CaCl₂: Calcium chloride
HEPES: 4-(2-Hydroxyethyl)piperazine-1-ethanesulfonic acid
NaH₂PO₄·2H₂O: Sodium dihydrogen phosphate dihydrate
EGTA: Ethylene glycol tetraacetic acid
Mg-ATP: Adenosine 5′-triphosphate magnesium salt
EDTA: Disodium ethylenediaminetetraacetic acid
rpm: round per minute
KCNQ: potassium channel, subfamily Q
SPF: Specific pathogen free
ICR: Institute of cancer research
SD: Sprague dawley
SnCl₂·2H₂O: Tin(II) chloride dihydrate
CbzCl: Benzyl chloroformate
N₂: Nitrogen gas
i.p.: Intraperitoneal
PTZ: Pentylenetetrazol

### EXAMPLES

### Example 1: Synthesis of Compound 1

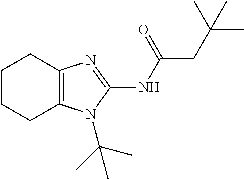 [SMILES: CC(C)(C)CC(=O)Nc1nc2c(n1C(C)(C)C)CCCC2]

### Step 1. Preparation of Intermediate 1a

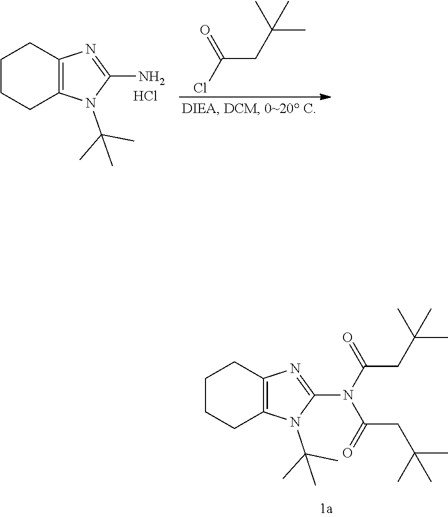

To a solution of compound 1-(tert-butyl)-4,5,6,7-tetrahydro-1H-benzo[d]imidazol-2-amine (100 mg, 435 μmol, 1.00 eq, HCl) in DCM (1.00 mL) was added DIEA (141 mg, 1.09 mmol, 190 μL, 2.50 eq) and 3,3-dimethylbutanoyl chloride (117 mg, 871 μmol, 121 μL, 2.00 eq) at 0° C. The mixture was stirred at 20° C. for 2 hrs. The reaction mixture was poured into H₂O (20.0 mL) and extracted with DCM (20.0 mL×3). The combined organic layers were washed with brine (20.0 mL), dried over Na₂SO₄, filtered and concentrated under reduced pressure to give a residue. The residue was purified by prep-TLC (SiO₂, Petroleum ether/Ethyl acetate=6/1, R_{f}=0.57) to give compound 1a (110 mg, 282 μmol, 64.9% yield) as a yellow solid.

LCMS: Rt=0.428 min, m/z: 390.6, M+H⁺

### Step 2. Preparation of Compound 1

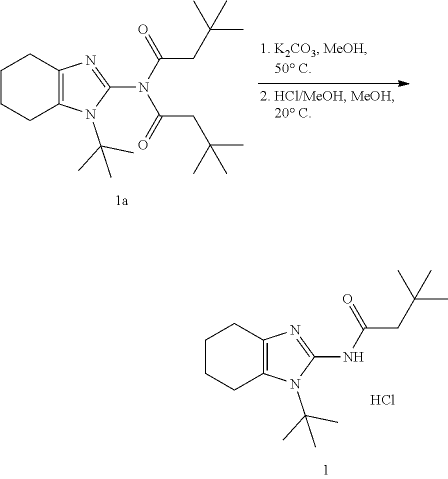 [SMILES: CC(C)(C)CC(=O)N(C(=O)CC(C)(C)C)c1nc2c(n1C(C)(C)C)CCCC2 ; CC(C)(C)CC(=O)Nc1nc2c(n1C(C)(C)C)CCCC2 ; Cl]

To a solution of compound 1a (110 mg, 282 μmol, 1.00 eq) in MeOH (2.00 mL) was added K₂CO₃ (78.1 mg, 565 μmol, 2.00 eq). The mixture was stirred at 50° C. for 2 hrs. The reaction mixture was concentrated under reduced pressure to give a residue. The residue was purified by prep-HPLC (HCl condition; column: Phenomenex luna C18 150*25 mm*10 um; mobile phase: [water (HCl)-ACN]; gradient: 16%-46% B over 10 min) to give the compound. The compound was added to HCl/MeOH (2M, 0.5 mL, 5.00 eq) and stirred at 20° C. for 2 hrs. The reaction mixture was concentrated under reduced pressure to give compound 1 (58.0 mg, 172 μmol, 60.8% yield, 97.1% purity, HCl) as an off-white solid.

LCMS: Rt=0.296 min, m/z: 292.2, M+H⁺

HPLC: Rt=1.199 min

¹H NMR: 400 MHz, DMSO-d6

δ 14.3 (s, 1H), 12.2 (s, 1H), 2.86 (s, 2H), 2.58 (s, 2H), 2.35 (s, 2H), 1.76-1.77 (m, 4H), 1.66 (s, 9H), 1.05 (s, 9H).

The preparation of the compounds listed below refers to Example 1

| Name | Structure | Analytical Data |
|---|---|---|
| Compound 2 | 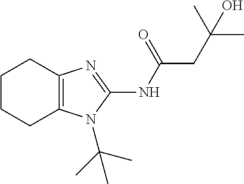 [SMILES: CC(C)(O)CC(=O)Nc1nc2c(n1C(C)(C)C)CCCC2] | ¹H NMR 400 MHz, DMSO-d₆ δ 2.91-2.94 (m, 2H), 2.61-2.64 (m, 4H), 1.84-1.91 (m, 4H), 1.76 (s, 9H), 1.37 (s, 6H). MS m/z (ESI): 294.1, M + H⁺ Purity by HPLC: 97.3% |
| Compound 3 | 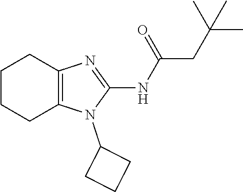 [SMILES: CC(C)(C)CC(=O)Nc1nc2c(n1C1CCC1)CCCC2] | 1H NMR (600 MHz, DMSO- d₆) δ 9.60 (s, 1H), 2.65 (s, 2H), 2.43 (s, 2H), 2.37 (s, 2H), 2.24 (d, J = 8.6 Hz, 2H), 2.15 (s, 2H), 1.77-1.64 (m, 6H), 1.23 (s, 1H), 1.02 (s, 9H). MS m/z (ESI): 290.31, M + H⁺ Purity by HPLC: 96.72% |
| Compound 4 | 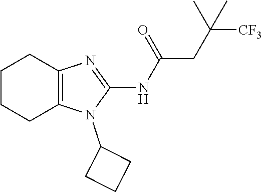 [SMILES: CC(C)(CC(=O)Nc1nc2c(n1C1CCC1)CCCC2)C(F)(F)F] | 1H NMR (600 MHz, DMSO- d₆) δ 9.90 (s, 1H), 2.63 (s, 2H), 2.44 (s, 2H), 2.37 (s, 2H), 2.26 (t, J = 9.2 Hz, 2H), 1.72 (d, J = 27.7 Hz, 7H), 1.25 (s, 8H). MS m/z (ESI): 344.26, M + H⁺ Purity by HPLC: 94.66% |
| Compound 5 | 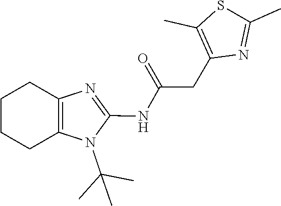 [SMILES: Cc1nc(CC(=O)Nc2nc3c(n2C(C)(C)C)CCCC3)c(C)s1] | 1H NMR (400 MHz, DMSO- d₆) δ 14.5 (s, 1H), 11.5 (s, 1H), 3.87 (s, 2H), 2.85 (s, 2H), 2.56-2.59 (m, 3H), 2.50-2.54 (m, 2H), 2.36 (s, 3H), 1.75- 1.79 (m, 4H), 1.58 (s, 9H). MS m/z (ESI): 347.2, M + H⁺ Purity by HPLC: 99.5% |
| Compound 6 | 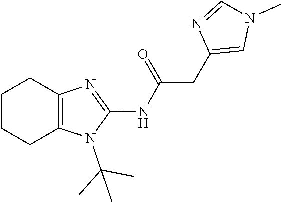 [SMILES: Cn1cnc(CC(=O)Nc2nc3c(n2C(C)(C)C)CCCC3)c1] | 1H NMR (400 MHz, DMSO- d₆) δ 14.7 (s, 1H), 12.2 (s, 1H), 9.07 (s, 1H), 7.65 (s, 1H), 4.06 (s, 2H), 3.85 (s, 3H), 2.86-2.88 (s, 2H), 2.56-2.58 (s, 2H), 1.75-1.79 (m, 4H), 1.67 (s, 9H). MS m/z (ESI): 316.2, M + H⁺ Purity by HPLC: 99.5% |
| Compound 64 | 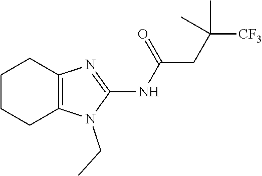 [SMILES: CCn1c(NC(=O)CC(C)(C)C(F)(F)F)nc2c1CCCC2] | 1H NMR (300 MHz, CDCl3) δ 9.03 (s, 1H), 3.84 (q, J = 7.2 Hz, 2H), 2.52 (s, 2H), 2.45 (d, J = 4.6 Hz, 4H), 1.81 (d, J = 3.8 Hz, 4H), 1.29 (s, 6H), 1.25 (t, J = 7.2 Hz, 3H). |
| Compound 65 | 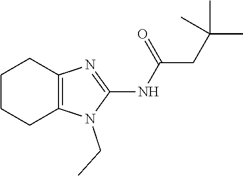 [SMILES: CCn1c(NC(=O)CC(C)(C)C)nc2c1CCCC2] | ¹H NMR (300 MHz, CDCl₃) δ 3.90 (q, J = 7.2 Hz, 2H), 2.50 (s, 4H), 2.30 (s, 2H), 1.81 (d, J = 3.6 Hz, 4H), 1.31 (t, J = 7.2 Hz, 3H), 1.06 (s, 9H). |

### Example 2: Synthesis of Compound 7

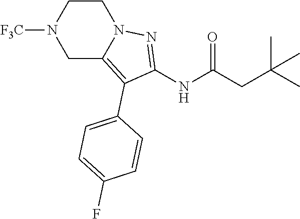 [SMILES: CC(C)(C)CC(=O)Nc1nn2c(c1-c1ccc(F)cc1)CN(C(F)(F)F)CC2]

### Step 1. Preparation of Intermediate 7a

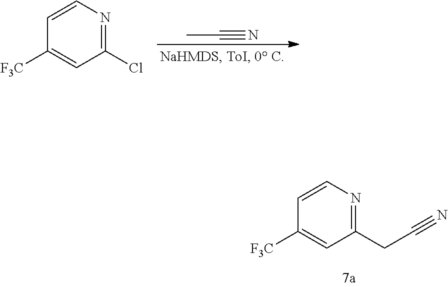

To a solution of compound 2-chloro-4-(trifluoromethyl)pyridine (4.10 g, 22.6 mmol, 1.00 eq) in Tol. (40.0 mL) was added dropwise acetonitrile (1.21 g, 29.4 mmol, 1.55 mL, 1.30 eq) at 0° C. After addition, the mixture was stirred at this temperature for 30 min, and then NaHMDS (1 M, 33.9 mL, 1.50 eq) was added dropwise at 0° C. The resulting mixture was stirred at 0° C. for 3.5 hrs. The reaction mixture was diluted with aqueous NH₄Cl (200 mL) and extracted with EtOAc (100 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=100/1 to 10/1, R_{f}=0.36) to give compound 7a (2.70 g, 14.2 mmol, 62.8% yield, 97.7% purity) as a yellow oil.

LCMS: Rt=0.267 min, m/z: 187.1 M+H⁺

¹H NMR: 400 MHz, CDCl3

δ 8.82 (d, J=5.2 Hz, 1H), 7.68 (s, 7.2H), 7.54 (d, J=4.4 Hz, 1H), 4.07 (s, 9H).

### Step 2. Preparation of Intermediate 7b

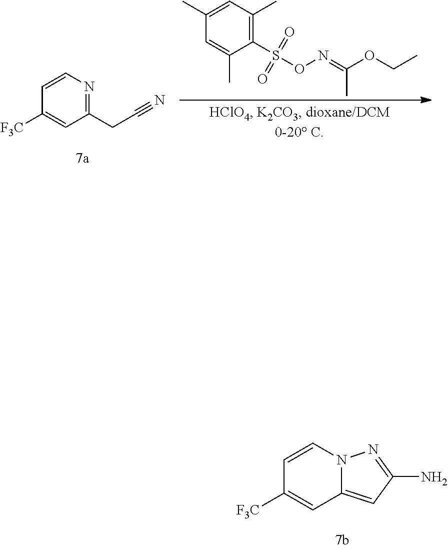 [SMILES: N#CCc1cc(C(F)(F)F)ccn1 ; CCO/C(C)=N/OS(=O)(=O)c1c(C)cc(C)cc1C ; Nc1cc2cc(C(F)(F)F)ccn2n1]

To a solution of compound ethyl (E)-N-((mesitylsulfonyl)oxy)acetimidate (6.21 g, 21.8 mmol, 1.50 eq) in dioxane (50.0 mL) was added HClO₄ (4.37 g, 43.5 mmol, 2.63 mL, 3.00 eq), and the mixture was stirred at 0° C. for 0.5 hrs. After the addition of ice-water, the precipitate was collected. The cake was dissolved in dichloromethane (20.0 mL) and dried over Na₂SO₄. To the mixture was added a solution of compound 7a (2.70 g, 14.5 mmol, 1.00 eq) in DCM (20.0 mL), and then the mixture was stirred at 20° C. for 12 hrs. To a solution of the crude material in methanol (20.0 mL) was added K₂CO₃ (500 mg) at 0° C., and then the mixture was stirred at 20° C. for 2 hrs. After the addition of water, the mixture was extracted with ethyl acetate (20.0 mL×3). The combined organic layers were washed with brine (30.0 mL), dried over Na₂SO₄, filtered and concentrated under reduced pressure to give a residue. The residue was purified by column chromatography (SiO₂, Petroleum ether/Ethyl acetate=80/1 to 10/1, R_{f}=0.27) to give compound 7b (1.35 g, 6.68 mmol, 46.1% yield, 99.6% purity) as a yellow solid.

LCMS: Rt=0.314 min, m/z: 202.0, M+H⁺

LCMS: Rt=0.318 min, m/z: 202.1, M+H⁺

### Step 3. Preparation of Intermediate 7c

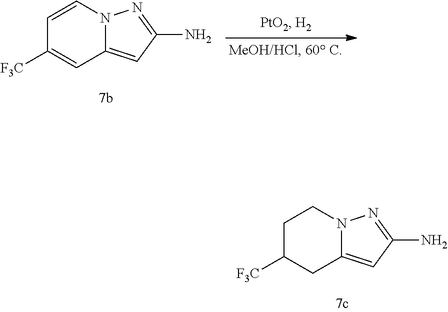 [SMILES: Nc1cc2cc(C(F)(F)F)ccn2n1 ; Nc1cc2n(n1)CCC(C(F)(F)F)C2]

To a solution of compound 7b (500 mg, 2.49 mmol, 1.00 eq) and HCl (12 M, 207 μL, 1.00 eq) in MeOH (5.00 mL) was added PtO₂ (564 mg, 2.49 mmol, 1.00 eq) under N₂ atmosphere. The suspension was degassed and purged with H₂ for 3 times. The mixture was stirred under H₂ (50 Psi) at 60° C. for 12 hrs. The reaction mixture was filtered and concentrated under reduced pressure to give a residue. The residue was purified by column chromatography (SiO₂, Petroleum ether/Ethyl acetate=50/1 to 0/1, R_{f}=0.04) to give compound 7c (334 mg, 1.63 mmol, 65.5% yield, 100% purity) as a white solid.

LCMS: Rt=0.145 min, m/z: 206.1 M+H⁺

LCMS: Rt=0.144 min, m/z: 206.1 M+H⁺

### Step 4. Preparation of Intermediate 7d

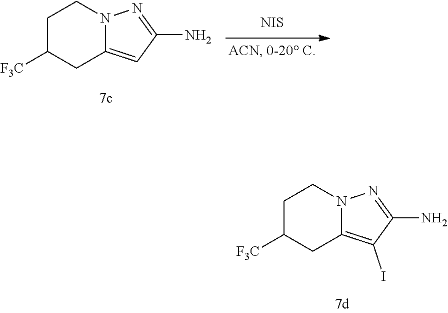 [SMILES: Nc1cc2n(n1)CCC(C(F)(F)F)C2 ; "NIS" ; Nc1nn2c(c1I)CC(C(F)(F)F)CC2]

To a solution of compound 7c (430 mg, 2.10 mmol, 1.00 eq) in ACN (5.00 mL) was added NIS (472 mg, 2.10 mmol, 1.00 eq) at 0° C. The mixture was stirred at 20° C. for 2 hrs under N₂ atmosphere. The reaction mixture was diluted with H₂O (20.0 mL) and extracted with EtOAc (10.0 mL×3). The combined organic layers were washed with brine (10.0 mL), dried over Na₂SO₄, filtered and concentrated under reduced pressure to give a residue. The residue was purified by column chromatography (SiO₂, Petroleum ether/Ethyl acetate=20/1 to 0/1, R_{f}=0.48) to give compound 7d (610 mg, 1.75 mmol, 83.7% yield, 95.2% purity) as a yellow solid.

LCMS: Rt=0.342 min, m/z: 332.0, M+H⁺

### Step 5. Preparation of Intermediate 7e

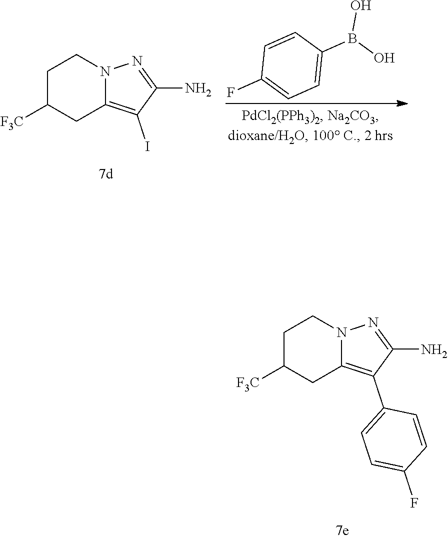

To a solution of compound 7d (610 mg, 1.84 mmol, 1.00 eq) and (4-fluorophenyl)boronic acid (258 mg, 1.84 mmol, 1.00 eq) in dioxane (3.00 mL) and H₂O (3.00 mL) was added Na₂CO₃ (391 mg, 3.68 mmol, 2.00 eq) and PdCl₂(PPh₃)₂ (129 mg, 184 μmol, 0.1 eq). The mixture was stirred at 100° C. for 2 hrs under N₂ atmosphere. The reaction mixture was diluted with H₂O (20.0 mL) and extracted with EtOAc (20.0 mL×3). The combined organic layers were washed with brine (20.0 mL), dried over Na₂SO₄, filtered and concentrated under reduced pressure to give a residue. The residue was purified by column chromatography (SiO₂, Petroleum ether/Ethyl acetate=20/1 to 0/1, R_{f}=0.18) to give compound 7e (245 mg, 819 μmol, 44.4% yield) was obtained as a yellow solid.

LCMS: Rt=0.368 min, m/z: 300.1, M+H⁺

### Step 6. Preparation of Intermediate 7f

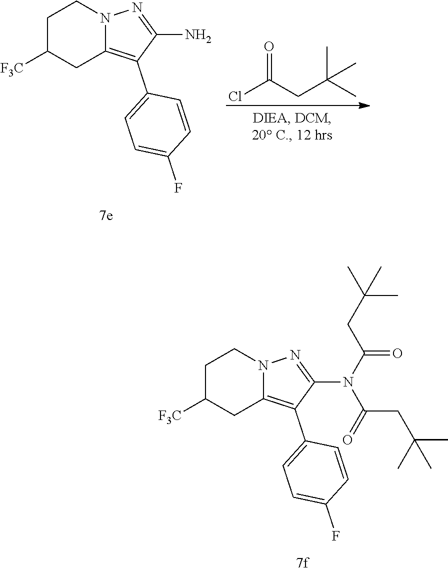

To a solution of compound 7e (245 mg, 819 μmol, 1.00 eq) in DCM (3.00 mL) was added DIEA (317 mg, 2.46 mmol, 428 μL, 3.00 eq) and 3,3-dimethylbutanoyl chloride (220 mg, 1.64 mmol, 227 μL, 2.00 eq). The mixture was stirred at 20° C. for 12 hrs. The reaction mixture was diluted with H₂O (50.0 mL) and extracted with DCM (20.0 mL×3). The combined organic layers were washed with brine (20.0 mL), dried over Na₂SO₄, filtered and concentrated under reduced pressure to give a residue to compound 7f (405 mg, crude) as a yellow oil.

LCMS: Rt=0.582 min, m/z: 496.3, M+H⁺

### Step 7. Preparation of Compound 7

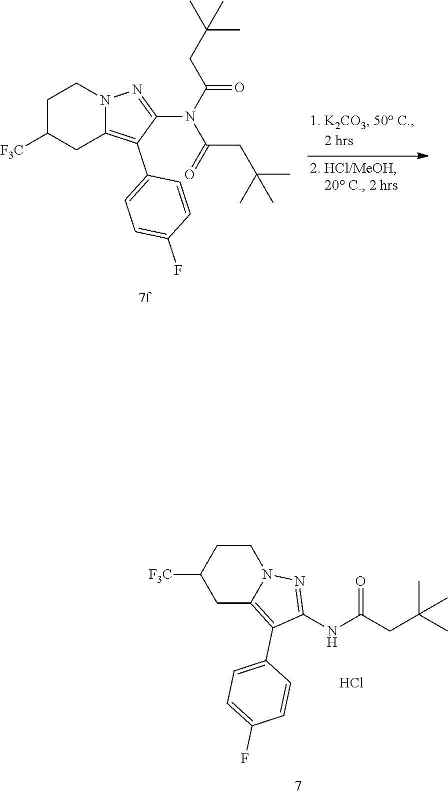 [SMILES: CC(C)(C)CC(=O)N(C(=O)CC(C)(C)C)c1nn2c(c1-c1ccc(F)cc1)CC(C(F)(F)F)CC2 ; CC(C)(C)CC(=O)Nc1nn2c(c1-c1ccc(F)cc1)CC(C(F)(F)F)CC2 ; Cl]

To a solution of compound 7f (405 mg, 817 μmol, 1.00 eq) in MeOH (5.00 mL) was added K₂CO₃ (226 mg, 1.63 mmol, 2.00 eq). The mixture was stirred at 50° C. for 2 hrs. The reaction mixture was quenched by addition H₂O (20.0 mL) and then extracted with EtOAc (20.0 mL×2). The combined organic layers were washed with brine (20.0 mL), dried over Na₂SO₄, filtered and concentrated under reduced pressure to give a residue. The residue was purified by prep-HPLC (HCl condition; column: CD04-Welch Utimate C18 150*25*7 μm; mobile phase: [water (HCl)-ACN]; gradient: 39%-69% B over 10 min) to give the compound. The compound was added to HCl/MeOH (2M, 0.5 mL, 5.00 eq) and stirred at 20° C. for 2 hrs. The reaction mixture was concentrated under reduced pressure to give compound 7 (280 mg, 705 μmol, 86.2% yield, 100% purity) as a white solid.

LCMS: Rt=0.456 min, m/z: 398.2, M+H⁺

LCMS: Rt=2.450 min, m/z: 398.2, M+H⁺

¹H NMR: 400 MHz, DMSO-d₆

¹⁹F NMR: 400 MHz, DMSO-d₆

δ 9.42 (s, 1H), 7.35-7.38 (m, 2H), 7.16-7.20 (m, 2H), 4.23-4.26 (m, 1H), 4.04-4.09 (m, 1H), 2.91-3.01 (m, 3H), 2.28 (d, J=12.4H, 1H), 2.03-2.08 (m, 3H), 0.94 (s, 9H).

The preparation of the compounds listed below refers to Example 2

| Name | Structure | Data |
|---|---|---|
| Compound 8 | 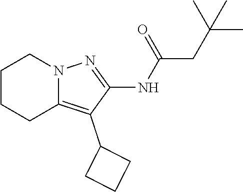 [SMILES: CC(C)(C)CC(=O)Nc1nn2c(c1C1CCC1)CCCC2] | ¹H NMR 400 MHz, CDCl3 δ 9.37 (s, 1H), 3.93 (t, J = 6.0 Hz, 2H), 3.25-3.30 (m, 1H), 2.75 (t, J = 6.4 Hz, 2H), 2.09- 2.16 (m, 6H), 1.91-1.92 (m, 2H), 1.76-1.78 (m, 4H), 1.02 (s, 9H). MS m/z (ESI): 290.2, M + H⁺ Purity by HPLC: 99.2% |
| Compound 9 | 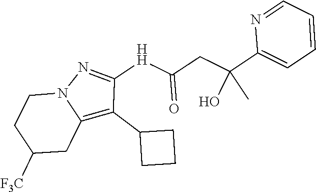 | ¹H NMR 400 MHz, DMSO-d₆ δ 9.48 (d, J = 4.4 Hz, 1H), 8.50 (d, J = 8.0 Hz, 1H), 7.77- 7.81 (m, 1H), 7.68 (d, J = 8.0 Hz, 1H), 7.23-7.26 (m, 1H), 5.93-5.99 (m, 1H), 4.03-4.10 (m, 1H), 3.92-3.93 (m, 1H), 2.96-3.06 (m, 4H), 2.72-2.76 (m, 2H), 2.29-2.45 (m, 1H), 1.46 (s, 9H). MS m/z (ESI): 423.4, M + H⁺ Purity by HPLC: 95.2% |
| Compound 10 | 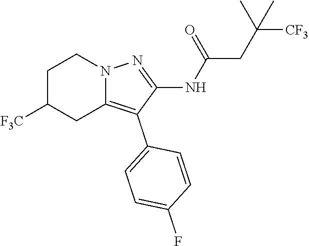 [SMILES: CC(C)(CC(=O)Nc1nn2c(c1-c1ccc(F)cc1)CC(C(F)(F)F)CC2)C(F)(F)F] | ¹H NMR 400 MHz, DMSO-d₆ δ 9.70 (s, 1H), 7.33-7.36 (m, 2H), 7.17-7.22 (m, 2H), 4.23- 4.27 (m, 1H), 4.05-4.08 (m, 1H), 2.90-32.99 (m, 3H), 2.38 (s, 2H), 2.26-2.30 (m, 1H), 2.06-2.20 (m, 1H), 1.14 (d, J = 2.4 Hz, 6H). MS m/z (ESI): 452.3, M + H⁺ Purity by HPLC: 98.7% |
| Compound 11 | 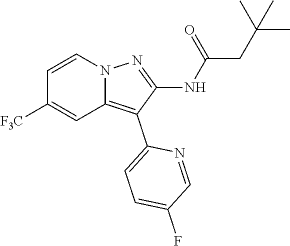 | ¹H NMR 400 MHz, DMSO-d₆ δ 9.74 (s, 1H), 8.57 (d, J = 2.8 Hz, 1H), 7.68-7.72 (m, 1H), 7.50-7.53 (m, 1H), 4.23-4.27 (m, 1H), 4.08-4.12 (m, 1H), 3.31-3.34 (m, 1H), 2.98-3.03 (m, 1H), 2.94-2.96 (m, 1H), 2.27-2.30 (m, 1H), 2.17 (s, 2H), 2.05-2.08 (m, 1H), 0.99 (s, 9H). MS m/z (ESI): 399.2, M + H⁺ Purity by HPLC: 99.0% |
| Compound 12 | 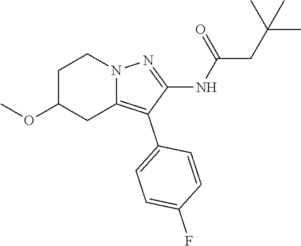 [SMILES: COC1CCn2nc(NC(=O)CC(C)(C)C)c(-c3ccc(F)cc3)c2C1] | ¹H NMR 400 MHz, DMSO-d₆ δ 9.31 (s, 1H), 7.33 (dd, J = 5.6, 3.2 Hz, 2H), 7.16 (t, J = 8.8 Hz, 2H), 4.04 (dd, J = 2.4, 7.2 Hz, 1H), 3.79-3.81 (m, 1H), 3.27 (s, 3H), 2.99-3.04 (m, 1H), 2.73-2.79 (m, 1H), 2.15- 2.17 (m, 2H), 2.06 (s, 2H), 0.94 (s, 9H). MS m/z (ESI): 360.2, M + H+ Purity by HPLC: 99.2% |
| Compound 13 | 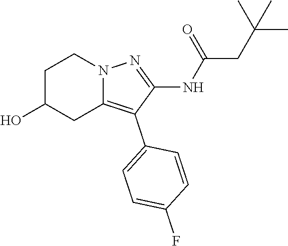 [SMILES: CC(C)(C)CC(=O)Nc1nn2c(c1-c1ccc(F)cc1)CC(O)CC2] | ¹H NMR 400 MHz, DMSO-d₆ δ 9.30 (s, 1H), 7.32 (dd, J = 3.2, 3.6 Hz, 2H), 7.16 (t, J = 8.8 Hz, 2H), 4.08-4.19 (m, 3H), 2.92-2.96 (m, 1H), 2.64- 2.68 (m, 1H), 2.05-2.08 (m, 4H), 0.94 (s, 9H). MS m/z (ESI): 346.2, M + H+ Purity by HPLC: 99.3% |
| Compound 14 | 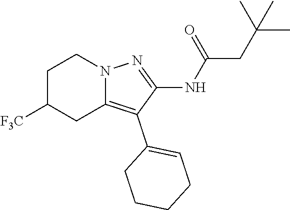 [SMILES: CC(C)(C)CC(=O)Nc1nn2c(c1C1=CCCCC1)CC(C(F)(F)F)CC2] | ¹H NMR 400 MHz, DMSO-d₆ δ 9.21 (s, 1H), 5.50 (s, 1H), 4.13-4.17 (m, 1H), 3.96-3.99 (m, 1H), 2.88-2.96 (m, 2H), 2.65-2.73 (m, 1H), 2.21-2.24 (m, 2H), 2.08 (s, 2H), 1.99- 2.05 (m, 4H), 1.56-1.60 (m, 4H), 1.00 (s, 9H). MS m/z (ESI): 384.3, M + H+ Purity by HPLC: 91.8% |
| Compound 15 | 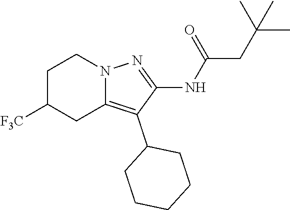 [SMILES: CC(C)(C)CC(=O)Nc1nn2c(c1C1CCCCC1)CC(C(F)(F)F)CC2] | ¹H NMR 400 MHz, DMSO-d₆ δ 9.16 (s, 1H), 4.10-4.14 (m, 1H), 3.97-4.01 (m, 1H), 3.05- 3.09 (m, 2H), 2.66-2.73 (m, 1H), 2.32-2.33 (m, 1H), 2.20- 2.22 (m, 1H), 2.12 (s, 2H), 1.58-1.96 (m, 1H), 1.64-1.73 (m, 5H), 1.42-1.44 (m, 2H), 1.18 (s, 3H), 1.03 (s, 9H). MS m/z (ESI): 386.3, M + H+ Purity by HPLC: 97.5% |
| Compound 16 | 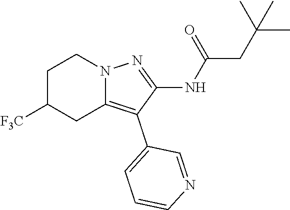 [SMILES: CC(C)(C)CC(=O)Nc1nn2c(c1-c1cccnc1)CC(C(F)(F)F)CC2] | ¹H NMR 400 MHz, DMSO-d₆ δ 9.51 (s, 1H), 8.54 (s, 1H), 8.42-8.43 (m, 1H), 7.70-7.73 (m, 1H), 7.35-7.39 (m, 1H), 4.24-4.28 (m, 1H), 4.05-4.11 (m, 1H), 2.91-3.02 (m, 3H), 2.27-2.30 (m, 1H), 2.03-2.07 (m, 3H), 0.92 (s, 9H). MS m/z (ESI): 381.2, M + H+ Purity by HPLC: 99.2% |
| Compound 17 | 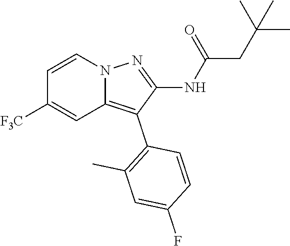 | ¹H NMR 400 MHz, DMSO-d₆ δ 8.98 (s, 1H), 7.07-7.09 (m, 1H), 7.01-7.03 (m, 1H), 6.92- 6.95 (m, 1H), 4.20-4.24 (m, 1H), 4.08-4.12 (m, 1H), 2.96- 2.98 (m, 1H), 2.62-2.68 (m, 2H), 2.29-2.32 (m, 1H), 2.11 (s, 4H), 1.96 (s, 2H), 0.82 (s, 9H). MS m/z (ESI): 412.3, M + H+ Purity by HPLC: 99.2% |
| Compound 18 | 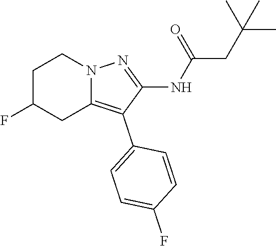 [SMILES: CC(C)(C)CC(=O)Nc1nn2c(c1-c1ccc(F)cc1)CC(F)CC2] | ¹H NMR 400 MHz, DMSO-d₆ δ 9.36 (s, 1H), 7.31-7.34 (m, 2H), 7.14-7.19 (m, 2H), 5.20- 5.33 (m, 1H), 4.05-4.19 (m, 2H), 3.14-3.24 (m, 1H), 2.98- 2.99 (m, 1H), 2.33-2.37 (m, 2H), 2.07 (s, 2H), 0.93 (s, 9H). MS m/z (ESI): 348.1, M + H+ Purity by HPLC: 99.7% |
| Compound 19 | 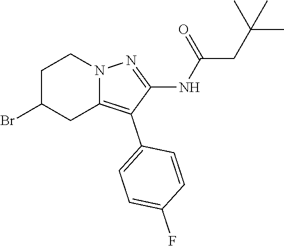 [SMILES: CC(C)(C)CC(=O)Nc1nn2c(c1-c1ccc(F)cc1)CC(Br)CC2] | ¹H NMR 400 MHz, DMSO-d₆ δ 9.36 (s, 1H), 7.31 (dd, J = 5.6, 8.4 Hz, 2H), 7.21-7.12 (m, 2H), 4.90 (d, J = 4.4 Hz, 1H), 4.24-4.11 (m, 2H), 3.56 (dd, J = 4.4, 17.1 Hz, 1H), 3.15 (dd, J = 4.8, 17.1 Hz, 1H), 2.56- 2.51 (m, 1H), 2.45-2.35 (m, 1H), 2.07 (s, 2H), 0.94 (s, 9H). MS m/z (ESI): 407.9, M + H+ Purity by HPLC: 99.7% |
| Compound 20 | 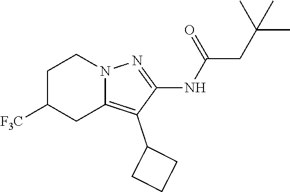 [SMILES: CC(C)(C)CC(=O)Nc1nn2c(c1C1CCC1)CC(C(F)(F)F)CC2] | ¹H NMR 400 MHz, DMSO-d₆ δ 9.23 (s, 1H), 4.11 (dd, J = 3.6, 12.6 Hz, 1H), 3.95 (dt, J = 4.4, 12.2 Hz, 1H), 3.28-3.23 (m, 1H), 3.10-3.02 (m, 1H), 3.01-2.90 (m, 1H), 2.76-2.65 (m, 1H), 2.21 (dd, J = 1.6, 12.7 Hz, 1H), 2.16-2.05 (m 6H), 2.00-1.92 (m, 1H), 1.88- 1.79 (m, 1H), 1.76-1.66 (m, 1H), 1.02 (s, 9H). MS m/z (ESI): 358.1, M + H+ Purity by HPLC: 98.9% |
| Compound 21 | 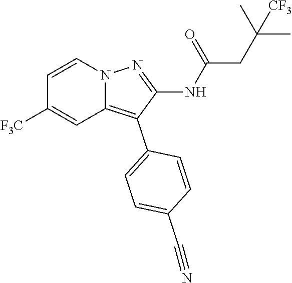 | ¹H NMR 400 MHz, DMSO-d₆ δ 9.88 (s, 1H), 7.82 (d, J = 8.4 Hz, 2H), 7.51 (d, J = 8.0 Hz, 2H), 4.25-4.29 (m, 1H), 4.05- 4.10 (m, 1H), 2.90-3.04 (m, 3H), 2.40 (s, 2H), 2.27-2.30 (m, 1H), 2.07-2.27 (m, 1H), 1.15 (d, J = 4.0 Hz, 6H). MS m/z (ESI): 459.1, M + H+ Purity by HPLC: 100% |
| Compound 22 | 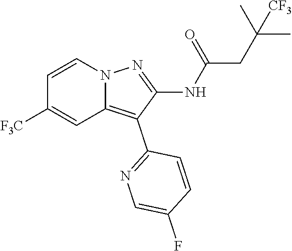 | ¹H NMR 400 MHz, DMSO-d₆ δ 10.0 (s, 1H), 8.57 (d, J = 2.8 Hz, 1H), 7.68-7.32 (m, 1H), 7.48-7.51 (m, 1H), 4.23-4.28 (m, 1H), 4.05-4.12 (m, 1H), 3.30 (d, J = 3.6 Hz, 1H), 2.92- 3.06 (m, 2H), 2.48 (s, 2H), 2.27-2.33 (m, 1H), 2.01-2.11 (m, 1H), 1.21 (s, 6H). MS m/z (ESI): 453.0, M + H+ Purity by HPLC: 99.4% |
| Compound 23 | 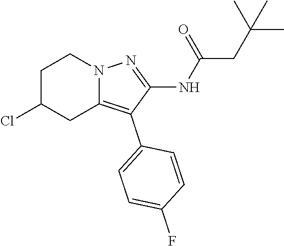 [SMILES: CC(C)(C)CC(=O)Nc1nn2c(c1-c1ccc(F)cc1)CC(Cl)CC2] | ¹H NMR 400 MHz, DMSO-d₆ δ 9.36 (s, 1H), 7.30-7.33 (m, 2H), 7.15-7.20 (m, 2H), 4.80- 4.81 (m, 1H), 4.15-4.21 (m, 2H), 3.38-3.44 (m, 1H), 3.00- 3.05 (m, 1H), 2.45-2.47 (m, 1H), 2.31-2.38 (m, 1H), 2.17 (s, 2H), 0.94 (s, 9H). MS m/z (ESI): 364.0, M + H+ Purity by HPLC: 99.2% |
| Compound 24 | 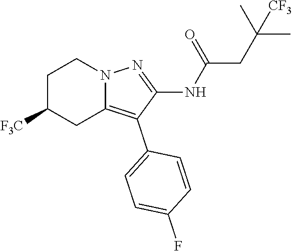 [SMILES: CC(C)(CC(=O)Nc1nn2c(c1-c1ccc(F)cc1)C[C@@H](C(F)(F)F)CC2)C(F)(F)F] | ¹H NMR 400 MHz, DMSO-d₆ δ 9.70 (s, 1H), 7.33-7.36 (m, 2H), 7.17-7.22 (m, 2H), 4.23- 4.27 (m, 1H), 4.05-4.08 (m, 1H), 2.90-32.99 (m, 3H), 2.38 (s, 2H), 2.26-2.30 (m, 1H), 2.06-2.20 (m, 1H), 1.14 (d, J = 2.4 Hz, 6H). MS m/z (ESI): 452.2, M + H⁺ Purity by HPLC: 95.6% SFC retention time: 1.258 min |
| Compound 25 | 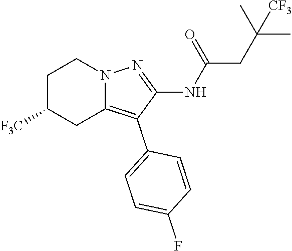 [SMILES: CC(C)(CC(=O)Nc1nn2c(c1-c1ccc(F)cc1)C[C@H](C(F)(F)F)CC2)C(F)(F)F] | ¹H NMR 400 MHz, DMSO-d₆ δ 9.70 (s, 1H), 7.33-7.36 (m, 2H), 7.17-7.22 (m, 2H), 4.23- 4.27 (m, 1H), 4.05-4.08 (m, 1H), 2.90-32.99 (m, 3H), 2.38 (s, 2H), 2.26-2.30 (m, 1H), 2.06-2.20 (m, 1H), 1.14 (d, J = 2.4 Hz, 6H). MS m/z (ESI): 452.1, M + H+ Purity by HPLC: 97.4% SFC retention time: 1.669 min |
| Compound 26 | 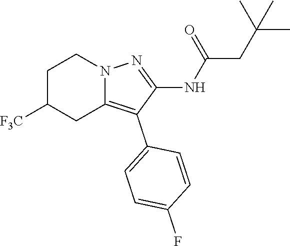 [SMILES: CC(C)(C)CC(=O)Nc1nn2c(c1-c1ccc(F)cc1)CC(C(F)(F)F)CC2] | ¹H NMR 400 MHz, DMSO-d₆ δ 9.42 (s, 1H), 7.35-7.38 (m, 2H), 7.16-7.20 (m, 2H), 4.23- 4.26 (m, 1H), 4.04-4.09 (m, 1H), 2.91-3.01 (m, 3H), 2.28 (d, J = 12.4H, 1H), 2.03-2.08 (m, 3H), 0.94 (s, 9H). MS m/z (ESI): 398.2, M + H⁺ Purity by HPLC: 100% |
| Compound 27 | 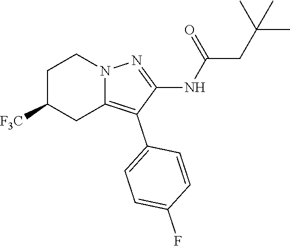 [SMILES: CC(C)(C)CC(=O)Nc1nn2c(c1-c1ccc(F)cc1)C[C@@H](C(F)(F)F)CC2] | ¹H NMR 400 MHz, DMSO-d₆ δ 9.42 (s, 1H), 7.35-7.38 (m, 2H), 7.16-7.20 (m, 2H), 4.23- 4.26 (m, 1H), 4.04-4.09 (m, 1H), 2.91-3.01 (m, 3H), 2.28 (d, J = 12.4H, 1H), 2.03-2.08 (m, 3H), 0.94 (s, 9H). MS m/z (ESI): 398.6, M + H⁺ Purity by HPLC: 99.9% SFC retention time: 1.222 min. |
| Compound 28 | 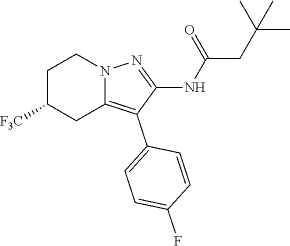 [SMILES: CC(C)(C)CC(=O)Nc1nn2c(c1-c1ccc(F)cc1)C[C@H](C(F)(F)F)CC2] | ¹H NMR 400 MHz, DMSO-d₆ δ 9.42 (s, 1H), 7.35-7.38 (m, 2H), 7.16-7.20 (m, 2H), 4.23- 4.26 (m, 1H), 4.04-4.09 (m, 1H), 2.91-3.01 (m, 3H), 2.28 (d, J = 12.4H, 1H), 2.03-2.08 (m, 3H), 0.94 (s, 9H). MS m/z (ESI): 398.6, M + H⁺ Purity by HPLC: 100% SFC retention time: 1.482 min |
| Compound 29 | 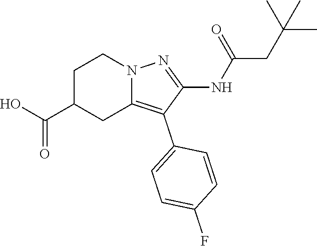 [SMILES: CC(C)(C)CC(=O)Nc1nn2c(c1-c1ccc(F)cc1)CC(C(=O)O)CC2] | ¹H NMR 400 MHz, DMSO-d₆ δ 9.31 (s, 1H), 7.33 (dd, J = 5.6, 8.6 Hz, 2H), 7.16 (t, J = 8.8 Hz, 2H), 4.11 (td, J = 4.8, 12.4 Hz, 1H), 4.06-3.97 (m, 1H), 2.94-2.87 (m, 2H), 2.78- 2.70 (m, 1H), 2.33-2.26 (m, 1H), 2.06 (s, 3H), 0.94 (s, 9H). MS m/z (ESI): 374.1, M + H+ Purity by HPLC: 99.1% |
| Compound 30 | 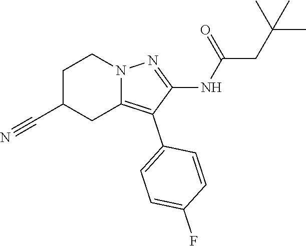 [SMILES: CC(C)(C)CC(=O)Nc1nn2c(c1-c1ccc(F)cc1)CC(C#N)CC2] | ¹H NMR 400 MHz, DMSO-d₆ δ 9.37 (s, 1H), 7.41-7.29 (m, 2H), 7.24-7.10 (m, 2H), 4.13 (t, J = 6.0 Hz, 2H), 3.44 (td, J = 2.8, 5.3 Hz, 1H), 3.18-3.06 (m, 2H), 2.38-2.27 (m, 2H), 2.07 (s, 2H), 0.93 (s, 9H). MS m/z (ESI): 355.1, M + H+ Purity by HPLC: 96.2% |
| Compound 31 | 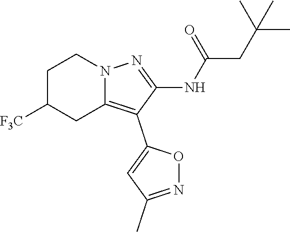 [SMILES: Cc1cc(-c2c(NC(=O)CC(C)(C)C)nn3c2CC(C(F)(F)F)CC3)on1] | ¹H NMR 400 MHz, DMSO-d₆ δ 9.69 (s, 1H), 6.20 (s, 1H), 4.22-4.26 (m, 1H), 4.08-4.12 (m, 1H), 3.21-3.26 (m, 1H), 3.07-3.13 (m, 1H), 2.90-2.97 (m, 1H), 2.27-2.31 (m, 1H), 2.23 (s, 3H), 2.18 (s, 2H), 2.01-2.10 (m, 1H), 1.02 (s, 9H). MS m/z (ESI): 385.2, M + H+ Purity by HPLC: 97.7% |
| Compound 32 | 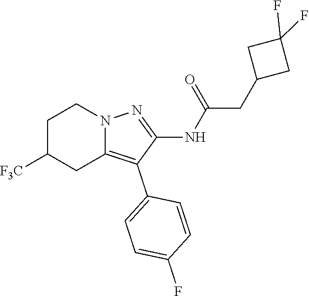 [SMILES: O=C(CC1CC(F)(F)C1)Nc1nn2c(c1-c1ccc(F)cc1)CC(C(F)(F)F)CC2] | ¹H NMR 600 MHz, DMSO-d₆ δ 9.59 (s, 1H), 7.32 (dd, J = 8.5, 5.4 Hz, 2H), 7.21 (t, J = 8.7 Hz, 2H), 4.24 (dd, J = 13.4, 5.1 Hz, 1H), 4.06 (td, J = 12.2, 4.6 Hz, 1H), 3.09-2.85 (m, 3H), 2.68-2.55 (m, 2H), 2.46-2.36 (m, 3H), 2.31- 2.16 (m, 3H), 2.06 (dt, J = 12.4, 6.1 Hz, 1H). MS m/z (ESI): 432.2, M + H+ |
| Compound 33 | 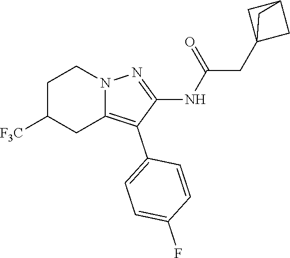 [SMILES: O=C(CC12CC(C1)C2)Nc1nn2c(c1-c1ccc(F)cc1)CC(C(F)(F)F)CC2] | ¹H NMR 600 MHz, DMSO-d₆ δ 9.41 (s, 1H), 7.41-7.32 (m, 2H), 7.19 (t, J = 8.7 Hz, 2H), 4.32-4.18 (m, 1H), 4.06 (td, J = 12.3, 4.7 Hz, 1H), 3.06- 2.83 (m, 3H), 2.69 (s, 2H), 2.34 (s, 2H), 2.05 (dd, J = 12.4, 5.6 Hz, 1H), 1.66 (s, 6H). MS m/z (ESI): 408.2, M + H+ |
| Compound 34 | 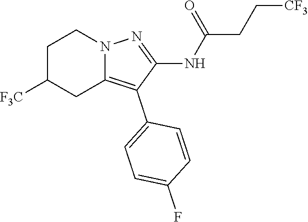 [SMILES: O=C(CCC(F)(F)F)Nc1nn2c(c1-c1ccc(F)cc1)CC(C(F)(F)F)CC2] | ¹H NMR 600 MHz, DMSO-d₆ δ 9.73 (s, 1H), 7.33 (dd, J = 8.5, 5.5 Hz, 2H), 7.19 (t, J = 8.7 Hz, 2H), 4.29-4.20 (m, 1H), 4.06 (td, J = 12.4, 4.7 Hz, 1H), 3.08-2.86 (m, 3H), 2.69 (s, 5H), 2.37-2.27 (m, 1H), 2.06 (dd, J = 12.3, 5.6 Hz, 1H). MS m/z (ESI): 424.2, M + H+ |
| Compound 35 | 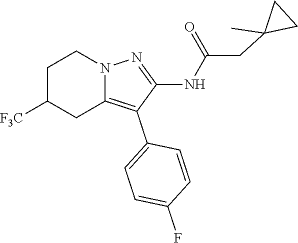 [SMILES: CC1(CC(=O)Nc2nn3c(c2-c2ccc(F)cc2)CC(C(F)(F)F)CC3)CC1] | ¹H NMR 600 MHz, DMSO-d₆ δ 9.36 (s, 1H), 7.36 (dd, J = 8.5, 5.5 Hz, 2H), 7.19 (t, J = 8.8 Hz, 2H), 4.34-4.19 (m, 1H), 4.06 (td, J = 12.4, 4.8 Hz, 1H), 3.05-2.86 (m, 3H), 2.69 (s, 1H), 2.36-2.24 (m, 1H), 2.08 (dd, J = 20.1, 6.3 Hz, 2H), 0.99 (s, 2H), 0.51-0.38 (m, 2H), 0.25 (s, 2H). MS m/z (ESI): 396.2, M + H+ |
| Compound 36 | 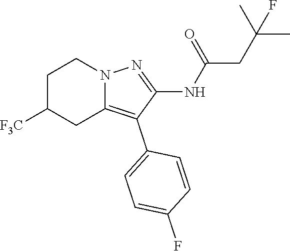 [SMILES: CC(C)(F)CC(=O)Nc1nn2c(c1-c1ccc(F)cc1)CC(C(F)(F)F)CC2] | ¹H NMR 600 MHz, DMSO-d₆ δ 9.63 (s, 1H), 7.35 (dd, J = 8.5, 5.5 Hz, 2H), 7.18 (t, J = 8.7 Hz, 2H), 4.31-4.22 (m, 1H), 4.06 (td, J = 12.4, 4.7 Hz, 1H), 3.07-2.85 (m, 3H), 2.57 (d, J = 16.5 Hz, 2H), 2.28 (dt, J = 10.9, 2.5 Hz, 1H), 2.11- 2.00 (m, 1H), 1.35 (dd, J = 21.8, 9.8 Hz, 6H). MS m/z (ESI): 402.2, M + H+ |
| Compound 37 | 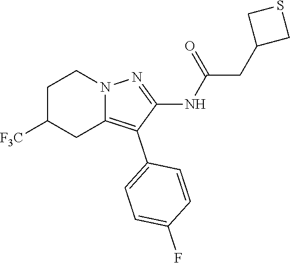 [SMILES: O=C(CC1CSC1)Nc1nn2c(c1-c1ccc(F)cc1)CC(C(F)(F)F)CC2] | ¹H NMR 600 MHz, DMSO-d₆ δ 9.59 (s, 1H), 7.31 (dd, J = 8.4, 5.5 Hz, 2H), 7.21 (t, J = 8.7 Hz, 2H), 4.24 (dd, J = 12.3, 5.3 Hz, 1H), 4.05 (td, J = 12.5, 4.7 Hz, 1H), 3.48 (p, J = 7.9 Hz, 1H), 3.11 (td, J = 8.8, 3.6 Hz, 2H), 3.07-2.87 (m, 6H), 2.34-2.24 (m, 1H), 2.05 (dd, J = 12.0 Hz, 5.4 Hz, 1H), 1.24 (s, 1H). MS m/z (ESI): 414.2, M + H+ |
| Compound 38 | 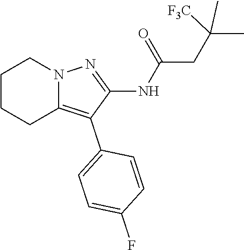 [SMILES: CC(C)(CC(=O)Nc1nn2c(c1-c1ccc(F)cc1)CCCC2)C(F)(F)F] | ¹H NMR 600 MHz, DMSO-d₆ δ 9.65 (s, 1H), 7.44-7.27 (m, 2H), 7.17 (t, J = 8.9 Hz, 2H), 4.03 (s, 2H), 2.75 (s, 2H), 2.38 (s, 2H), 2.11-1.95 (m, 2H), 1.78 (dd, J = 7.7, 4.5 Hz, 2H), 1.15 (s, 6H) MS m/z (ESI): 384.1, M + H+ |
| Compound 39 | 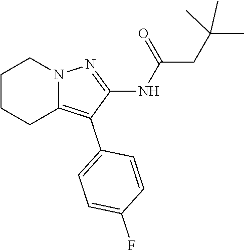 [SMILES: CC(C)(C)CC(=O)Nc1nn2c(c1-c1ccc(F)cc1)CCCC2] | ¹H NMR 600 MHz, DMSO-d₆ δ 9.31 (s, 1H), 7.33 (dd, J = 8.5, 5.6 Hz, 2H), 7.15 (t, J = 8.8 Hz, 2H), 4.02 (t, J = 6.2 Hz, 2H), 2.74 (t, J = 6.3 Hz, 2H), 2.07 (s, 2H), 1.99 (t, J = 4.4 Hz, 2H), 1.78 (dd, J = 6.4, 2.8 Hz, 2H), 0.95 (d, J = 10.8 Hz, 9H). MS m/z (ESI): 330.2, M + H+ |

### Example 3: Synthesis of Compound 40

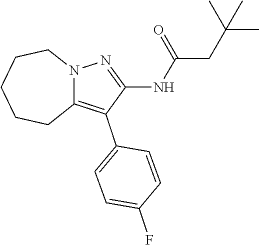 [SMILES: CC(C)(C)CC(=O)Nc1nn2c(c1-c1ccc(F)cc1)CCCCC2]

### Step 1. Preparation of Intermediate 40a

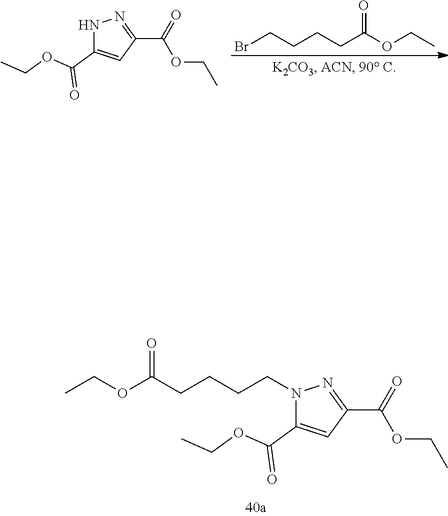

To a solution of compound diethyl 1H-pyrazole-3,5-dicarboxylate (2.00 g, 9.42 mmol, 1.00 eq) in ACN (10.0 mL) was added K₂CO₃ (1.30 g, 9.42 mmol, 1.00 eq) and compound ethyl 5-bromopentanoate (1.97 g, 9.42 mmol, 1.51 mL, 1.00 eq). The mixture was stirred at 90° C. for 2 hrs. LCMS showed compound diethyl 1H-pyrazole-3,5-dicarboxylate was consumed completely and desired mass was detected. The reaction mixture was diluted with H₂O (20.0 mL) and extracted with DCM (20.0 mL×3). The combined organic layers were washed with brine (20.0 mL), dried over Na₂SO₄, filtered and concentrated under reduced pressure to give compound 40a (2.90 g, 8.52 mmol, 90.4% yield) as a yellow solid.

LCMS: Rt=0.398 min, m/z: 341.3, M+H⁺

### Step 2. Preparation of Intermediate 40b

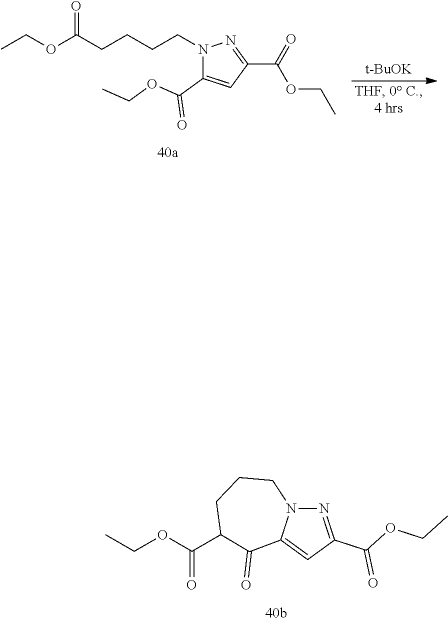

To a solution of compound 40a (2.00 g, 5.88 mmol, 1.00 eq) in THF (20.0 mL) was added t-BuOK (1 M, 5.88 mL, 1.00 eq). The mixture was stirred at 0° C. for 4 hrs. The mixture was quenched by NaHCO₃ (15.00 mL), extracted with DCM (15.00 mL), the organic layer was washed with brine (15.0 mL), dried with Na₂SO₄, filtered and concentrated in vacuum to give the compound 40b (1.73 g, crude) as a white solid.

LCMS: Rt=2.455 min, m/z: 295.1, M+H⁺

### Step 3. Preparation of Intermediate 40c

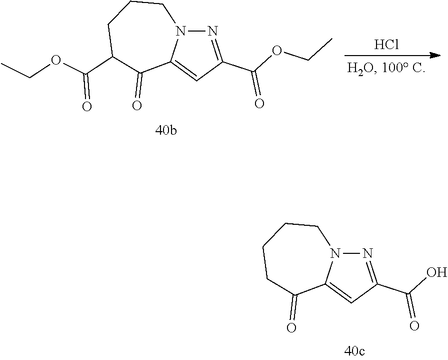 [SMILES: CCOC(=O)c1cc2n(n1)CCCC(C(=O)OCC)C2=O ; Cl ; O=C(O)c1cc2n(n1)CCCCC2=O]

To a solution of compound 40b (1.73 g, 5.88 mmol, 1.00 eq) in H₂O (20.0 mL) was added HCl (12 M, 2.45 mL, 5.00 eq). The mixture was stirred at 100° C. for 12 hrs. The mixture was quenched by NaHCO₃ (20.0 mL), extracted with DCM (20.0×3 mL), the organic layer was washed with brine (15.0 mL), dried with Na₂SO₄, filtered and concentrated in vacuum. Then the residue was purified by column chromatography (SiO₂, Petroleum ether/Ethyl acetate=0/1, R_{f}=0.18) to give the compound 40c (857 mg, 4.41 mmol, 75.0% yield) as a white solid.

LCMS: Rt=0.834 min, m/z: 195.0, M+H⁺

### Step 4. Preparation of Intermediate 40d

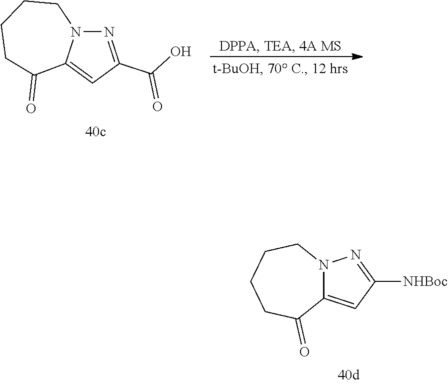 [SMILES: O=C(O)c1cc2n(n1)CCCCC2=O ; "DPPA, TEA, 4A MS" ; CC(C)(C)OC(=O)Nc1cc2n(n1)CCCCC2=O]

To a solution of compound 40c (856 mg, 4.41 mmol, 1.00 eq) in t-BuOH (10.0 mL) was added dropwise TEA (1.56 g, 15.4 mmol, 2.15 mL, 3.50 eq) and DPPA (2.06 g, 7.49 mmol, 1.62 mL, 1.70 eq). After addition, the mixture was stirred at 70° C. for 12 hrs. The mixture was quenched by H₂O (20.0 mL), extracted with DCM (20.0×3 mL), the organic layer was washed with brine (15.0 mL), dried with Na₂SO₄, filtered and concentrated in vacuum. Then the residue was purified by column chromatography (SiO₂, Petroleum ether/Ethyl acetate=0/1, R_{f}=0.45) to give the compound 40d (550 mg, 2.07 mmol, 47.0% yield) as a white solid.

LCMS: Rt=0.341 min, m/z: 210.2, M−56+H⁺

### Step 5. Preparation of Intermediate 40e

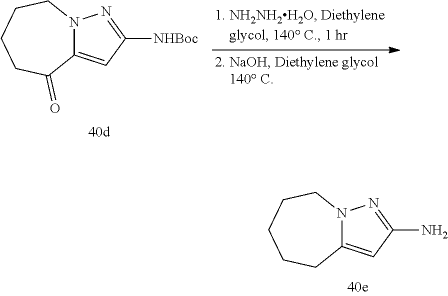 [SMILES: CC(C)(C)OC(=O)Nc1cc2n(n1)CCCCC2=O ; Nc1cc2n(n1)CCCCC2]

To a solution of compound 40d (250 mg, 942 μmol, 1.00 eq) in diethylene glycol (2.50 mL) was added dropwise NH₂NH₂·H₂O (235 mg, 4.71 mmol, 228 μL, 5.00 eq) at 25° C. After addition, the mixture was stirred at 140° C. for 1 hrs, and then KOH (185 mg, 3.30 mmol, 3.5 eq) was added dropwise at 25° C. The resulting mixture was stirred at 140° C. for 2 hrs. The mixture was quenched by H₂O (20.0 mL), extracted with DCM (20.0×3 mL), the organic layer was washed with brine (15.0 mL), dried with Na₂SO₄, filtered and concentrated in vacuum. Then the residue was purified by column chromatography (SiO₂, Petroleum ether/Ethyl acetate=0/1, R_{f}=0.24) to give the compound 40e (250 mg, 1.65 mmol, 87.7% yield) as a white solid.

LCMS: Rt=0.126 min, m/z: 152.2, M+H⁺

### Step 6. Preparation of Intermediate 40f

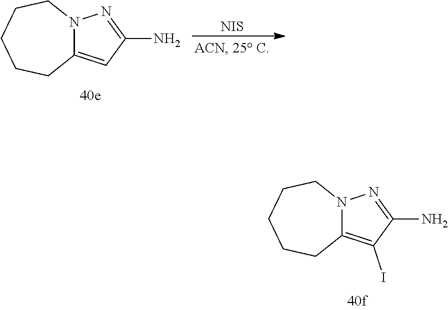

To a solution of compound 40e (200 mg, 1.32 mmol, 1.00 eq) in ACN (2.00 mL) was added NIS (297 mg, 1.32 mmol, 1.00 eq) at 0° C. The mixture was stirred at 25° C. for 1 hrs. The mixture was quenched by H₂O (20.0 mL), extracted with DCM (20.0×3 mL), the organic layer was washed with brine (15.0 mL), dried with Na₂SO₄, filtered and concentrated in vacuum. Then the residue was purified by column chromatography (SiO₂, Petroleum ether/Ethyl acetate=0/1, R_{f}=0.82) to give the compound 40f (180 mg, 649 μmol, 39.3% yield) as a white solid.

LCMS: Rt=0.304 min, m/z: 278.0, M+H⁺

### Step 7. Preparation of Intermediate 40g

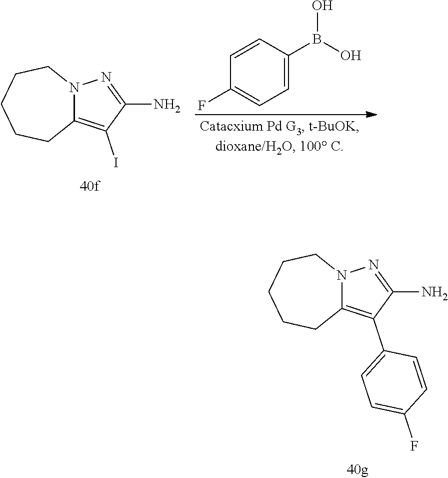

To a solution of compound 40f (170 mg, 613 μmol, 1.00 eq) and compound 4-fluorobenzeneboronic acid (257 mg, 1.84 mmol, 3.00 eq) in H₂O (1.50 mL) and dioxane (1.50 mL) was added Catacxium Pd G₃ (44.6 mg, 61.3 μmol, 0.10 eq) and t-BuONa (117 mg, 1.23 mmol, 2.00 eq) under N₂ atmosphere. The mixture was stirred at 100° C. for 2 hrs. The mixture was quenched by H₂O (20.0 mL), extracted with DCM (20.0×3 mL), the organic layer was washed with brine (15.0 mL), dried with Na₂SO₄, filtered and concentrated in vacuum. Then the residue was purified by column chromatography (SiO₂, Petroleum ether/Ethyl acetate=0/1, R_{f}=0.82) to give the compound 40g (136 mg, 542 μmol, 88.4% yield, 97.9% purity) as a white solid.

LCMS: Rt=0.314 min, m/z: 246.2, M+H⁺

### Step 8. Preparation of Compound 40

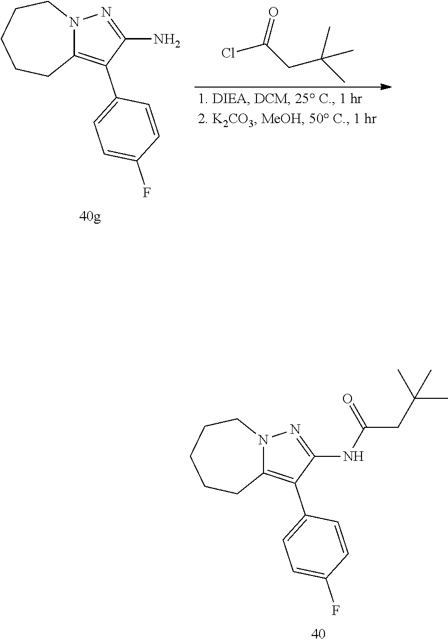

To a solution of compound 40g (136 mg, 554 μmol, 1.00 eq) in DCM (2.00 mL) was added DIEA (143 mg, 1.11 mmol, 193 μL, 2.00 eq) and compound 3,3-dimethylbutanoyl chloride (111 mg, 831 μmol, 115 μL, 1.50 eq). The mixture was stirred at 25° C. for 1 hrs. The reaction mixture was diluted with H₂O (10.0 mL) and extracted with DCM (20.0 mL×3). The combined organic layers were washed with brine (20.0 mL), dried over Na₂SO₄, filtered and concentrated under reduced pressure to give a residue. To a solution of residue in MeOH (5.00 mL) was added K₂CO₃ (152 mg, 1.11 mmol, 2.00 eq). The mixture was stirred at 50° C. for 1 hrs. The mixture was diluted with saturated aqueous solution of H₂O (10.0 mL) and extracted with EtOAc (10.0 mL*3). The combined organic layers were washed with brine (10.0 mL), dried over Na₂SO₄, filtered and concentrated under reduced pressure to give a residue. The residue was purified by prep-HPLC (neutral condition; column: CD02-Waters Xbridge BEH C18 150*25*10 μm; mobile phase: [water (NH₄HCO₃)-ACN]; gradient: 34%-64% B over 10 min) to give compound 40 (160 mg, 465 μmol, 84.2% yield, 99.9% purity) as a white solid.

LCMS: Rt=0.590 min, m/z: 442.3, M+H⁺

LCMS: Rt=2.088 min, m/z: 344.2, M+H⁺

¹H NMR: 400 MHz, DMSO-d₆

¹⁹F NMR: 400 MHz, DMSO-d₆

δ 9.19 (s, 1H), 7.17-7.25 (m, 4H), 4.18-4.21 (m, 2H), 2.67-2.69 (m, 2H), 2.02 (s, 2H), 1.80-1.82 (m, 2H), 1.67-1.69 (m, 2H), 1.57-1.59 (m, 2H), 0.90 (s, 9H).

The preparation of the compounds listed below refers to Example 3

| Name | Structure | Data |
|---|---|---|
| Compound 41 | 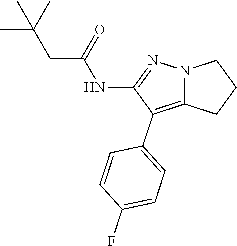 [SMILES: CC(C)(C)CC(=O)Nc1nn2c(c1-c1ccc(F)cc1)CCC2] | ¹H NMR 400 MHz, DMSO-d₆ δ 10.25 (s, 1H), 9.09 (s, 1H), 7.66 (d, J = 8.7 Hz, 2H), 7.55 (d, J = 8.3 Hz, 2H), 4.12 (d, J = 7.6 Hz, 2H), 2.13 (s, 2H), 1.93-1.85 (m, 2H), 1.82- 1.69 (m, 2H), 1.60 (dq, J = 11.1, 8.7 Hz, 2H), 0.96 (s, 1H), 0.88 (s, 9H). MS m/z (ESI): 316.2, M + H+ |
| Compound 42 | 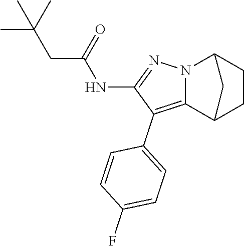 [SMILES: CC(C)(C)CC(=O)Nc1nn2c(c1-c1ccc(F)cc1)C1CCC2C1] | ¹H NMR 400 MHz, DMSO-d₆ δ 9.49 (s, 1H), 7.42-7.45 (m, 2H), 7.16 (t, J = 12.6 Hz, 2H), 4.82 (s, 1H), 3.79 (s, 1H), 2.11 (s, 2H), 1.88-2.06 (m, 3H), 1.82 (t, J = 9.3 Hz, 1H), 1.03- 1.07 (m, 2H), 0.98 (s, 9H). MS m/z (ESI): 342.4, M + H+ Purity by HPLC: 99.9% |

### Example 4: Synthesis of Compound 43

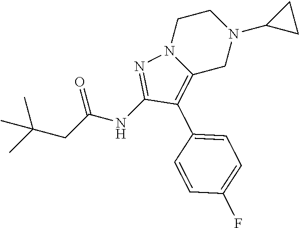 [SMILES: CC(C)(C)CC(=O)Nc1nn2c(c1-c1ccc(F)cc1)CN(C1CC1)CC2]

### Step 1. Preparation of Intermediate 43a

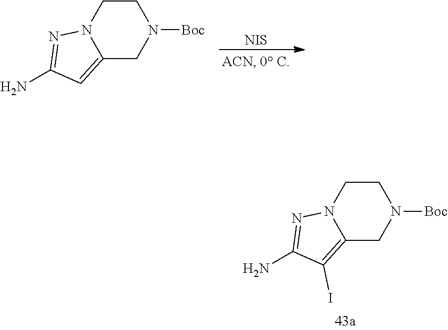

To a solution of compound tert-butyl 2-amino-6,7-dihydropyrazolo[1,5-a]pyrazine-5(4H)-carboxylate (2.00 g, 8.39 mmol, 1.00 eq) in ACN (20.0 mL) was added NIS (1.89 g, 8.39 mmol, 1.00 eq) at 0° C. The mixture was stirred at 0° C. for 2 hrs. TLC (Petroleum ether: Ethyl acetate=1/1, R_{f}=0.38) indicated compound tert-butyl 2-amino-6,7-dihydropyrazolo[1,5-a]pyrazine-5(4H)-carboxylate was consumed completely and one new spot formed. The reaction mixture was diluted with H₂O (100 mL) and extracted with EtOAc (50.0 mL×3). The combined organic layers were washed with brine (50.0 mL), dried over Na₂SO₄, filtered and concentrated under reduced pressure to give a residue. The residue was purified by column chromatography (SiO₂, Petroleum ether/Ethyl acetate=1/1, R_{f}=0.38) to give compound 43a (3.00 g, 8.24 mmol, 98.2% yield) as a yellow solid.

### Step 2. Preparation of Intermediate 43b

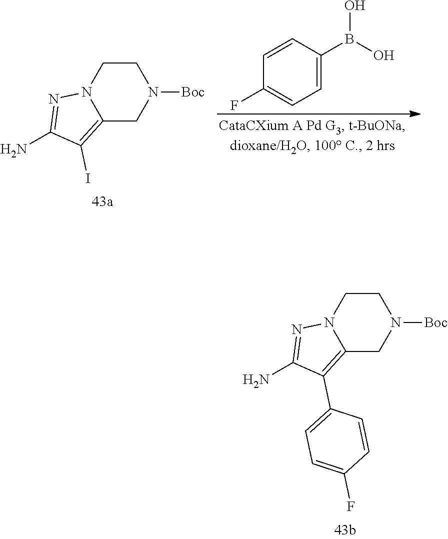 [SMILES: CC(C)(C)OC(=O)N1CCn2nc(N)c(I)c2C1 ; OB(O)c1ccc(F)cc1 ; CC(C)(C)OC(=O)N1CCn2nc(N)c(-c3ccc(F)cc3)c2C1]

To a solution of compound 43a (3.00 g, 8.24 mmol, 1.00 eq) and compound 4-fluorobenzeneboronic acid (3.46 g, 24.7 mmol, 3.00 eq) in dioxane (30.0 mL) and H₂O (10.0 mL) was added CataCXium A Pd G3 (599 mg, 823 μmol, 0.10 eq) and t-BuONa (1.58 g, 16.4 mmol, 2.00 eq). The mixture was stirred at 100° C. for 2 hrs under N₂ atmosphere. LCMS showed compound 43a was consumed completely and desired mass was detected. The reaction mixture was diluted with H₂O (50.0 mL) and extracted with EtOAc (30.0 mL×3). The combined organic layers were washed with brine (30.0 mL), dried over Na₂SO₄, filtered and concentrated under reduced pressure to give a residue. The residue was purified by column chromatography (SiO₂, Petroleum ether/Ethyl acetate=0/1, R_{f}=0.14) to give compound 43b (2.50 g, 7.52 mmol, 91.3% yield, 100% purity) as a yellow solid.

LCMS: Rt=0.399 min, m/z: 333.2, M+H⁺

LCMS: Rt=0.387 min, m/z: 333.1, M+H⁺

### Step 3. Preparation of Intermediate 43c

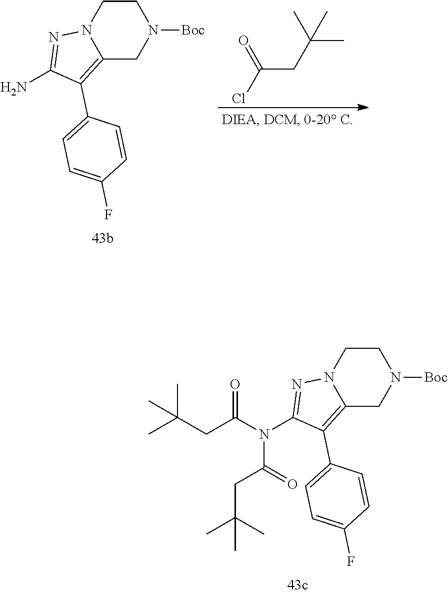

To a solution of compound 43b (2.50 g, 7.52 mmol, 1.00 eq) in DCM (25.0 mL) was added DIEA (1.94 g, 15.0 mmol, 2.62 mL, 2.00 eq) and compound 3,3-dimethylbutanoyl chloride (2.02 g, 15.0 mmol, 2.09 mL, 2.00 eq) at 0° C. The mixture was stirred at 20° C. for 2 hrs. LCMS showed compound 43b was consumed completely and the desired mass was detected. The reaction mixture was diluted with H₂O (50.0 mL) and extracted with DCM (20.0 mL×3). The combined organic layers were washed with brine (20.0 mL), dried over Na₂SO₄, filtered and concentrated under reduced pressure to give compound 43c (4.00 g, crude) as a yellow oil.

LCMS: m/z: 529.7, M+H⁺

### Step 4. Preparation of Intermediate 43d

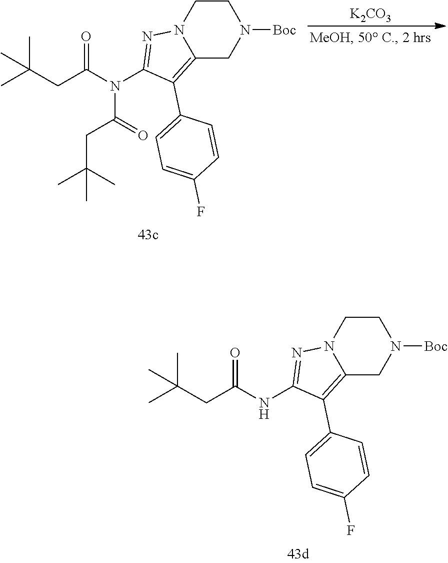

To a solution of compound 43c (4.00 g, 7.57 mmol, 1.00 eq) in MeOH (40.0 mL) was added K₂CO₃ (1.57 g, 11.3 mmol, 1.50 eq). The mixture was stirred at 50° C. for 2 hrs. LCMS showed compound 43c was consumed completely and desired mass was detected. The reaction mixture was diluted with H₂O (100 mL) and extracted with DCM (50.0 mL×3). The combined organic layers were washed with brine (50.0 mL), dried over Na₂SO₄, filtered and concentrated under reduced pressure to give a residue. The residue was purified by column chromatography (SiO₂, Petroleum ether/Ethyl acetate=10/1 to 0/1, R_{f}=0.51) to give compound 43d (3.00 g, 6.96 mmol, 92.0% yield) as a yellow solid.

LCMS: Rt=0.462 min, m/z: 431.7, M+H⁺

### Step 5. Preparation of Compound 46

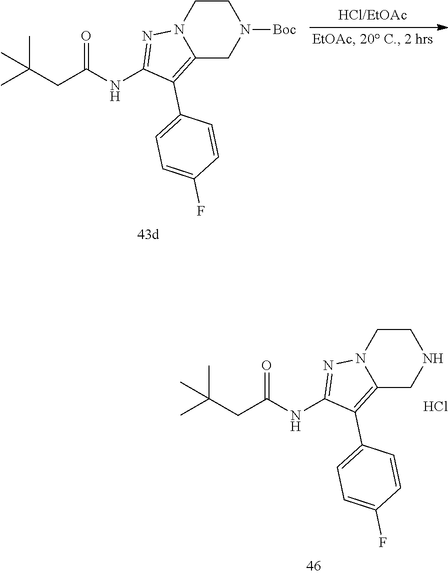 [SMILES: CC(C)(C)CC(=O)Nc1nn2c(c1-c1ccc(F)cc1)CN(C(=O)OC(C)(C)C)CC2 ; "HCl/EtOAc" ; CC(C)(C)CC(=O)Nc1nn2c(c1-c1ccc(F)cc1)CNCC2 ; Cl]

To a solution of compound 43d (3.00 g, 6.96 mmol, 92.0% yield) in EtOAc (10.0 mL) was added HCl/EtOAc (2 M, 20.0 mL, 5.00 eq). The mixture was stirred at 20° C. for 2 hrs. LCMS showed compound 43d was consumed completely and desired mass was detected. The reaction mixture was concentrated under reduced pressure to give the compound 46 (2.69 g, 8.14 mmol, 100% yield) as a light-yellow solid.

LCMS: Rt=0.277 min, m/z: 331.2, M+H⁺

### Step 6. Preparation of Compound 43

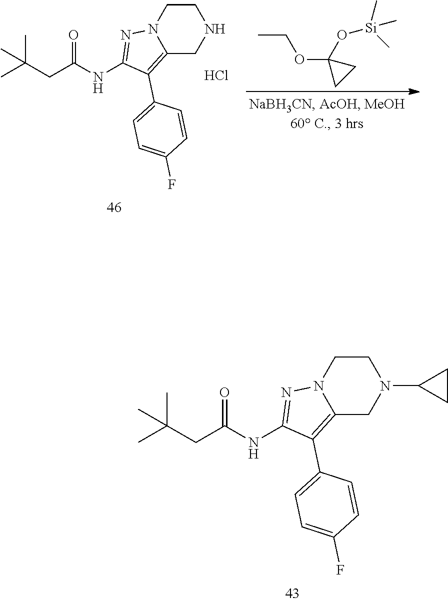 [SMILES: CC(C)(C)CC(=O)Nc1nn2c(c1-c1ccc(F)cc1)CNCC2 ; Cl ; CCOC1(O[Si](C)(C)C)CC1 ; CC(C)(C)CC(=O)Nc1nn2c(c1-c1ccc(F)cc1)CN(C1CC1)CC2]

To a solution of compound 46 (2.69 g, 8.14 mmol, 1.00 eq) in MeOH (2.00 mL), compound (1-ethoxycyclopropoxy)trimethylsilane (8.52 g, 48.8 mmol, 9.82 mL, 6.00 eq), NaBH₃CN (2.05 g, 32.5 mmol, 4.00 eq) and AcOH (4.89 g, 81.4 mmol, 4.66 mL, 10.0 eq) was added. The mixture was stirred at 60° C. for 3 hrs. LCMS showed compound 46 was consumed completely and desired mass was detected. The reaction mixture was diluted with H₂O (50.0 mL) and extracted with DCM (20.0 mL×3). The combined organic layers were washed with brine (20.0 mL), dried over Na₂SO₄, filtered and concentrated under reduced pressure to give a residue. The residue was purified by column chromatography (SiO₂, Petroleum ether/Ethyl acetate=0/1, R_{f}=0.27) to give compound 43 (2.20 g, 6.42 mmol, 69.4% yield, 95.2% purity) as a white solid.

LCMS: m/z=371.2, M+H⁺

¹H NMR: 400 MHz, DMSO-d₆ δ 9.37 (s, 1H), 7.29-7.33 (m, 2H), 7.16-7.20 (m, 2H), 4.03 (t, J=5.2 Hz, 2H), 3.78 (s, 2H), 3.11 (t, J=5.6 Hz, 2H), 2.08 (s, 2H), 1.93-1.96 (m, 1H), 0.95 (s, 9H), 0.48-0.52 (m, 2H), 0.40-0.42 (m, 2H).

The preparation of the compounds listed below refers to Example 4

| Name | Structure | Data |
|---|---|---|
| Compound 44 | 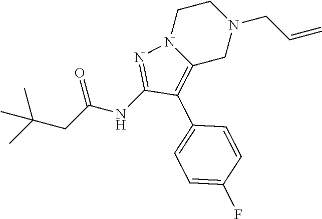 [SMILES: C=CCN1CCn2nc(NC(=O)CC(C)(C)C)c(-c3ccc(F)cc3)c2C1] | ¹H NMR 400 MHz, DMSO-d₆ δ 9.38 (s, 1H), 7.27-7.30 (m, 2H), 7.15-7.19 (m, 2H), 7.81- 7.88 (m, 1H), 5.23-5.28 (m, 1H), 5.16-5.19 (m, 1H), 4.06 (t, J = 5.2 Hz, 2H), 3.63 (s, 2H), 3.20 (d, J = 6.4 Hz, 2H), 2.93 (t, J = 5.6 Hz, 2H), 2.08 (s, 2H), 0.94 (s, 9H). MS m/z (ESI): 371.2, M + H+ Purity by HPLC: 99.6% |
| Compound 45 | 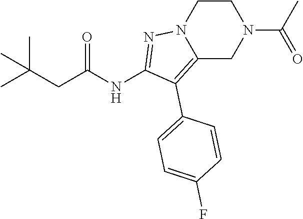 [SMILES: CC(=O)N1CCn2nc(NC(=O)CC(C)(C)C)c(-c3ccc(F)cc3)c2C1] | ¹H NMR 400 MHz, DMSO-d₆ δ 9.43 (d, J = 11.2 Hz, 1H), 7.39 (dd, J = 8.4, 5.5 Hz, 1H), 7.37-7.29 (m, 1H), 7.20 (td, J = 8.4, 5.4 Hz, 2H), 4.73 (d, J = 41.8 Hz, 2H), 4.16 (t, J = 5.5 Hz, 1H), 4.05 (d, J = 5.6 Hz, 1H), 4.04-3.90 (m, 2H), 2.18- 2.05 (m, 5H), 0.94 (d, J = 2.2 Hz, 9H). MS m/z (ESI): 373.2, M + H+ |
| Compound 46 | 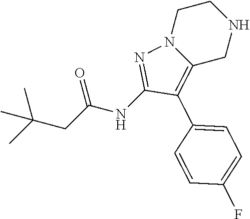 [SMILES: CC(C)(C)CC(=O)Nc1nn2c(c1-c1ccc(F)cc1)CNCC2] | MS m/z (ESI): 331.2 M + H+ |
| Compound 47 | 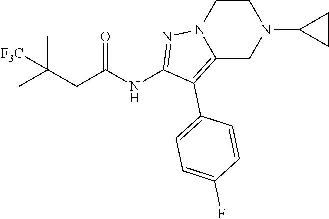 [SMILES: CC(C)(CC(=O)Nc1nn2c(c1-c1ccc(F)cc1)CN(C1CC1)CC2)C(F)(F)F] | ¹H NMR 400 MHz, DMSO-d₆ δ 9.71 (s, 1H), 7.28-7.31 (m, 2H), 7.17-7.21 (m, 2H), 4.03 (t, J = 5.6 Hz, 2H), 3.78 (s, 2H), 3.10 (t, J = 5.6 Hz, 2H), 2.39 (s, 2H), 1.93-1.96 (m, 1H), 1.15 (s, 9H), 0.47-0.49 (m, 2H), 0.39-0.41 (m, 2H). MS m/z (ESI): 425.2, M + H+ Purity by HPLC: 98.9% |
| Compound 48 | 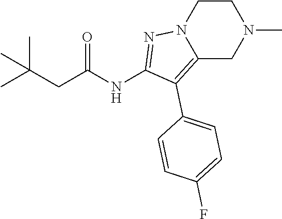 [SMILES: CN1CCn2nc(NC(=O)CC(C)(C)C)c(-c3ccc(F)cc3)c2C1] | ¹H NMR 400 MHz, DMSO-d₆ δ 9.11 (s, 1H), 7.61 (s, 1H), 7.41 (dd, J = 8.6, 5.7 Hz, 2H), 7.25 (t, J = 8.9 Hz, 2H), 3.92 (d, J = 7.5 Hz, 2H), 2.38 (p, J = 7.7 Hz, 1H), 1.98 (s, 3H), 1.88-1.78 (m, 2H), 1.80- 1.66 (m, 3H), 1.60-1.51 (m, 2H), 0.86 (s, 9H). MS m/z (ESI): 344.3, M + H+ |
| Compound 49 | 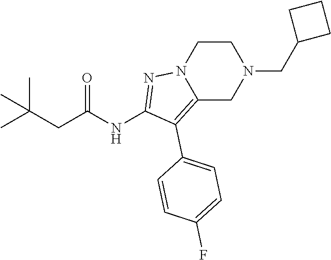 | ¹H NMR 400 MHz, DMSO-d₆ δ 9.69 (s, 1H), 8.00 (s, 1H), 7.74 (dd, J = 8.7, 5.8 Hz, 2H) 7.19 (t, J = 8.9 Hz, 2H), 4.37 (d, J = 4.6 Hz, 1H), 3.12 (dt, J = 14.7, 8.9 Hz, 4H), 2.29 (s, 2H), 1.04 (s, 9H). MS m/z (ESI): 366.2, M + H+ |
| Compound 50 | 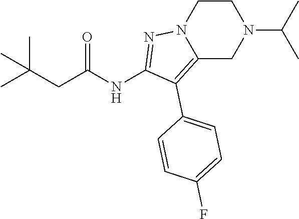 [SMILES: CC(C)N1CCn2nc(NC(=O)CC(C)(C)C)c(-c3ccc(F)cc3)c2C1] | ¹H NMR 400 MHz, DMSO-d₆ δ 9.39 (s, 1H), 7.32 (dd, J = 8.4, 5.6 Hz, 2H), 7.17 (t, J = 8.8 Hz, 2H), 4.02 (s, 2H), 3.69 (s, 2H), 3.03-2.84 (m, 3H), 2.08 (s, 2H), 1.04 (d, J = 6.5 Hz, 6H), 0.94 (s, 9H). MS m/z (ESI): 373.2, M + H+ |
| Compound 51 | 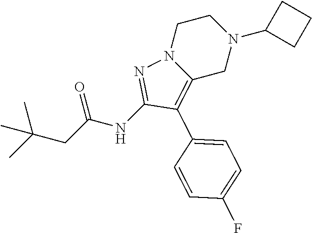 [SMILES: CC(C)(C)CC(=O)Nc1nn2c(c1-c1ccc(F)cc1)CN(C1CCC1)CC2] | ¹H NMR 400 MHz, DMSO-d₆ δ 9.39 (s, 1H), 7.29 (dd, J = 8.5, 5.5 Hz, 2H), 7.17 (t, J = 8.7 Hz, 2H), 4.04 (s, 2H), 3.50 (s, 2H), 3.00 (t, J = 7.8 Hz, 1H), 2.80 (t, J = 5.6 Hz, 2H), 2.07 (s, 2H), 2.03 (q, J = 7.8, 7.4 Hz, 2H), 1.86-1.77 (m, 2H), 1.71-1.56 (m, 2H), 0.94 (s, 9H). MS m/z (ESI): 385.3, M + H+ |
| Compound 52 | 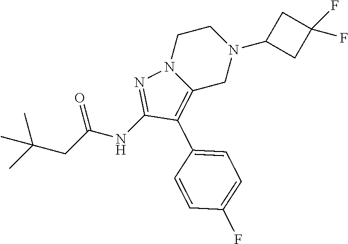 [SMILES: CC(C)(C)CC(=O)Nc1nn2c(c1-c1ccc(F)cc1)CN(C1CC(F)(F)C1)CC2] | ¹H NMR 400 MHz, DMSO-d₆ δ 9.39 (s, 1H), 7.31 (dd, J = 8.5, 5.5 Hz, 2H), 7.17 (t, J = 8.7 Hz, 2H), 4.07 (s, 2H), 3.61 (s, 2H), 3.07-2.94 (m, 1H), 2.88 (t, J = 5.6 Hz, 2H), 2.76 (d, J = 9.5 Hz, 2H), 2.07 (s, 2H), 1.24 (s, 2H), 0.94 (s, 9H). MS m/z (ESI): 421.2, M + H+ |

### Example 5: Synthesis of Compound 53

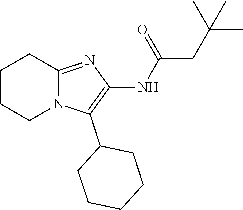 [SMILES: CC(C)(C)CC(=O)Nc1nc2n(c1C1CCCCC1)CCCC2]

### Step 1. Preparation of Compound 53

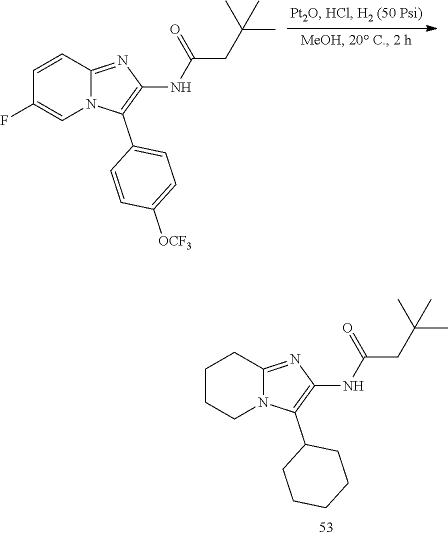 [SMILES: CC(C)(C)CC(=O)Nc1nc2ccc(F)cn2c1-c1ccc(OC(F)(F)F)cc1 ; CC(C)(C)CC(=O)Nc1nc2n(c1C1CCCCC1)CCCC2]

To a solution of N-(6-fluoro-3-(4-(trifluoromethoxy)phenyl)imidazo[1,2-a]pyridin-2-yl)-3,3-dimethylbutanamide (50.0 mg, 122 μmol, 1.00 eq) in MeOH (5.00 mL) was added PtO₂ (27.7 mg, 122 μmol, 1.00 eq) and HCl (12 M, 10.1 μL, 1.00 eq) under N₂ atmosphere. The suspension was degassed and purged with H₂ for 3 times. The mixture was stirred under H₂ (50 Psi) at 20° C. The mixture was stirred at 20° C. for 2 hours. The reaction mixture was filtered and concentrated under reduced pressure to give a residue. The residue was purified by preparative-HPLC (neutral condition; column: CD02-Waters Xbridge BEHC18 150*25*10 μm; mobile phase: [water (NH₄HCO₃)-ACN]; gradient:28%-58% B over 10 min) to give compound 53 (14.0 mg, 43.1 μmol, 35.3% yield, 97.9% purity) as a white solid.

LCMS: EC6665-1394-P1A4. Rt=0.337 min, m/z: 318.3, M+H⁺

HPLC: Rt=2.200 min

¹H NMR: 400 MHz, DMSO-d₆ δ10.17 (s, 1H), 8.48 (s, 1H), 7.81-7.83 (m, 1H), 7.63-7.66 (m, 1H), 7.37-7.39 (m, 1H), 7.27-7.29 (m, 2H), 3.22 (s, 4H), 2.20 (s, 2H), 0.98 (s, 9H).

The preparation of the compounds listed below refers to Example 5

| Name | Structure | Data |
|---|---|---|
| Compound 54 | 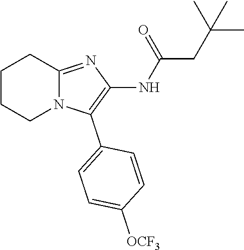 [SMILES: CC(C)(C)CC(=O)Nc1nc2n(c1-c1ccc(OC(F)(F)F)cc1)CCCC2] | MS m/z (ESI): 396.2, M + H⁺ Purity by HPLC: 99.6% |

### Example 6: Synthesis of Compound 55

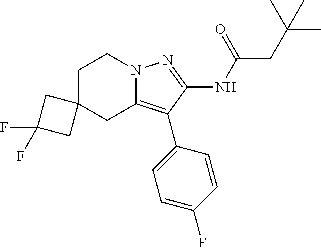 [SMILES: CC(C)(C)CC(=O)Nc1nn2c(c1-c1ccc(F)cc1)CC1(CC2)CC(F)(F)C1]

### Step 1. Preparation of Intermediate 55a

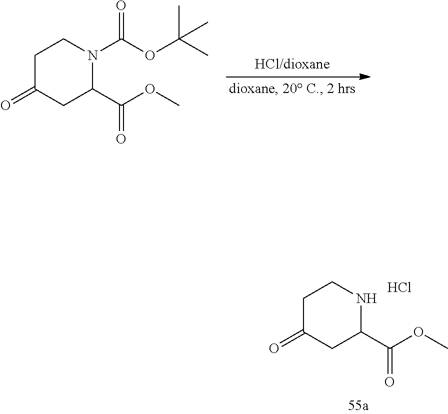 [SMILES: COC(=O)C1CC(=O)CCN1C(=O)OC(C)(C)C ; COC(=O)C1CC(=O)CCN1 ; Cl]

To a solution of compound 1-(tert-butyl) 2-methyl 4-oxopiperidine-1,2-dicarboxylate (15.0 g, 58.3 mmol, 1.00 eq) in dioxane (20.0 mL) was added HCl/dioxane (2 M, 29.1 mL, 1.00 eq). The mixture was stirred at 20° C. for 2 hrs. The reaction mixture was concentrated under reduced pressure to give the compound 55a (11.5 g, crude, HCl) as a white solid.

LCMS: m/z: 158.3, M+H⁺

### Step 2. Preparation of Intermediate 55b

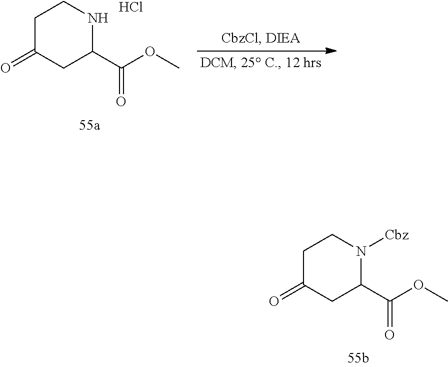 [SMILES: COC(=O)C1CC(=O)CCN1 ; Cl ; COC(=O)C1CC(=O)CCN1C(=O)OCc1ccccc1]

To a solution of compound 55a (11.5 g, 59.3 mmol, 1.00 eq, HCl) in DCM (110 mL) was added DIEA (15.3 g, 118 mmol, 20.6 mL, 2.00 eq) and CbzCl (11.1 g, 65.3 mmol, 9.33 mL, 1.10 eq). The mixture was stirred at 25° C. for 12 hrs. The reaction mixture was extracted with DCM (50.0 mL). The combined organic layers were washed with brine (30.0 mL×2), dried over Na₂SO₄, filtered and concentrated under reduced pressure to give a residue. The residue was purified by column chromatography (SiO₂, Petroleum ether/EtOAc=2/1, Rf=0.33) to give compound 55b (14.0 g, 47.4 mmol, 79.9% yield, 98.8% purity) as a white solid.

LCMS: m/z: 292.2, M+H⁺

### Step 3. Preparation of Intermediate 55c

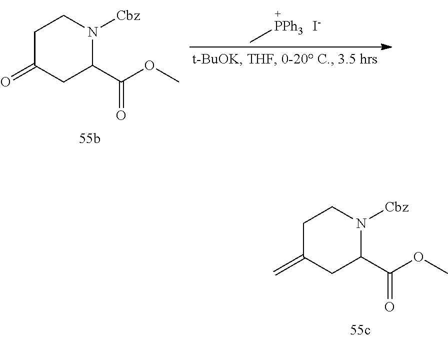

To a solution of methyl (triphenyl)phosphonium iodide (18.9 g, 52.8 mmol, 1.10 eq) in THF (100 mL) was added dropwise t-BuOK (1 M, 50.4 mL, 1.05 eq) at 0° C. After addition, the mixture was stirred at 0° C. for 0.5 hrs, and then compound 55b (14.0 g, 48.0 mmol, 1.00 eq) was added dropwise at 0° C. The resulting mixture was stirred at 20° C. for 3 hrs. The reaction mixture was then diluted in water and extracted with EtOAC (5×50.0 mL). The combined ether layers were washed with brine (2×50.0 mL), dried over magnesium sulfate, filtered, and concentrated under reduced pressure to give a residue. The residue was purified by column chromatography (SiO₂, Petroleum ether/Ethyl acetate=2/1, Rf=0.72) to give the compound 55c (8.30 g, 26.8 mmol, 55.8% yield, 93.6% purity) as a white solid.

LCMS: m/z: 290.2, M+H⁺

### Step 4. Preparation of Intermediate 55d

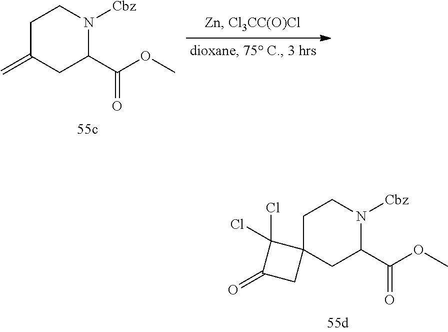

To a solution of compound 55c (5.00 g, 17.2 mmol, 1.00 eq) in dioxane (50.0 mL) was degassed and purged with N₂ for 3 times, and was added Zn (3.39 g, 51.8 mmol, 3.00 eq) under N₂ atmosphere in batch addition, and followed by batch addition of 2,2,2-trichloroacetyl chloride (4.08 g, 22.4 mmol, 2.50 mL, 1.30 eq) at 75° C. Then the mixture was stirred at 75° C. for 3 hrs under N₂ atmosphere. The reaction mixture was diluted with aqueous solution of NaHCO₃ (30.0 mL) and extracted with EtOAc (20.0 mL×3). The combined organic layers were washed with brine (30.0 mL), dried over Na₂SO₄, filtered and concentrated under reduced pressure to give compound 55d (6.90 g, crude) as a white solid.

### Step 5. Preparation of Intermediate 55e

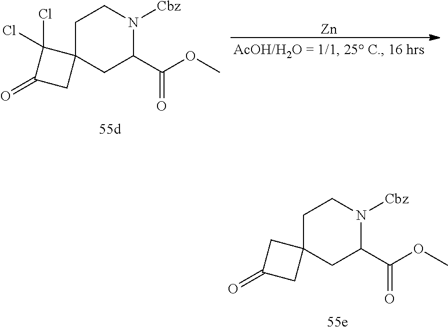

To a solution of compound 55d (6.90 g, 17.2 mmol, 1.00 eq) in AcOH (20 mL) and H2O (20.0 mL) was degassed and purged with N2 for 3 times, and was added Zn (3.49 g, 53.4 mmol, 3.10 eq) under N₂ atmosphere in batch addition at 0° C. The mixture was stirred at 25° C. for 16 hrs. The reaction mixture was diluted with H₂O (30.0 mL) and extracted with EtOAc (30.0 mL×3). The combined organic layers were washed with brine (30.0 mL), dried over Na₂SO₄, filtered and concentrated under reduced pressure to give a residue. The residue was purified by column chromatography (SiO₂, Petroleum ether/Ethyl acetate=2/1, Rf=0.39) to give compound 55e (3.50 g, 7.69 mmol, 44.6% yield, 72.8% purity) as a yellow solid.

LCMS: m/z: 332.1, M+H+

### Step 6. Preparation of Intermediate 55f

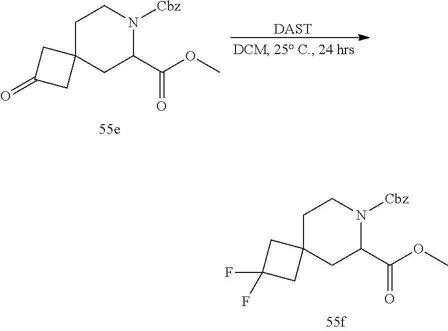 [SMILES: COC(=O)C1CC2(CCN1C(=O)OCc1ccccc1)CC(=O)C2 ; "DAST" ; COC(=O)C1CC2(CCN1C(=O)OCc1ccccc1)CC(F)(F)C2]

To a solution of compound 55e (3.50 g, 10.5 mmol, 1.00 eq) in DCM (35.0 mL) was added DAST (17.0 g, 105 mmol, 13.9 mL, 10.0 eq) at 0° C., The mixture was stirred at 25° C. for 24 hrs. The reaction mixture was diluted with H₂O (20.0 mL) and extracted with EtOAc (20.0 mL×3). The combined organic layers were washed with brine (10.0 mL), dried over Na₂SO₄, filtered and concentrated under reduced pressure to give a residue. The residue was purified by column chromatography (SiO₂, Petroleum ether/Ethyl acetate=50/1 to 2/1, Rf=0.65) to give compound 55f (2.81 g, 7.67 mmol, 72.6% yield, 96.5% purity) as a yellow solid.

LCMS: m/z: 354.1, M+H+

### Step 7. Preparation of Intermediate 55g

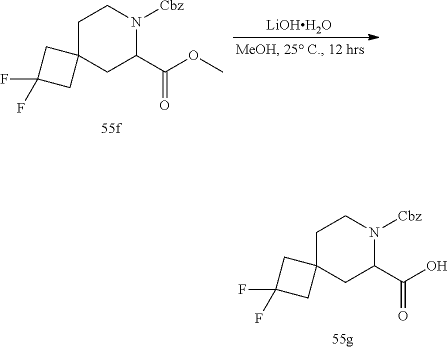 [SMILES: COC(=O)C1CC2(CCN1C(=O)OCc1ccccc1)CC(F)(F)C2 ; "LiOH·H2O" ; O=C(O)C1CC2(CCN1C(=O)OCc1ccccc1)CC(F)(F)C2]

To a solution of compound 55f (2.81 g, 7.95 mmol, 1.00 eq) in MeOH (30.0 mL) was added LiOH·H₂O (667 mg, 15.9 mmol, 2.00 eq). The mixture was stirred at 25° C. for 12 hrs. The reaction mixture was diluted with H₂O (50.0 mL) and extracted with EtOAc (30.0 mL×3). The combined organic layers were washed with brine (30.0 mL), dried over Na₂SO₄, filtered and concentrated under reduced pressure to give compound 55g (2.70 g, 7.96 mmol, 100% yield) as a yellow solid.

LCMS: m/z: 340.1, M+H⁺

### Step 8. Preparation of Intermediate 55h

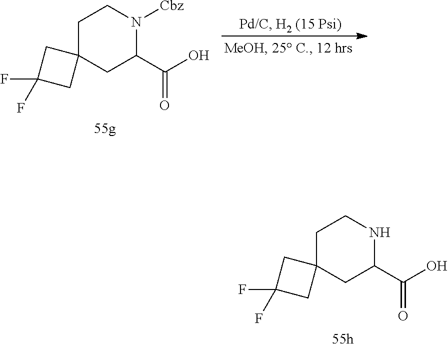 [SMILES: O=C(O)C1CC2(CCN1C(=O)OCc1ccccc1)CC(F)(F)C2 ; "Pd/C, H2 (15 Psi)" ; O=C(O)C1CC2(CCN1)CC(F)(F)C2]

To a solution of compound 55g (2.70 g, 7.96 mmol, 1.00 eq) in MeOH (25.0 mL) was added Pd/C (846 mg, 795 μmol, 10% purity, 0.1 eq) under N2 atmosphere. The suspension was degassed and purged with H₂ for 3 times. The mixture was stirred under H2 (15 Psi or atm.) at 25° C. for 12 hrs. The reaction mixture was filtered and concentrated under reduced pressure to give compound 55h (1.63 g, 7.94 mmol, 100% yield) as a white solid.

LCMS: m/z: 206.1, M+H⁺

### Step 9. Preparation of Intermediate 55i

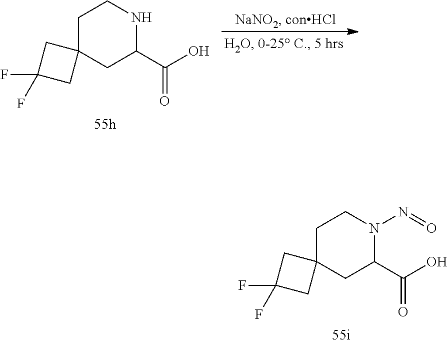 [SMILES: O=C(O)C1CC2(CCN1)CC(F)(F)C2 ; "NaNO2, con·HCl" ; O=NN1CCC2(CC1C(=O)O)CC(F)(F)C2]

To a solution of compound 55h (1.63 g, 7.94 mmol, 1.00 eq) in H2O (20.0 mL) was added NaNO2 (1.10 g, 15.8 mmol, 2.00 eq) and HCl (12 M, 661 μL, 1.00 eq) at 0° C. The mixture was stirred at 25° C. for 5 hrs. The reaction mixture was diluted with H2O (10.0 mL) and extracted with DCM (10.0 mL×3). The combined organic layers were washed with brine (10.0 mL), dried over Na2SO4, filtered and concentrated under reduced pressure to give compound 55i (1.86 g, 7.94 mmol, 100% yield) as a light yellow solid.

LCMS: m/z: 235.0, M+H⁺

### Step 10. Preparation of Intermediate 55j

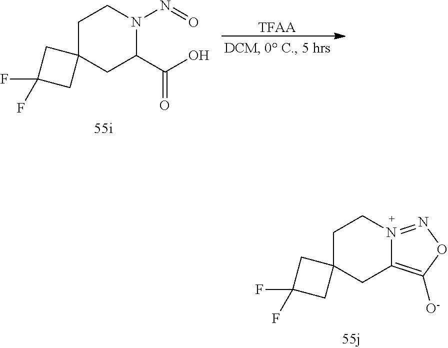

T To a solution of compound 55i (1.86 g, 7.94 mmol, 1.00 eq) in DCM (20.0 mL) was added TFAA (2.17 g, 10.3 mmol, 1.44 mL, 1.30 eq). The mixture was stirred at 0° C. for 5 hrs. The reaction mixture was concentrated under reduced pressure to give a residue. The residue was triturated with MTBE (10.0 ml) at 0° C. for 10 min to give compound 55j (1.40 g, 6.48 mmol, 81.5% yield) as a white solid.

LCMS: m/z: 217.0, M+H⁺

### Step 11. Preparation of Intermediate 55k

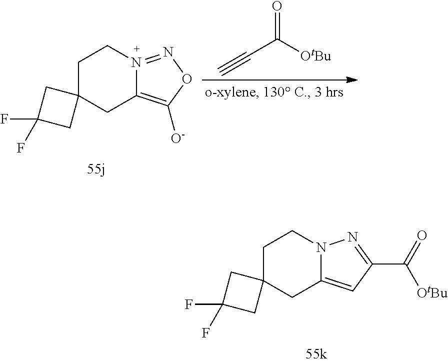

To a solution of compound 55j (1.40 g, 6.48 mmol, 1.00 eq) in o-xylene (15.0 mL) was added tert-butyl prop-2-ynoate (8.17 g, 64.7 mmol, 8.89 mL, 10.0 eq). The mixture was stirred at 130° C. for 3 hrs. The reaction mixture was diluted with H₂O (30.0 mL) and extracted with EtOAc (30.0 mL×3). The combined organic layers were washed with brine (30.0 mL), dried over Na₂SO₄, filtered and concentrated under reduced pressure to give a residue. The residue was purified by column chromatography (SiO₂, Petroleum ether/Ethyl acetate=2/1, Rf=0.43) to give the compound 55k (1.80 g, 6.03 mmol, 74.5% yield, 100% purity) as a yellow solid.

LCMS: m/z: 243.0, M−56+H⁺

### Step 12. Preparation of Intermediate 55l

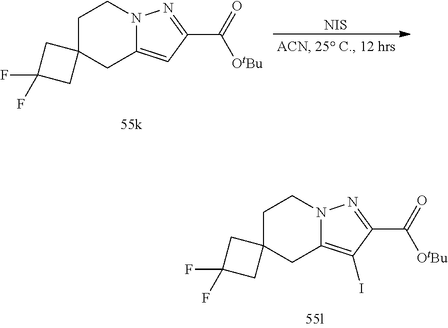 [SMILES: CC(C)(C)OC(=O)c1cc2n(n1)CCC1(C2)CC(F)(F)C1 ; "NIS" ; CC(C)(C)OC(=O)c1nn2c(c1I)CC1(CC2)CC(F)(F)C1]

To a solution of compound 55k (1.80 g, 6.03 mmol, 1.00 eq) in ACN (15.0 mL) was added NIS (2.31 g, 10.2 mmol, 1.7 eq) at 0° C. The mixture was stirred at 25° C. for 12 hrs. The reaction mixture was diluted with H₂O (20.0 mL) and extracted with DCM (10.0 mL×3). The combined organic layers were washed with brine (20.0 mL), dried over Na₂SO₄, filtered and concentrated under reduced pressure to give a residue. The residue was purified by column chromatography (SiO₂, Petroleum ether/Ethyl acetate=2/1, Rf=0.54) to give the compound 55l (2.40 g, 5.66 mmol, 93.7% yield) as a light yellow solid.

LCMS: Rt=0.471 min, m/z: 368.9, M−56+H⁺

### Step 13. Preparation of Intermediate 55m

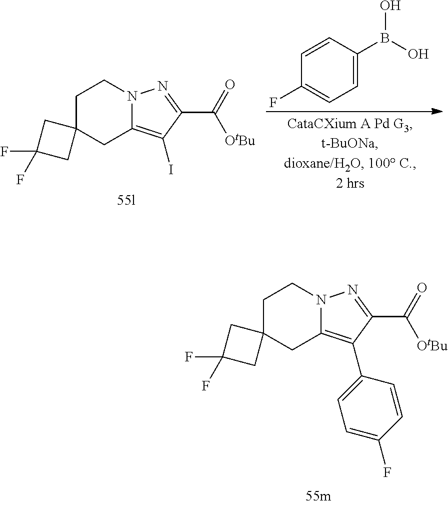 [SMILES: CC(C)(C)OC(=O)c1nn2c(c1I)CC1(CC2)CC(F)(F)C1 ; OB(O)c1ccc(F)cc1 ; CC(C)(C)OC(=O)c1nn2c(c1-c1ccc(F)cc1)CC1(CC2)CC(F)(F)C1]

To a solution of compound 55l (500 mg, 1.18 mmol, 1.00 eq) and compound (4-fluorophenyl)boronic acid (494 mg, 3.54 mmol, 3.00 eq) in H₂O (3.00 mL) and dioxane (5.00 mL) was added CataCxium A Pd G3 (85.8 mg, 117 μmol, 0.10 eq) and t-BuONa (226 mg, 2.36 mmol, 2.00 eq). The mixture was stirred at 100° C. for 2 hrs. The reaction mixture was diluted with H₂O (20.0 mL) and extracted with DCM (10.0 mL×3). The combined organic layers were washed with brine (20.0 mL), dried over Na₂SO₄, filtered and concentrated under reduced pressure to give a residue. The residue was purified by prep-TLC (SiO₂, Petroleum ether/Ethyl acetate=2/1, Rf=0.30) the compound 55m (452 mg, 1.08 mmol, 91.6% yield, 93.8% purity) as a light-yellow oil.

LCMS: m/z: 337.2, M−56+H⁺

### Step 14. Preparation of Intermediate 55n

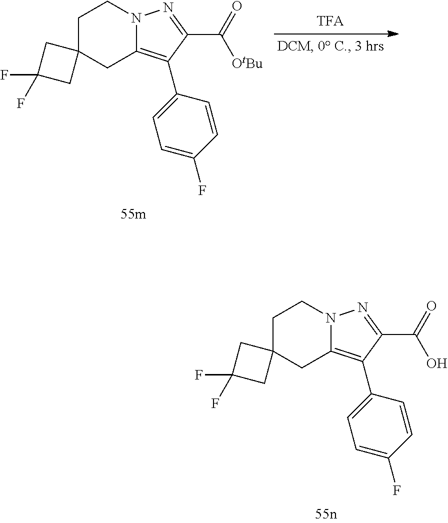 [SMILES: CC(C)(C)OC(=O)c1nn2c(c1-c1ccc(F)cc1)CC1(CC2)CC(F)(F)C1 ; O=C(O)c1nn2c(c1-c1ccc(F)cc1)CC1(CC2)CC(F)(F)C1]

To a solution of compound 55m (452 mg, 1.15 mmol, 1.00 eq) in DCM (5.00 mL) was added TFA (1.42 g, 12.4 mmol, 924 μL, 10.8 eq). The mixture was stirred at 0° C. for 2 hrs. The reaction mixture was diluted with H₂O (20.0 mL) and extracted with DCM (10.0 mL×3). The combined organic layers were washed with brine (20.0 mL), dried over Na₂SO₄, filtered and concentrated under reduced pressure to give the compound 55n (426 mg, 920 μmol, 79.9% yield, 97.3% purity, TFA) as a light yellow solid.

LCMS: m/z: 337.2, M+H⁺

### Step 15. Preparation of Intermediate 55o

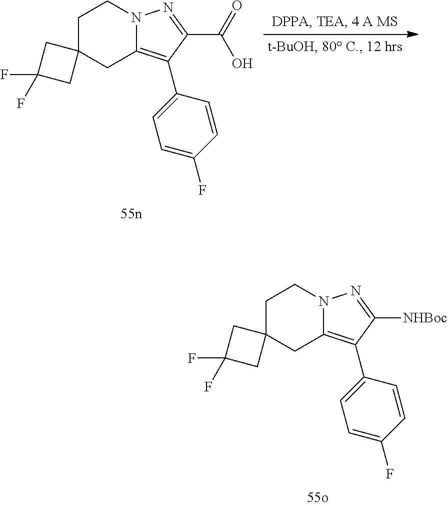 [SMILES: O=C(O)c1nn2c(c1-c1ccc(F)cc1)CC1(CC2)CC(F)(F)C1 ; CC(C)(C)OC(=O)Nc1nn2c(c1-c1ccc(F)cc1)CC1(CC2)CC(F)(F)C1]

To a solution of compound 55n (426 mg, 1.27 mmol, 1.00 eq) in t-BuOH (5.00 mL) was added DPPA (592 mg, 2.15 mmol, 464 μL, 1.70 eq) and TEA (448 mg, 4.43 mmol, 617 μL, 3.50 eq). The mixture was stirred at 80° C. for 12 hrs. The reaction mixture was diluted with H₂O (20.0 mL) and extracted with DCM (10.0 mL×3). The combined organic layers were washed with brine (20.0 mL), dried over Na₂SO₄, filtered and concentrated under reduced pressure to give a residue. The residue was purified by column chromatography (SiO₂, Petroleum ether/Ethyl acetate=0:1, Rf=0.68) to give the compound 550 (337 mg, 827 μmol, 65.3% yield) as a light yellow solid.

LCMS: m/z: 408.2, M+H⁺

### Step 16. Preparation of Intermediate 55p

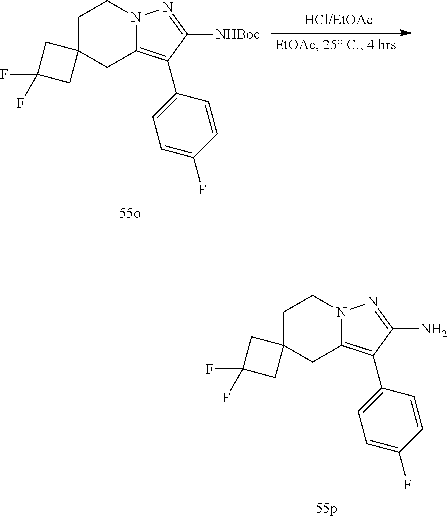 [SMILES: CC(C)(C)OC(=O)Nc1nn2c(c1-c1ccc(F)cc1)CC1(CC2)CC(F)(F)C1 ; Nc1nn2c(c1-c1ccc(F)cc1)CC1(CC2)CC(F)(F)C1]

To a solution of compound 550 (337 mg, 827 μmol, 1.00 eq) in EtOAc (1.00 mL) was added HCl/EtOAc (2 M, 2.00 mL, 4.84 eq). The mixture was stirred at 25° C. for 2 hrs. The reaction mixture was concentrated under reduced pressure to give the compound 55p (284 mg, 826 μmol, 99.8% yield, HCl) as a light yellow solid.

LCMS: m/z: 308.1 M+H⁺

### Step 17. Preparation of Intermediate 56

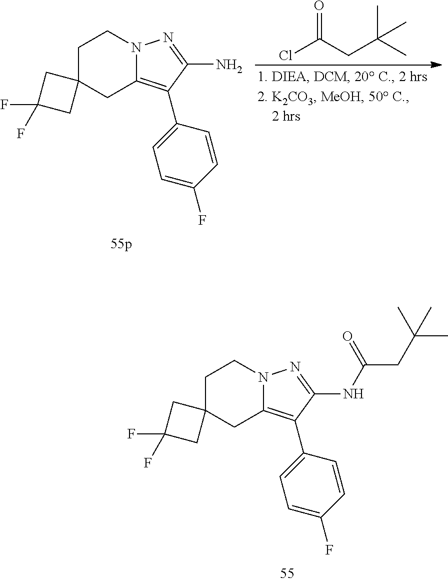

To a solution of compound 55p (284 mg, 924 μmol, 1.00 eq) in DCM (3.00 mL) was added DIEA (238 mg, 1.85 mmol, 321 μL, 2.00 eq) and compound 3,3-dimethylbutanoyl chloride (186 mg, 1.39 mmol, 192 μL, 1.50 eq). The mixture was stirred at 25° C. for 2 hrs. The reaction mixture was quenched by addition H₂O (20.0 mL), and then extracted with EtOAc (20.0 mL×2). The combined organic layers were washed with brine (20.0 mL), dried over Na₂SO₄, filtered and concentrated under reduced pressure to give a residue. To a solution of the residue (465 mg, 923 μmol, 1.00 eq) in MeOH (5.00 mL) was added K2CO3 (127 mg, 923 μmol, 1.00 eq). The mixture was stirred at 50° C. for 2 hrs. The residue was purified by prep-HPLC (neutral condition; column: CD24-WePure Biotech XPT C18 150×25×7 μm; mobile phase: [H₂O (10 mM NH₄OAc)-ACN]; gradient:37%-67% B over 11.0 min) to give compound 55 (78.0 mg, 228 μmol, 71.2% yield, 99.9% purity) as a white solid.

LCMS: Rt=2.091 min, m/z: 406.1, M+H⁺

1H NMR: 400 MHz, DMSO-d6 δ 9.31 (s, 1H), 7.31-7.34 (m, 2H), 7.15-7.19 (m, 2H), 4.07-4.10 (m, 2H), 2.92 (s, 2H), 2.60-2.66 (m, 2H), 2.44-2.49 (m, 2H), 2.13-2.14 (m, 2H), 2.06 (s, 2H), 0.93 (s, 9H).

### Example 7: Synthesis of Compound 56

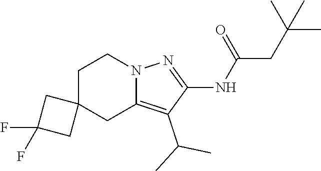 [SMILES: CC(C)c1c(NC(=O)CC(C)(C)C)nn2c1CC1(CC2)CC(F)(F)C1]

### Step 1. Preparation of Intermediate 56a

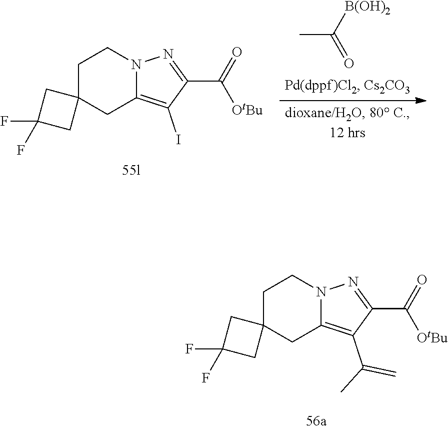

To a mixture of 55l (500 mg, 1.18 mmol, 1.00 eq), prop-1-en-2-ylboronic acid (396 mg, 2.36 mmol, 2.00 eq) and Cs₂CO₃ (1.15 g, 3.54 mmol, 3.00 eq) in dioxane (8 mL) and H₂O (2 mL) was degassed and purged with N2 3 times. Pd(dppf)Cl₂ (86.2 mg, 117 μmol, 0.10 eq) was added, and the mixture was stirred at 80° C. for 12 hours under N₂ atmosphere. The residue was diluted with ethyl acetate (10 mL) and water (10 mL). The aqueous phase was extracted with ethyl acetate (10 mL×3). The combined organic layers were dried over anhydrous sodium sulfate, filtered and concentrated under reduced pressure to afford a residue. The residue was purified by column chromatography (SiO₂, Commercial hexanes:Ethyl acetate=100/1 to 1/1) to give compound 56a (390 mg, 1.15 mmol, 97.7% yield) as a yellow solid.

LCMS: Rt=0.604 min, m/z=283.0, M−56+H⁺

1H NMR: (400 MHz, DMSO-d6) δ 5.15-5.10 (m, 1H), 4.76 (d, J=1.2 Hz, 1H), 4.15 (t, J=6.4 Hz, 2H), 3.32 (s, 3H), 2.81 (s, 2H), 2.66-2.55 (m, 2H), 2.47-2.35 (m, 2H), 2.14-2.08 (m, 2H), 1.47 (s, 9H)

### Step 2. Preparation of Intermediate 56b

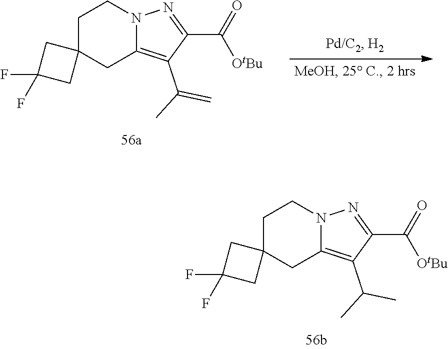 [SMILES: C=C(C)c1c(C(=O)OC(C)(C)C)nn2c1CC1(CC2)CC(F)(F)C1 ; CC(C)c1c(C(=O)OC(C)(C)C)nn2c1CC1(CC2)CC(F)(F)C1]

To a solution of 56a (390 mg, 1.15 mmol, 1.00 eq) in MeOH (10 mL) was added Pd/C (40.0 mg, 37.5 μmol, 10% purity), the mixture was stirred at 25° C. for 2 hours under H₂ (40 psi). The mixture was filtered with MeOH and concentrated under reduced pressure to afford a residue. The residue was purified by column chromatography (SiO₂, Commercial hexanes:Ethyl acetate=100/1 to 1/1) to give compound 56b (340 mg, 998 μmol, 86.6% yield) as a yellow oil.

LCMS: Rt=2.820 min, m/z=341.2 M+H⁺

1H NMR: (400 MHz, DMSO-d6) δ 4.15-4.09 (m, 2H), 3.40-3.33 (m, 1H), 3.33-3.31 (m, 2H), 2.66-2.55 (m, 2H), 2.48-2.37 (m, 2H), 2.08 (t, J=6.0 Hz, 2H), 1.49 (s, 9H), 1.18 (d, J=7.2 Hz, 6H)

### Step 3. Preparation of Intermediate 56c

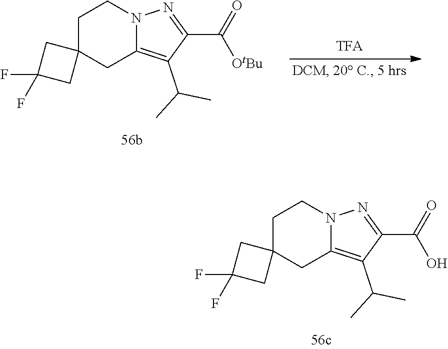 [SMILES: CC(C)c1c(C(=O)OC(C)(C)C)nn2c1CC1(CC2)CC(F)(F)C1 ; CC(C)c1c(C(=O)O)nn2c1CC1(CC2)CC(F)(F)C1]

To a solution of 56b (340 mg, 998 μmol, 1.00 eq) in DCM (5.00 mL) was added TFA (1.23 g, 10.7 mmol, 801 μL), and the mixture was stirred at 20° C. for 6 hours. The reaction mixture was concentrated under reduced pressure to afford a residue. The residue was purified by column chromatography (SiO₂, DCM:MeOH=10:1) to give compound 56c (230 mg, 809 μmol, 81.0% yield) as an off-white solid.

LCMS: Rt=0.458 min, m/z=285.1 M+H⁺

### Step 4. Preparation of Intermediate 56d

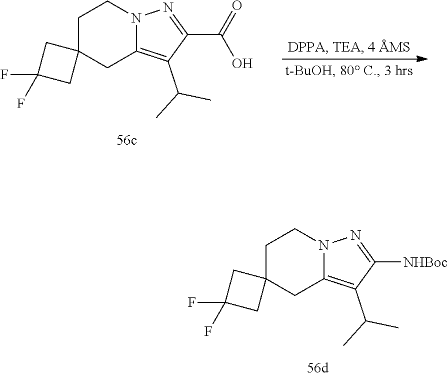 [SMILES: CC(C)c1c(C(=O)O)nn2c1CC1(CC2)CC(F)(F)C1 ; CC(C)c1c(NC(=O)OC(C)(C)C)nn2c1CC1(CC2)CC(F)(F)C1]

To a solution of 56c (200 mg, 703 μmol, 1.00 eq) and TEA (249 mg, 2.46 mmol, 342 μL, 3.50 eq) in t-BuOH (4.00 mL) was added DPPA (329 mg, 1.20 mmol, 258 μL, 1.70 eq) dropwise, the mixture was stirred at 80° C. for 3 hours under N₂. The reaction mixture was diluted with H2O (10 mL) and extracted with ethyl acetate (10 mL×3). The combined organic layers were washed with brine (5.0 mL), dried over Na₂SO₄, filtered and concentrated under reduced pressure to afford a residue. The residue was purified by column chromatography (SiO₂, Commercial hexanes:EtOAc=0/1) to give compound 56d (170 mg, 478.3 μmol, 67.9% yield) as a white solid.

LCMS: Rt=0.559 min, m/z=356.2 M+H⁺

1H NMR: (400 MHz, DMSO-d6)

δ 8.34 (s, 1H), 3.96 (t, J=6.0 Hz, 2H), 2.85 (s, 2H), 2.69-2.56 (m, 3H), 2.45-2.36 (m, 2H), 2.09-2.02 (m, 2H), 1.40 (s, 9H), 1.11 (d, J=7.2 Hz, 6H)

### Step 5. Preparation of Intermediate 56e

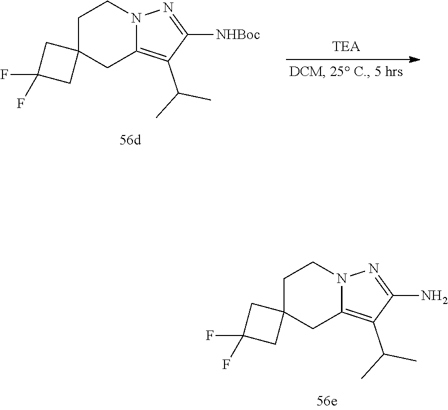 [SMILES: CC(C)c1c(NC(=O)OC(C)(C)C)nn2c1CC1(CC2)CC(F)(F)C1 ; CC(C)c1c(N)nn2c1CC1(CC2)CC(F)(F)C1]

To a solution of 56d (170 mg, 478 μmol, 1.00 eq) in DCM (2 mL) was added TFA (767 mg, 6.73 mmol, 0.50 mL, 14.1 eq), the mixture was stirred at 20° C. for 5 hours. The reaction mixture was concentrated under reduced pressure to give compound 56e (100 mg, 391 μmol, TFA salt, crude) as a yellow oil.

LCMS: Rt=0.353 min, m/z=256.1 M+H⁺

### Step 6. Preparation of Intermediate 56

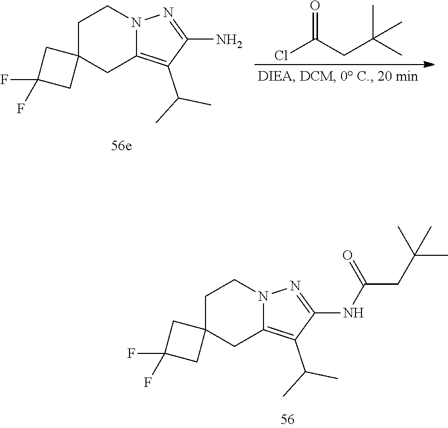

To a solution of 56e (100 mg, 391 μmol, 1.00 eq) in DCM (2 mL) was added DIEA (101 mg, 783 μmol, 136 μL, 2.00 eq) and 3,3-dimethylbutanoyl chloride (63.2 mg, 470 μmol, 65.2 μL, 1.20 eq) at 0° C. The mixture was stirred at 0° C. for 20 min. The reaction mixture was quenched with H₂O (10.0 mL) and then extracted with ethyl acetate (5 mL×3). The combined organic layers were washed with brine (10 mL), dried over Na₂SO₄, filtered and concentrated under reduced pressure. The residue was purified by preparative HPLC (column: CD24-WePure Biotech XPT C18 150*25*7 μm; mobile phase: [H₂O (10 mM NH₄HCO₃)-MeCN]; gradient: 35%-65% B over 12.0 min) to give compound 56 (62.0 mg, 175 μmol, 44.7% yield, 99% purity) as a white solid.

LCMS: m/z: 354.6, M+H⁺

HPLC: Rt=2.310 min

1H NMR: (400 MHz, DMSO-d6) δ 9.11 (s, 1H), 3.98 (s, 2H), 2.88 (s, 2H), 2.70-2.57 (m, 3H), 2.46-2.37 (m, 2H), 2.11 (s, 2H), 2.08-2.02 (m, 2H), 1.11 (d, J=7.1 Hz, 6H), 1.01 (s, 9H).

### Example 8: Synthesis of Compound 57

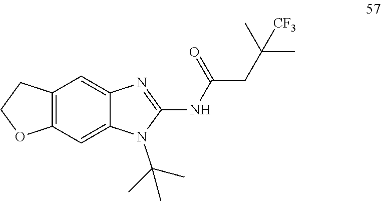 [SMILES: CC(C)(C)n1c(NC(=O)CC(C)(C)C(F)(F)F)nc2cc3c(cc21)OCC3]

### Step 1. Preparation of Intermediate 57a

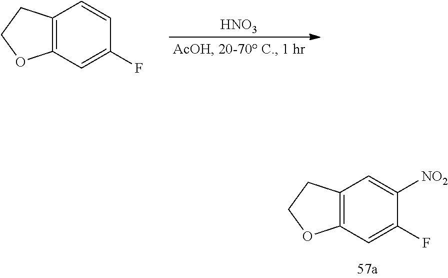 [SMILES: Fc1ccc2c(c1)OCC2 ; "HNO3" ; O=[N+]([O-])c1cc2c(cc1F)OCC2]

To a solution of compound 6-fluoro-2,3-dihydrobenzofuran (900 mg, 6.52 mmol, 1.00 eq) in AcOH (1.00 mL) was added HNO3 (492 mg, 7.82 mmol, 351 μL, 1.20 eq) at 20° C. The mixture was stirred at 70° C. for 1 hrs. The residue was diluted with ice water (30.0 mL) and extracted with EtOAc (30.0 mL×3). The combined organic layers were washed brine (30.0 mL×3), and dried over Na₂SO₄, filtered and concentrated under reduced pressure. The residue was purified by prep-TLC (SiO₂, Petroleum ether:EtOAc=2:1, Rf=0.60) to give compound 57a (390 mg, 2.13 mmol, 32.7% yield, 100% purity) as a yellow solid.

LCMS: Rt=0.356 min, m/z: 184.2, M+H⁺

### Step 2. Preparation of Intermediate 57b

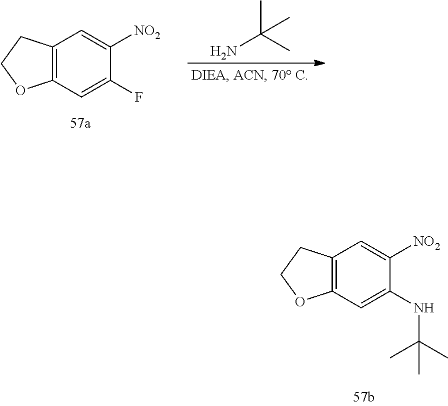

To a solution of compound 57a (390 mg, 2.13 mmol, 1.00 eq) and 2-methylpropan-2-amine (311 mg, 4.26 mmol, 447 μL, 2.00 eq) in ACN (5.00 mL) was added DIEA (550 mg, 4.26 mmol, 741 μL, 2.00 eq). The mixture was stirred at 70° C. for 12 hrs. The reaction mixture was quenched by addition H₂O 2.00 mL at 0° C. and then diluted with EtOAc and extracted with EtOAc (2.00 mL×3). The combined organic layers were dried over Na₂SO₄, filtered and concentrated under reduced pressure to give compound 57b (530 mg, crude) as a yellow solid.

LCMS: Rt=0.464 min, m/z: 259.2, M+Na⁺

### Step 3. Preparation of Intermediate 57c

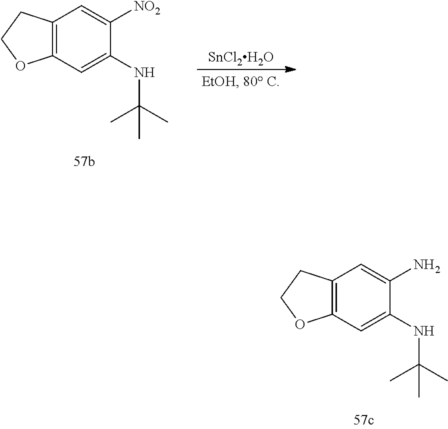 [SMILES: CC(C)(C)Nc1cc2c(cc1[N+](=O)[O-])CCO2 ; "SnCl2·H2O" ; CC(C)(C)Nc1cc2c(cc1N)CCO2]

To a solution of compound 57b (530 mg, 2.24 mmol, 1.00 eq) in EtOH (5.00 mL) was added carbononitridic bromide (2.02 g, 8.97 mmol, 4.00 eq). The mixture was stirred at 80° C. for 2 hrs. The reaction mixture was diluted with NaHCO₃ 3.00 mL and extracted with EtOAc (3.00 mL×3). The combined organic layers were washed with brine (3.00 mL×2), dried over Na₂SO₄, filtered and concentrated under reduced pressure to give compound 57c (430 mg, 2.08 mmol, 92.9% yield) as a yellow solid.

LCMS: Rt=0.215 min, m/z: 151.3, M−56+H⁺

### Step 4. Preparation of Intermediate 57d

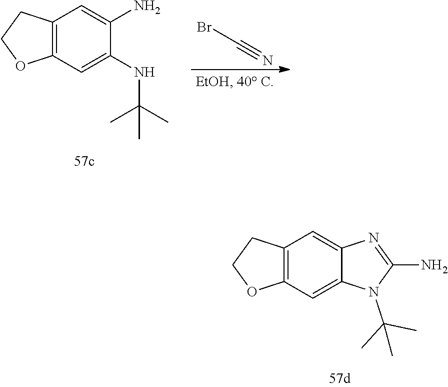

To a solution of compound 57c (420 mg, 2.04 mmol, 1.00 eq) in EtOH (2.00 mL) was added carbononitridic bromide (431 mg, 4.07 mmol, 298 μL, 2.00 eq). The mixture was stirred at 40° C. for 16 hrs. The reaction mixture was diluted with NaHCO₃ 3.00 mL and extracted with EtOAc (3.00 mL×3). The combined organic layers were washed with brine (3.00 mL×2), dried over Na₂SO₄, filtered and concentrated under reduced pressure to give compound 57d (493 mg, 2.02 mmol, 99.1% yield, 94.7% purity) as a yellow solid.

LCMS: Rt=0.259 min, m/z: 232.3, M+H⁺

### Step 5. Preparation of Compound 57

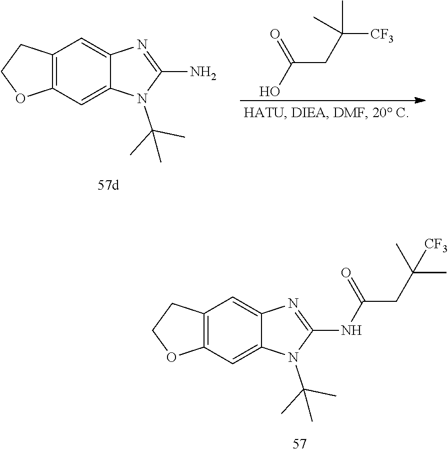 [SMILES: CC(C)(C)n1c(N)nc2cc3c(cc21)OCC3 ; CC(C)(CC(=O)O)C(F)(F)F ; CC(C)(C)n1c(NC(=O)CC(C)(C)C(F)(F)F)nc2cc3c(cc21)OCC3]

To a solution of compound 57d (150 mg, 648 μmol, 1.00 eq) and compound 4,4,4-trifluoro-3,3-dimethylbutanoic acid (132 mg, 778 μmol, 1.20 eq) in DMF (1.00 mL) was added HATU (295 mg, 778 μmol, 1.20 eq) and DIEA (251 mg, 1.95 mmol, 338 μL, 3.00 eq). The mixture was stirred at 25° C. for 2 hrs. The reaction mixture was diluted with H₂O 3.00 mL and extracted with EtOAc (3.00 mL×3). The combined organic layers were washed with brine (3.00 mL×2), dried over Na₂SO₄, filtered and concentrated under reduced pressure to give a residue. The residue was purified by prep-HPLC (neutral condition; column: Waters xbridge 150*25 mm 10 μm; mobile phase: [water (NH₄HCO₃)-ACN]; gradient:53%-83% B over 10 min) to give compound 57 (155 mg, 404 μmol, 62.3% yield, 100% purity) as a white solid.

LCMS: Rt=0.384 min, m/z: 384.3, M+H⁺

HPLC: Rt=2.385 min

¹H NMR: 400 MHz, CDCl₃ δ12.9 (s, 1H), 7.14 (s, 1H), 7.03 (s, 1H), 4.60 (t, j=8.8 Hz, 2H), 3.26 (t, j=8.8 Hz, 2H), 2.59 (s, 2H), 1.89 (s, 9H), 1.26 (s, 6H).

The preparation of the compounds listed below refers to Example 8

| Name | Structure | Analytic Data |
|---|---|---|
| Compound 58 | 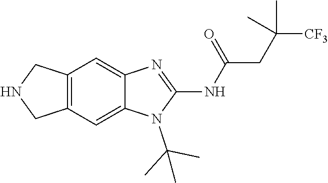 [SMILES: CC(C)(C)n1c(NC(=O)CC(C)(C)C(F)(F)F)nc2cc3c(cc21)CNC3] | ¹H NMR 400 MHz, CDCl3 δ 8.46 (s, 1H), 7.60 (s, 1H), 7.15 (s, 1H), 4.56 (d, J = 8.4 Hz, 4H), 2.63 (s, 2H), 1.93 (s, 9H), 1.33 (s, 6H). MS m/z (ESI): 383.3, M + H⁺ Purity by HPLC: 94.8% |
| Compound 59 | 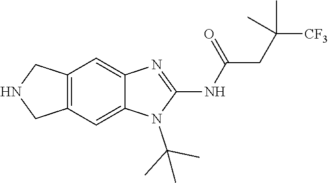 | ¹H NMR 400 MHz, DMSO-d₆ δ 13.1 (s, 1H), 7.70 (d, j = 22.4 Hz, 2H), 6.70 (s, 1H), 4.51 (s, 2H), 2.65 (s, 2H), 1.97 (s, 9H), 1.27 (s, 6H). MS m/z (ESI): 397.1, M + H⁺ Purity by HPLC: 99.8% |

### Example 9: Synthesis of Compound 60

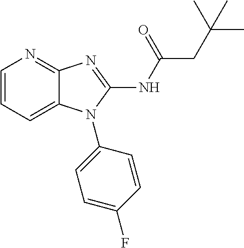 [SMILES: CC(C)(C)CC(=O)Nc1nc2ncccc2n1-c1ccc(F)cc1]

### Step 1. Preparation of Intermediate 60a

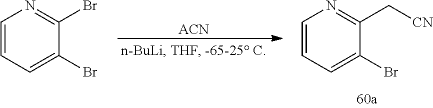 [SMILES: Brc1cccnc1Br ; "ACN" ; N#CCc1ncccc1Br]

To a solution of acetonitrile (1.30 g, 31.6 mmol, 1.67 mL, 1.50 eq) in THF (50.0 mL) was added dropwise n-BuLi (2.5 M, 12.6 mL, 1.50 eq) at −65° C. After addition, the mixture was stirred at −65° C. for 1 hrs, and then compound 2,3-dibromopyridine (5.00 g, 21.1 mmol, 1.00 eq) was added dropwise at −65° C. The resulting mixture was stirred at 25° C. for 11 hrs. The reaction mixture was diluted with aqueous NH₄Cl (80.0 mL) and extracted with EtOAc(100 mL×2). The combined organic layers were washed with brine (100 mL), dried over Na₂SO₄, filtered and concentrated under reduced pressure to give a residue. The residue was purified by column chromatography (SiO₂, Petroleum ether/Ethyl acetate=2:1, R_{f}=0.37) to give compound 60a (1.01 g, 5.13 mmol, 24.3% yield) as a white solid.

LCMS: Rt=0.251 min, m/z: 197.1, M+H⁺

### Step 2. Preparation of Intermediate 60b

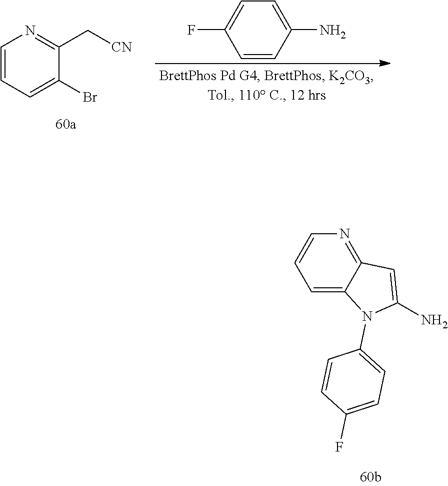 [SMILES: N#CCc1ncccc1Br ; Nc1ccc(F)cc1 ; Nc1cc2ncccc2n1-c1ccc(F)cc1]

To a solution of compound 60a (400 mg, 2.03 mmol, 1.00 eq) and 4-fluoroaniline (270 mg, 2.44 mmol, 233 μL, 1.20 eq) in Tol. (1.00 mL) was added K₂CO₃ (561 mg, 4.06 mmol, 2.00 eq) and Brettphos (108 mg, 203 μmol, 0.10 eq) and BrettPhos (Pd, G4) (186 mg, 203 μmol, 0.10 eq). The mixture was stirred at 110° C. for 12 hrs. The reaction mixture was quenched by addition H₂O (30.0 mL) at 0° C. and then extracted with EtOAc (20.0 mL*3). The combined organic layers were washed with brine (20.0 mL*2), dried over Na₂SO₄, filtered and concentrated under reduced pressure to give a residue. The residue was purified by column chromatography (SiO₂, Petroleum ether/Ethyl acetate=0:1, R_{f}=0.10) to give compound 60b (100 mg, 440 μmol, 21.7% yield) as a yellow solid.

LCMS: Rt=1.144 min, m/z: 228.1, M+H⁺

### Step 3. Preparation of Compound 60

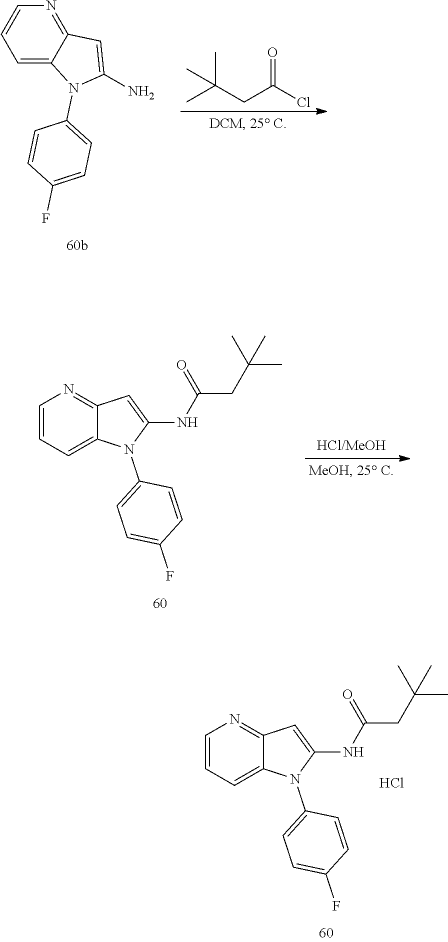

To a solution of compound 60b (90.0 mg, 396 μmol, 1.00 eq) in DCM (3.00 mL) was added DIEA (51.1 mg, 396 μmol, 68.9 μL, 1.00 eq) and 3,3-dimethylbutanoyl chloride (53.3 mg, 396 μmol, 55.0 μL, 1.00 eq). The mixture was stirred at 25° C. for 2 hrs. The reaction mixture was quenched by addition H₂O 10.0 mL at 0° C. and then extracted with EtOAc (10.0 mL*3). The combined organic layers were washed with brine (10.0 mL*2), dried over Na₂SO₄, filtered and concentrated under reduced pressure to give a residue. The residue was purified by column chromatography (SiO₂, Petroleum ether/Ethyl acetate=2:1, R_{f}=0.35) to give compound 60 (95.0 mg, 291 μmol, 73.7% yield) as a yellow solid.

To a solution of compound 60 (90.0 mg, 276 μmol, 1.00 eq) in MeOH (2.00 mL) was added HCl/MeOH (2 M, 2.00 mL). The mixture was stirred at 25° C. for 2 hrs. The reaction mixture was concentrated under reduced pressure to give 60 (80.0 mg, 243 μmol, 88.2% yield, 99.2% purity) as a yellow solid.

LCMS: Rt=0.327 min, m/z: 326.2, M+H⁺

HPLC: 10-80AB_6 min, Rt=1.680 min

¹H NMR: 400 MHz, DMSO-d₆ δ10.26 (s, 1H), 8.54 (d, J=5.2 Hz, 1H), 7.92 (d, J=8.0 Hz, 1H), 7.61-7.65 (m, 2H), 7.50-7.55 (m, 2H), 7.45-7.46 (m, 1H), 7.20 (s, 1H), 2.32 (s, 2H), 0.97 (s, 9H).

The preparation of the compounds listed below refers to Example 9

| Name | Structure | Data |
|---|---|---|
| Compound 61 | 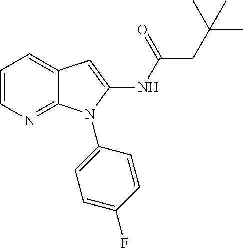 [SMILES: CC(C)(C)CC(=O)Nc1cc2cccnc2n1-c1ccc(F)cc1] | MS m/z (ESI): 326.3, M + H⁺ |

### Example 10: Synthesis of Compound 62

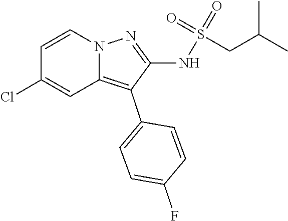 [SMILES: CC(C)CS(=O)(=O)Nc1nn2ccc(Cl)cc2c1-c1ccc(F)cc1]

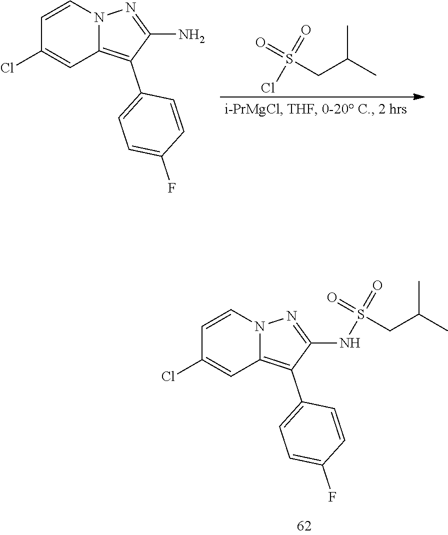

To a solution of 5-chloro-3-(4-fluorophenyl)pyrazolo[1,5-a]pyridin-2-amine (150 mg, 573 μmol, 1.00 eq) in THF (1.00 mL) was added dropwise i-PrMgCl (2 M, 573 μL, 2.00 eq) at 0° C. After addition, the mixture was stirred at 0° C. for 0.5 hr, and then 2-methylpropane-1-sulfonyl chloride (897 mg, 5.73 mmol, 10.0 eq) was added dropwise at 0° C. The resulting mixture was stirred at 20° C. for 2 hrs. The reaction mixture was quenched by addition H₂O (20.0 mL) and then extracted with EtOAc (20.0 mL×2). The combined organic layers were washed with brine (20.0 mL), dried over Na₂SO₄, filtered and concentrated under reduced pressure to give a residue. The residue was purified by prep-HPLC (neutral condition; column: CD 02-Waters Xbridge BEH C18 150*25*10 μm; mobile phase: [water (NH₄HCO₃)-ACN]; gradient: 42%-72% B over 10 min) give 62 (20.0 mg, 48.5 μmol, 8.47% yield, 92.7% purity).

LCMS: Rt=0.477 min, m/z: 382.1, Rt=0.503 min, m/z: 416.0, M+H⁺

¹H NMR: 400 MHz, DMSO-d₆ δ 9.99 (s, 1H), 8.72 (d, J=7.2 Hz, 1H), 7.62-7.69 (m, 3H), 7.30-7.35 (m, 2H), 7.00-7.02 (m, 1H), 3.33 (s, 2H), 2.22-2.24 (m, 1H), 1.06 (s, 6H).

The preparation of the compounds listed below refers to Example 10

| Name | Structure | Data |
|---|---|---|
| Compound 63 | 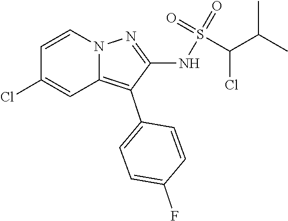 [SMILES: CC(C)C(Cl)S(=O)(=O)Nc1nn2ccc(Cl)cc2c1-c1ccc(F)cc1] | ¹H NMR 400 MHz, DMSO-d₆ δ δ 10.1 (s, 1H), 8.76 (d, J = 7.6 Hz, 1H), 7.73 (s, 1H), 7.71-7.72 (m, 2H), 7.30-7.35 (m, 2H), 7.04-7.07 (m, 1H), 5.55 (d, J = 1.6 Hz, 1H), 2.73- 2.75 (m, 1H), 1.06 (s, 6H) MS m/z (ESI): 416.1, M + H⁺ |

### BIOLOGICAL TEST EVALUATION

### Test Example 1: KNCQ2/3 Activation Assay

The ability of compounds to potentiate K-currents in HEK293 cells with overexpressed KNCQ2/3 (Kv7.2/7.3) was assessed using patch clamp on the QPatch automated screening platform.

Cell Line: The HEK293 cell line with overexpressed Kv7.2/7.3 was obtained from ICE Bioscience Inc. These HEK cells have been engineered to overexpress the Kv7.2/3 ion channels.

Cell Culture: Cells were maintained in the media containing DMEM (Corning, cat. #10-013-CV), 10% FBS (Gibco, cat. #10099141C), 800 μg/mL G418 (GPC, cat. #AK108) and 200 μg/mL hygromycin B (Solarbio, cat. #H8080). The cells were incubated at 37° C. in a humidified incubator under 5% carbon dioxide. Cells were grown in 6 cm dishes to ˜80% confluency. Currents were recorded 24 hours after channel induction.

Compound Plates: Test compounds were prepared by performing serial dilutions in DMSO (Sigma cat #D4540). Final dilutions were made in external recording solution with a final DMSO concentration of 0.1% DMSO. For single concentration screens each plate had 10 μM retigabine (Selleck Chemicals, cat. #S4733) as a positive control.

Electrophysiology: External solution contained 140 mM NaCl (Sigma cat. #S5886), 3.5 mM KCl (Sigma cat. #P5405), 1 mM MgCl₂·6H₂O (Sigma cat. #M2393), 2 mM CaCl₂·2H₂O (Sigma cat. #C7902), 10 mM D-Glucose (Sigma cat. #G8270), 10 mM HEPES (Santa Cruz cat. #SC-29097A), 1.25 mM NaH₂PO₄·2H₂O, pH was adjusted to 7.4 with NaOH. Internal solution contained 125 mM K-Aspartic (Sigma cat. #G4500), 20 mM KCl, 10 mM EGTA (Sigma cat. #E3889), 1 mM MgCl₂·6H₂O, 5 mM Mg-ATP (Sigma cat. #A9187), 5 mM HEPES, pH was adjusted to 7.2 with KOH.

On the day of the experiment cells are washed with the external solution and harvested with 0.25% Trypsin EDTA (Gibco cat. #25200-072). Cells were then centrifuged at 1000 rpm/min for 5 minutes and resuspended in the external solution at ˜2.5×10⁶ cells/mL. The cells were stirred with an equilibrium sharker for 20 minutes at room temperature before experiments started.

Potassium channel activity was measured on the QPatch 48X (Sophion Bioscience) using QPlates with 48-wells plate. The KCNQ current was recorded when the voltage was held at −80 mV for 3s, then stepped to −30 mV for 500 ms and back to 80 mV. The process was repeated with an increment of 10 mV between −80 mV and +40 mV. Each cell was taken as an independent experiment and only one compound was tested per well.

Single Concentration Screen: Baseline conditions were obtained by recording 5 sweeps in the external solution only, this was repeated for three applications of the external solution. The effect of test compounds on elicited current was then assessed by recording 5 sweeps in the presence of a 1 μM compound solution. The steady-state current at the end of the 2 s pulse to +40 mV was measured to determine the fold increase from baseline.

Data of the Kv7.2/7.3 Activation Assay is summarized in Table below

TABLE 1
Kv7.2/7.3 QPatch Single Concentration Screen Results

| Name | Kv7.2/7.3 Activity (1 μM) |
|---|---|
| 1 | + |
| 3 | + |
| 7 | + |
| 8 | ++ |
| 10 | +++ |
| 11 | ++ |
| 12 | ++ |
| 14 | ++ |
| 15 | +++ |
| 16 | + |
| 17 | ++ |
| 20 | +++ |
| 32 | +++ |
| 33 | +++ |
| 34 | ++ |
| 35 | +++ |
| 36 | +++ |
| 37 | + |
| 43 | ++ |
| 44 | + |
| 47 | + |
| 53 | + |
| 54 | ++ |
| 55 | +++ |
| 60 | + |
| 64 | + |
| 65 | ++ |

*Increase in current from HEK293 cells with overexpressed Kv7.2/3, measured at compounds concentration of 1 μM described as a range from ≤100% (−), 100%-199.99% (+), 200%-299.99% (++), and ≥300% (+++) fold increase over baseline.

### Test Example 2: Epilepsy Model

### Experimental Procedure

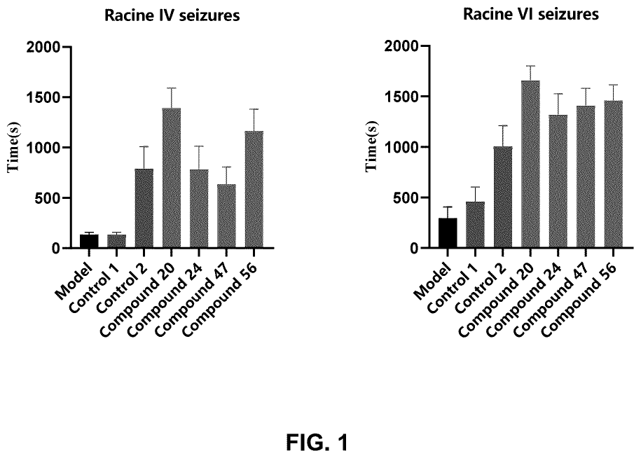

SPF-grade male ICR mice (22-24 g) were purchased from Human Silaike Jingda Laboratory Animal Co. Ltd., China and sent to the housing facility after veterinary inspection to adapt to the rearing environment. The mice were housed in cages for 3 days under suitable conditions (temperature at 22±1° C., humidity at 40-70%, sufficient water and food, 12 hours of daylight). On the formal test day, all mice were weighed, and those meeting the weight criteria were numbered at the base of their tails. Mice that did not meet the weight standard continued adapting until they reached the required weight, while overweight mice were excluded. Animals were orally administered compound 34 (10 mg/kg) 30 min before the i.p. injection of PTZ (100 mg/kg, Sigma, USA) for epilepsy modeling. The seizure response of the mice was evaluated based on the epilepsy grading criteria. The latency and incidence rate of each seizure grade were recorded for 30 min. After the experiment, the test animals were euthanized using carbon dioxide. The results are shown in Table 2 and FIG. 1 respectively.

### Experimental Results

TABLE 2

| | | Latency to Racine | Latency to Racine |
|---|---|---|---|
| | | IV Seizure | VI Seizure |
| Group | Name | (s) | (s) |
| Reference | Reference compound 1 | 134 | 460 |
| Reference | Reference compound 3 | 790 | 1005 |
| Test group 1 | 34 | 1393 | 1658 |
| Test group 2 | 38 | 784 | 1320 |
| Test group 3 | 62 | 636 | 1409 |
| Test group 4 | 75 | 1164 | 1458 |
| Model | Vehicle | 218 | 270 |

The experimental results indicate that in the PTZ mouse model, oral administration of the invented compound at 10 mg/kg exhibits significant activity, effectively prolonging seizure duration and reducing epilepsy-related mortality to some extent, outperforming the control compound.

### Test Example 3: In Vivo Activities in 2% Formalin Rat Model

SPF male SD rats (170-190 g) were purchased from Human Silaike Jingda Laboratory Animal Co. Ltd., China. The rats were housed in cages for 3 days under suitable conditions (temperature at 22±1° C., humidity at 40-70%, sufficient water and food, 12 hours of daylight). The rats with a body weight of 220-240 g were numbered for further experiments. The rats were placed in the ZH-PAN801 automated rodent behavioral tracking system (Anhui Zhenghua Biologic Apparatus Facilities) for 60 min per day for an additional 3 days.

Paw licking behavior, a correlation of spontaneous pain, was tracked using the ZH-PAN801 automated rodent behavioral tracking system that can differentiate behaviors and locomotor activity of individually housed rats. The ZH-PAN801 system consists of sensor platforms which rest on two orthogonally placed force transducers and a third fixed point attached to a base plate. Each force transducer transforms the mechanical vibrations caused by animal movement into electrical signals, which are amplified, filtered, and digitized by the ZH-PAN801 system. Characteristic movements have their own unique vibrational patterns which the ZH-PAN801 software can distinguish. The cylinder cages (75 mm (radius)×310 mm (height)) consist of two halves to sensitize detection of vibrations. Thus, the base is separate from the sides, the lid, and sits directly on the sensor platform, while the sides and the lid hang from supports on the base plate.

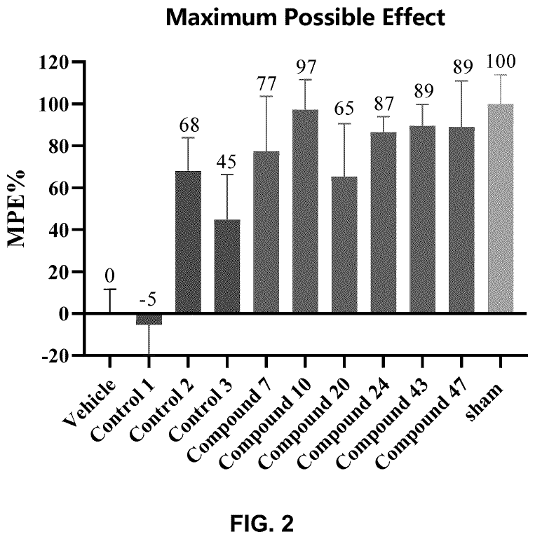

Rats were equipped with the force transducer 15 min before the dosing. Animals were orally administered compound 21 (10 mg/kg) 15 min before the formalin injection. Rats were administered an intraplantar injection of formalin (50 μL, 2.0%, Sigma, USA) into the dorsal surface of the hindpaw attached with the force transducer to induce acute pain and placed in the automated rodent behavioral tracking system. Paw licking behavior was monitored for 60 min using the ZH-PAN801 behavior tracking system. The duration of paw licking behavior (in minutes) was collected from two distinctive phases, the early nociceptive phase occurring 0-10 min after formalin injection and the late phase thought to be associated with central sensitization, which peaks 11-60 min after formalin injection. Treatment effects were compared to vehicles by an unpaired t-test and one-way ANOVA using Graph Pad 9.0. The results are shown in Table 3 and FIG. 2 respectively.

### Test Results:

TABLE 3

| | | % MPE |
|---|---|---|
| Group | Name | (10 mpk) |
| Reference | Reference compound 1 | −5% |
| Reference | Reference compound 2 | 68% |
| Reference | Reference compound 3 | 45% |
| Test group 1 | 21 | 77% |
| Test group 2 | 58 | 89% |
| Test group 3 | 24 | 97% |
| Test group 4 | 34 | 65% |
| Test group 5 | 38 | 87% |
| Test group 6 | 62 | 89% |

1. Some embodiments include a compound represented by Formula (I), or a stereoisomer, tautomer, pharmaceutically acceptable salt, solvate, deuterated derivative, metabolite, or prodrug, 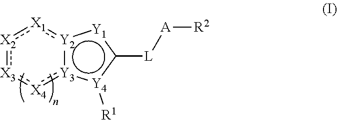
Wherein,
n is 0-5;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 cycloalkenyl, C3-C8 heterocycle, or Ar, wherein each occurrence of R² is optionally substituted with one or more substituents independently selected from the group consisting of hydroxyl (—OH), amino (—NH₂), cyano (—CN), halogen, C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
L is optionally selected from —NHCO— or —NHSO₂—;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR³—, —CR⁴R⁵—, —N—, —NR⁶—, —O—, or —S—;
wherein 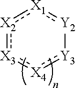
is selected from the group consisting of the following structures, 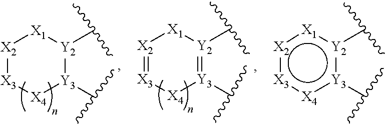
In an embodiment, each of R³, R⁴ and R⁵ is independently selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C2-C6 alkenyl, C3-C8 cycloalkyl, C1-C6 alkoxy, halogenated C1-C6 alkoxy, —C(═O)OR′, —C(═O)R′, —NHR′, or —NR′R″; wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from the group consisting of —NH₂ or C1-C3 alkyl,
R⁶ is selected from the group consisting of hydrogen (H), —(C═O)(C1-C6 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;
Any one or two Xs, together with the carbon atoms to which they are attached, form a ring optionally containing 1-3 heteroatoms selected independently from nitrogen (N), oxygen (O), and sulfur (S), including 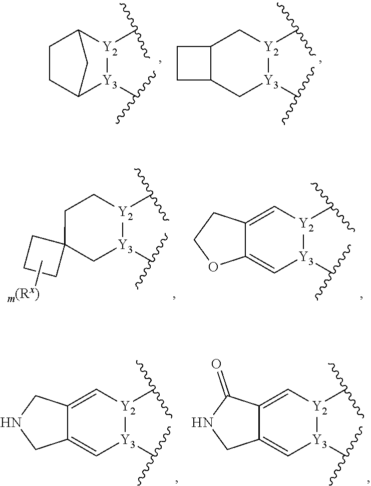
Wherein,
m is 0-6;
R^{x} is selected from the group consisting of hydrogen (H), halogen, cyano (—CN), C1-C6 alkyl;
Y₁, Y₂, Y₃, and Y₄ are each independently selected from the group consisting of C, N, O, or S;
More preferably, Y₁, Y₂, Y₃, and Y₄ are each independently selected from C or N, with examples including:
When Y₁ and Y₄ are N, Y₂ and Y₃ are C;
When Y₁ and Y₃ are N, Y₂ and Y₄ are C;
When Y₁ and Y₂ are N, Y₃ and Y₄ are C;
When Y₁, Y₂, and Y₃ are C, Y₄ is N.
Wherein, 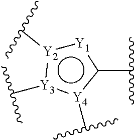
is selected from the group consisting of the following structure: 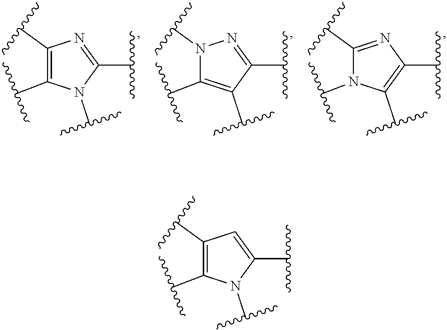
Wherein, when 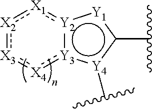 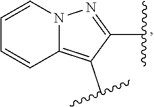 [SMILES: *c1nn2ccccc2c1*]
L is —NHSO₂—
Wherein, when 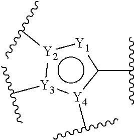
is 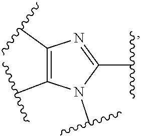 [SMILES: *c1nc(*)n(*)c1*] 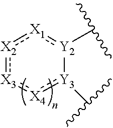
is selected from the group consisting of the following structure:

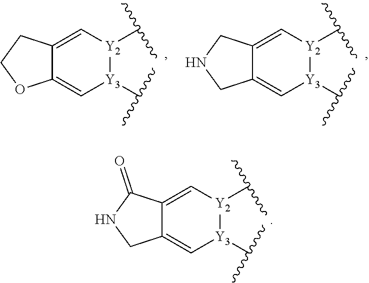

2. The compound of Formula (I) according to claim 1, or a stereoisomer, tautomer, pharmaceutically acceptable salt, solvate, deuterated analog, metabolite, or prodrug thereof, wherein the compound is a compound of Formula (I-1), or a stereoisomer, tautomer, pharmaceutically acceptable salt, solvate, deuterated analog, metabolite, or prodrug thereof; 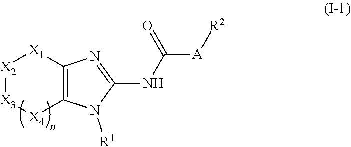
Wherein,
n is 0-5;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
In some preferred embodiments, R² is selected from the group consisting of C3-C8 heterocycle, or Ar, wherein each occurrence of R² is optionally substituted with one or more substituents independently selected from the group consisting of fluorine (—F), chlorine (—Cl), bromine (—Br), iodine (—I), C1-C6 alkyl, halogenated C1-C6 alkyl; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); 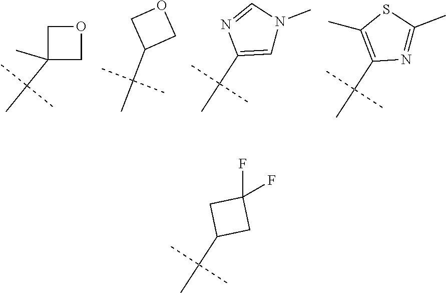
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;
In an embodiment, R⁴ and R⁵ are each independently selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″;
wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of H, —(C═O)(C1-C4 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;
In a preferred embodiment, X₁, X₂, X₃, and X₄ are each independently selected from CH₂.

3. The compound of Formula (I) according to claim 1, or a stereoisomer, tautomer, pharmaceutically acceptable salt, solvate, deuterated analog, metabolite, or prodrug thereof, wherein the compound is a compound of Formula (I-2), or a stereoisomer, tautomer, pharmaceutically acceptable salt, solvate, deuterated analog, metabolite, or prodrug thereof: 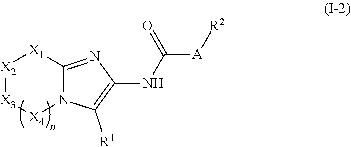
Wherein,
n is 0-5;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally containing 1-4 ring heteroatoms selected independently from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;
In an embodiment, R⁴ and R⁵ are each independently selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″;
wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of H, —(C═O)(C1-C4 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;
In a preferred embodiment, X₁, X₂, X₃, and X₄ are each independently selected from —CH₂—.

4. The compound of Formula (I) according to claim 1, or a stereoisomer, tautomer, pharmaceutically acceptable salt, solvate, deuterated analog, metabolite, or prodrug thereof, wherein the compound is a compound of Formula (I-3), or a stereoisomer, tautomer, pharmaceutically acceptable salt, solvate, deuterated analog, metabolite, or prodrug thereof: 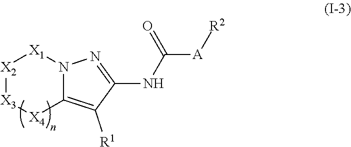
Wherein,
n is 0-5;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R2 is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR⁴R⁵—, —NR⁶—, —O—, or —S—;
In an embodiment, R⁴ and R⁵ are each independently selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″;
wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of H, —(C═O)(C1-C4 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;
Any one or two Xs, together with the carbon atoms to which they are attached, form a C3-C6 ring optionally containing 1-3 heteroatoms selected independently from nitrogen (N), oxygen (O), and sulfur (S), including 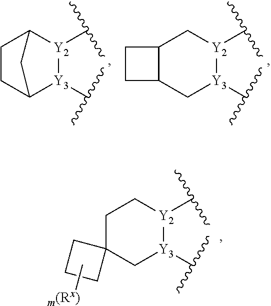
Wherein,
m is 0-6;
R^{x} is selected from the group consisting of hydrogen (H), halogen, cyano (—CN), C1-C6 alkyl;
Preferably, R⁴ and R⁵, together with the carbon atoms to which they are attached, may form a ring of the following formula, 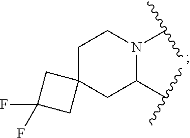 [SMILES: *C1CC2(CCN1*)CC(F)(F)C2]
or
any two Xs can be linked to form the following structure,

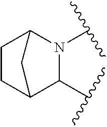 [SMILES: *C1C2CCC(C2)N1*]

5. The compound of Formula (I) according to claim 1, or a stereoisomer, tautomer, pharmaceutically acceptable salt, solvate, deuterated analog, metabolite, or prodrug thereof, wherein the compound is a compound of Formula (I-4), or a stereoisomer, tautomer, pharmaceutically acceptable salt, solvate, deuterated analog, metabolite, or prodrug thereof: 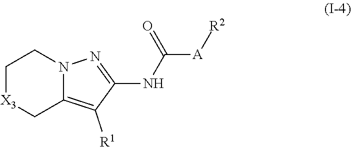
Wherein,
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl or C3-C8 heterocycle, wherein each occurrence of R² is optionally substituted with one or more substituents independently selected from the group consisting of fluorine (—F), chlorine (—Cl), bromine (—Br), iodine (—I), C1-C6 alkyl, halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₃ is —CR⁴R⁵—, —NR⁶—;
In an embodiment, R⁴ and R⁵ are each independently selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, each of which is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″;
wherein R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
R⁶ is selected from the group consisting of H, —(C═O)(C1-C4 alkyl), C2-C6 alkenyl, C1-C6 alkyl, or C3-C8 cycloalkyl, wherein each occurrence of R⁶ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R′ and R″ are each independently selected from the group consisting of C1-C6 alkyl or halogenated C1-C6 alkyl;
R⁴ and R⁵ are each independently selected from the group consisting of H, F, Cl, Br, I, OH, CN, —C(═O)OH, methyl, ethyl, propyl, isopropyl, butyl, tert-butyl, CH₂F, CHF₂, CF₃, methoxy, ethoxy, propoxy, butoxy, substituted C1-C6 alkyl, substituted C1-C6 alkoxy, each of which is optionally substituted with one or more substituents independently selected from the group consisting of fluorine (—F), chlorine (—Cl), bromine (—Br), iodine (—I);
R⁶ is selected from the group consisting of H, —(C═O)methyl, —(C═O)ethyl, —(C═O)propyl, vinyl, propenyl, allyl, methyl, ethyl, propyl, isopropyl, butyl, tert-butyl, cyclopropyl, cyclobutyl, cyclopentyl, cyclohexyl, cyclobutylmethyl, C2-C6 alkenyl, C1-C6 alkyl, or C3-C8 cycloalkyl, wherein each occurrence of R⁶ is optionally substituted with one or more substituents independently selected from the group consisting of halogen, methyl, ethyl, propyl, isopropyl;

6. The compound of Formula (I) according to claim 1, or a stereoisomer, tautomer, pharmaceutically acceptable salt, solvate, deuterated analog, metabolite, or prodrug thereof, wherein the compound is a compound of Formula (I-5), or a stereoisomer, tautomer, pharmaceutically acceptable salt, solvate, deuterated analog, metabolite, or prodrug thereof: 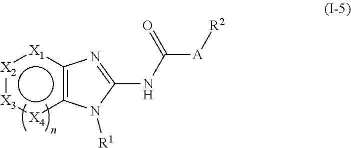
Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R2 is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH2), cyano (—CN), halogen, nitro (—NO2), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
Preferably, R¹ is C(CH₃)₃, R² is C(CH₃)₂CF₃, and A is CH₂, X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
Two adjacent R³ groups and the atoms to which they are attached together form C3-C8 heterocycloalkyl or C3-C8 heterocycloalkenyl selected from the group consisting of the following structures:

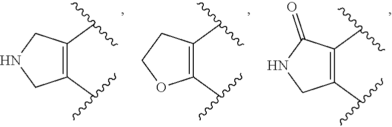 [SMILES: *C1=C(*)CNC1 ; *C1=C(*)OCC1 ; *C1=C(*)C(=O)NC1]

7. The compound of Formula (I) according to claim 1, or a stereoisomer, tautomer, pharmaceutically acceptable salt, solvate, deuterated analog, metabolite, or prodrug thereof, wherein the compound is a compound of Formula (I-6), or a stereoisomer, tautomer, pharmaceutically acceptable salt, solvate, deuterated analog, metabolite, or prodrug thereof: 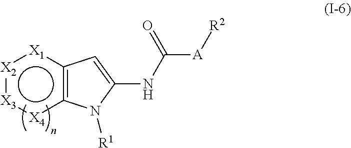
Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
A is C1-C6 alkyl, wherein A is optionally substituted with one or more substituents independently selected from the group consisting of hydroxyl (—OH), amino (—NH₂), cyano (—CN), halogen, nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; R′ and R″ are each independently selected from the group consisting of C1-C6 alkyl or halogenated C1-C6 alkyl;
X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;
In some preferred embodiments, X₁, X₂, X₃, and X₄ are each independently selected from —CH— or —N— to form the group consisting of the following structures:

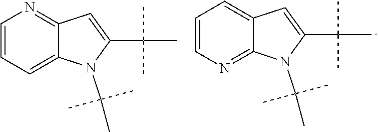

8. The compound of Formula (I) according to claim 1, or a stereoisomer, tautomer, pharmaceutically acceptable salt, solvate, deuterated analog, metabolite, or prodrug thereof, wherein the compound is a compound of Formula (I-7), or a stereoisomer, tautomer, pharmaceutically acceptable salt, solvate, deuterated analog, metabolite, or prodrug thereof: 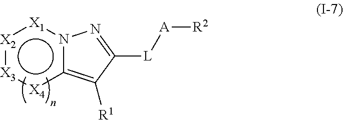
Wherein,
n is 0-3;
R¹ is selected from the group consisting of C1-C6 alkyl, C2-C6 alkenyl, C3-C8 cycloalkyl, Ar, C3-C8 cycloalkenyl, or C3-C8 heterocycle, wherein each occurrence of R¹ is optionally substituted with one or more substituents independently selected from the group consisting of hydroxyl (—OH), amino (—NH₂), cyano (—CN), fluorine (—F), chlorine (—Cl), bromine (—Br), iodine (—I), nitro (—NO₂), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
R² is selected from the group consisting of C1-C6 alkyl, C2-C6 alkenyl, C3-C8 cycloalkyl, C3-C8 heterocycle, or Ar, wherein each occurrence of R² is optionally substituted with one or more substituents independently selected from the group consisting of hydroxyl (—OH), amino (—NH₂), cyano (—CN), fluorine (—F), chlorine (—Cl), bromine (—Br), iodine (—I), C1-C6 alkyl, halogenated C1-C6 alkyl, C1-C6 alkoxy, halogenated C1-C6 alkoxy, C3-C8 cycloalkyl, halogenated C3-C8 cycloalkyl, —C(═O)OR′, —C(═O)R′, —NHR′, or —NR′R″; Ar is a 5- to 10-member mono- or bicyclic aromatic group, optionally comprising 1 to 4 heteroatoms as part of the ring structure, wherein said heteroatoms are independently selected from nitrogen (N), oxygen (O), and sulfur (S); R′ and R″ are each independently selected from the group consisting of C1-C6 alkyl or halogenated C1-C6 alkyl;
L is —NHSO₂—;
A is C1-C8 alkyl, wherein A is optionally substituted with one or more substituents independently selected from fluorine (—F), chlorine (—Cl), bromine (—Br);
X₁, X₂, X₃, and X₄ are each independently selected from the group consisting of —CR³—, or —N—;
R³ is selected from the group consisting of hydrogen (H), halogen, hydroxyl (—OH), amino (—NH₂), cyano (—CN), —C(═O)OH, NHCOR⁷, —(C═O)R⁸, —C(═O)OR⁹, C1-C6 alkyl, C1-C6 alkoxy, or C3-C8 heterocycle, wherein each occurrence of R³ is optionally substituted with one or more substituents independently selected from the group consisting of hydroxyl (—OH), amino (—NH₂), cyano (—CN), halogen, C1-C6 alkyl, halogenated C1-C6 alkyl, C3-C8 cycloalkyl, C1-C6 alkoxy, halogenated C1-C6 alkoxy, —C(═O)OR′, —C(═O)R′, —NHR′, or —NR′R″; R⁴ and R⁵ together may form a carbonyl (—C═O) functional group; R′ and R″ are each independently selected from the group consisting of C1-C6 alkyl or halogenated C1-C6 alkyl;
R⁷, R⁸, and R⁹ are each independently selected from NH₂, methyl, ethyl, propyl, or isopropyl;

9. According to claim 1, or a stereoisomer, tautomer, pharmaceutically acceptable salt, solvate, deuterated analog, metabolite, or prodrug thereof, what is claimed is a compound selected from the group consisting of:

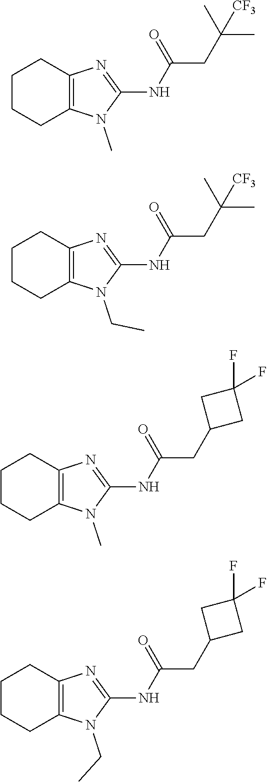 [SMILES: Cn1c(NC(=O)CC(C)(C)C(F)(F)F)nc2c1CCCC2 ; CCn1c(NC(=O)CC(C)(C)C(F)(F)F)nc2c1CCCC2 ; Cn1c(NC(=O)CC2CC(F)(F)C2)nc2c1CCCC2 ; CCn1c(NC(=O)CC2CC(F)(F)C2)nc2c1CCCC2]

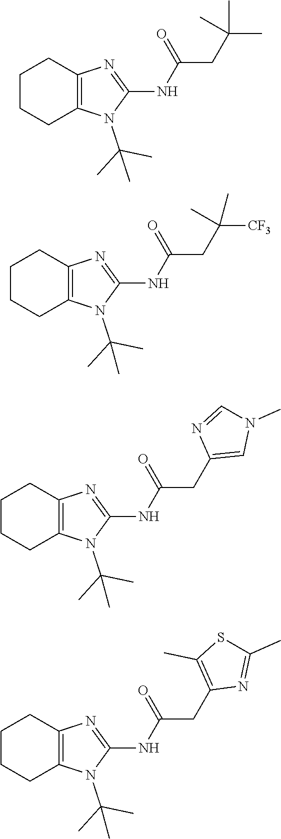 [SMILES: CC(C)(C)CC(=O)Nc1nc2c(n1C(C)(C)C)CCCC2 ; CC(C)(C)n1c(NC(=O)CC(C)(C)C(F)(F)F)nc2c1CCCC2 ; Cn1cnc(CC(=O)Nc2nc3c(n2C(C)(C)C)CCCC3)c1 ; Cc1nc(CC(=O)Nc2nc3c(n2C(C)(C)C)CCCC3)c(C)s1]

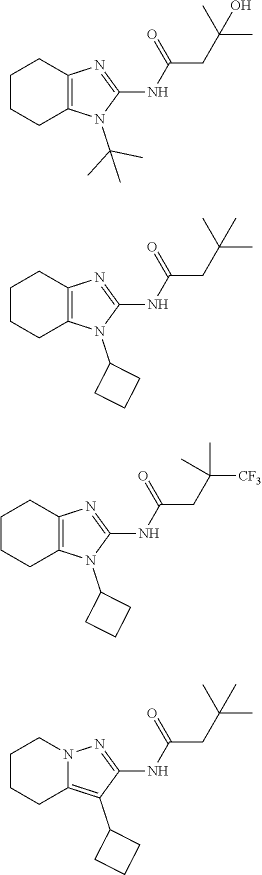 [SMILES: CC(C)(O)CC(=O)Nc1nc2c(n1C(C)(C)C)CCCC2 ; CC(C)(C)CC(=O)Nc1nc2c(n1C1CCC1)CCCC2 ; CC(C)(CC(=O)Nc1nc2c(n1C1CCC1)CCCC2)C(F)(F)F ; CC(C)(C)CC(=O)Nc1nn2c(c1C1CCC1)CCCC2]

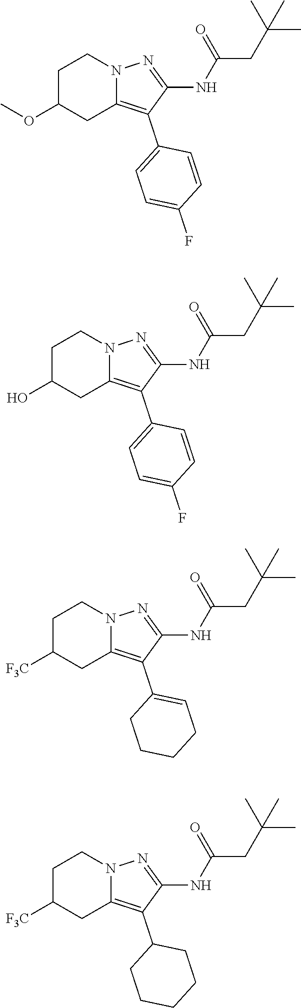 [SMILES: COC1CCn2nc(NC(=O)CC(C)(C)C)c(-c3ccc(F)cc3)c2C1 ; CC(C)(C)CC(=O)Nc1nn2c(c1-c1ccc(F)cc1)CC(O)CC2 ; CC(C)(C)CC(=O)Nc1nn2c(c1C1=CCCCC1)CC(C(F)(F)F)CC2 ; CC(C)(C)CC(=O)Nc1nn2c(c1C1CCCCC1)CC(C(F)(F)F)CC2]

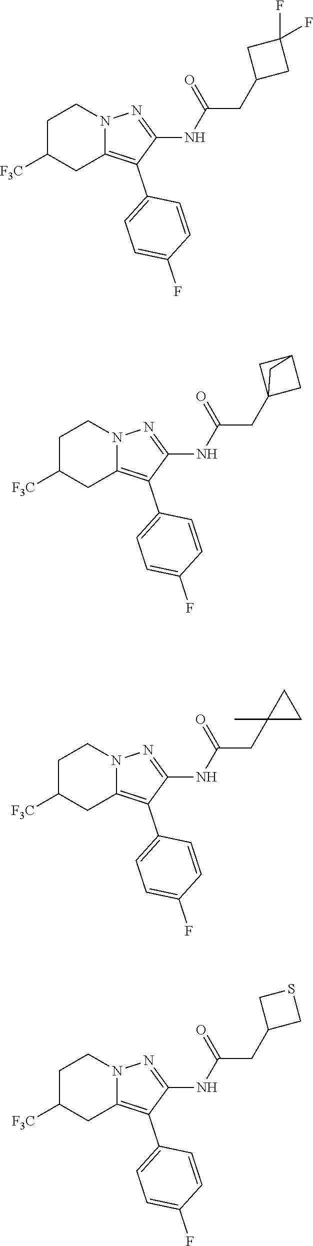 [SMILES: O=C(CC1CC(F)(F)C1)Nc1nn2c(c1-c1ccc(F)cc1)CC(C(F)(F)F)CC2 ; O=C(CC12CC(C1)C2)Nc1nn2c(c1-c1ccc(F)cc1)CC(C(F)(F)F)CC2 ; CC1(CC(=O)Nc2nn3c(c2-c2ccc(F)cc2)CC(C(F)(F)F)CC3)CC1 ; O=C(CC1CSC1)Nc1nn2c(c1-c1ccc(F)cc1)CC(C(F)(F)F)CC2]

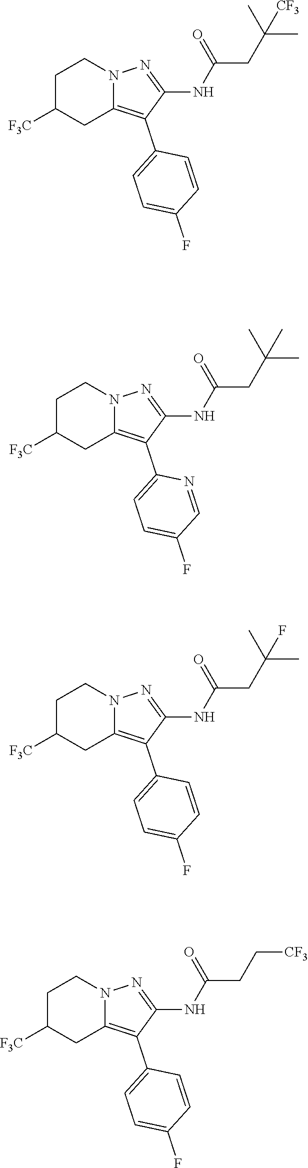 [SMILES: CC(C)(CC(=O)Nc1nn2c(c1-c1ccc(F)cc1)CC(C(F)(F)F)CC2)C(F)(F)F ; CC(C)(C)CC(=O)Nc1nn2c(c1-c1ccc(F)cn1)CC(C(F)(F)F)CC2 ; CC(C)(F)CC(=O)Nc1nn2c(c1-c1ccc(F)cc1)CC(C(F)(F)F)CC2 ; O=C(CCC(F)(F)F)Nc1nn2c(c1-c1ccc(F)cc1)CC(C(F)(F)F)CC2]

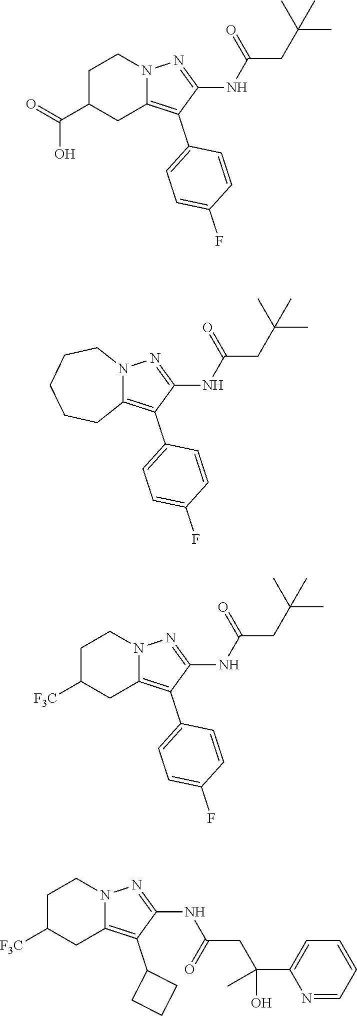 [SMILES: CC(C)(C)CC(=O)Nc1nn2c(c1-c1ccc(F)cc1)CC(C(=O)O)CC2 ; CC(C)(C)CC(=O)Nc1nn2c(c1-c1ccc(F)cc1)CCCCC2 ; CC(C)(C)CC(=O)Nc1nn2c(c1-c1ccc(F)cc1)CC(C(F)(F)F)CC2 ; CC(O)(CC(=O)Nc1nn2c(c1C1CCC1)CC(C(F)(F)F)CC2)c1ccccn1]

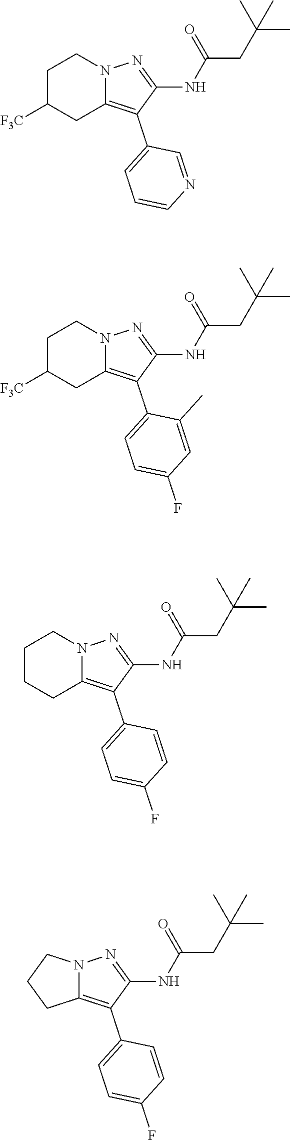 [SMILES: CC(C)(C)CC(=O)Nc1nn2c(c1-c1cccnc1)CC(C(F)(F)F)CC2 ; Cc1cc(F)ccc1-c1c(NC(=O)CC(C)(C)C)nn2c1CC(C(F)(F)F)CC2 ; CC(C)(C)CC(=O)Nc1nn2c(c1-c1ccc(F)cc1)CCCC2 ; CC(C)(C)CC(=O)Nc1nn2c(c1-c1ccc(F)cc1)CCC2]

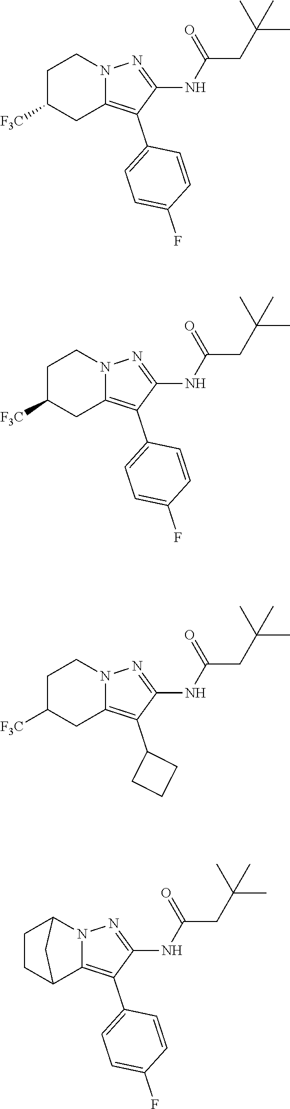 [SMILES: CC(C)(C)CC(=O)Nc1nn2c(c1-c1ccc(F)cc1)C[C@H](C(F)(F)F)CC2 ; CC(C)(C)CC(=O)Nc1nn2c(c1-c1ccc(F)cc1)C[C@@H](C(F)(F)F)CC2 ; CC(C)(C)CC(=O)Nc1nn2c(c1C1CCC1)CC(C(F)(F)F)CC2 ; CC(C)(C)CC(=O)Nc1nn2c(c1-c1ccc(F)cc1)C1CCC2C1]

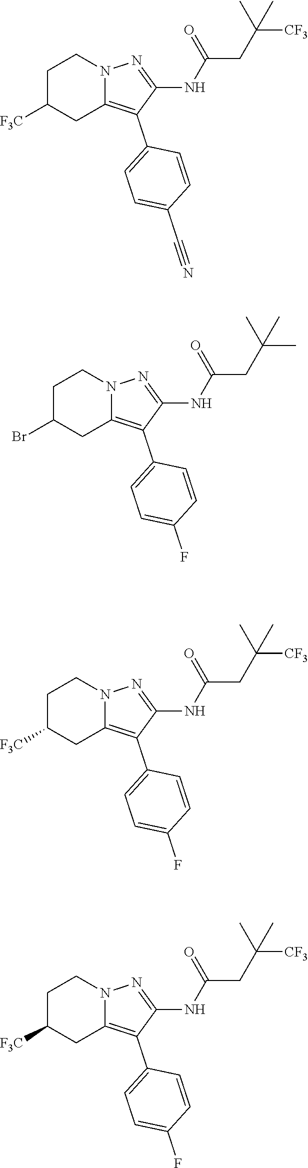 [SMILES: CC(C)(CC(=O)Nc1nn2c(c1-c1ccc(C#N)cc1)CC(C(F)(F)F)CC2)C(F)(F)F ; CC(C)(C)CC(=O)Nc1nn2c(c1-c1ccc(F)cc1)CC(Br)CC2 ; CC(C)(CC(=O)Nc1nn2c(c1-c1ccc(F)cc1)C[C@H](C(F)(F)F)CC2)C(F)(F)F ; CC(C)(CC(=O)Nc1nn2c(c1-c1ccc(F)cc1)C[C@@H](C(F)(F)F)CC2)C(F)(F)F]

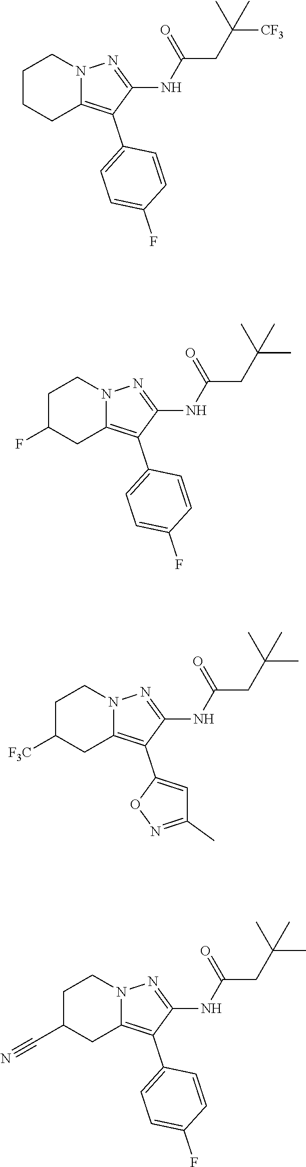 [SMILES: CC(C)(CC(=O)Nc1nn2c(c1-c1ccc(F)cc1)CCCC2)C(F)(F)F ; CC(C)(C)CC(=O)Nc1nn2c(c1-c1ccc(F)cc1)CC(F)CC2 ; Cc1cc(-c2c(NC(=O)CC(C)(C)C)nn3c2CC(C(F)(F)F)CC3)on1 ; CC(C)(C)CC(=O)Nc1nn2c(c1-c1ccc(F)cc1)CC(C#N)CC2]

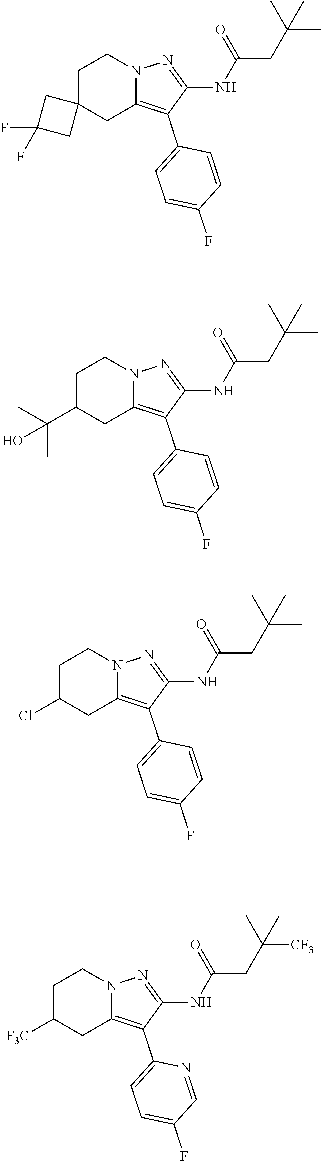

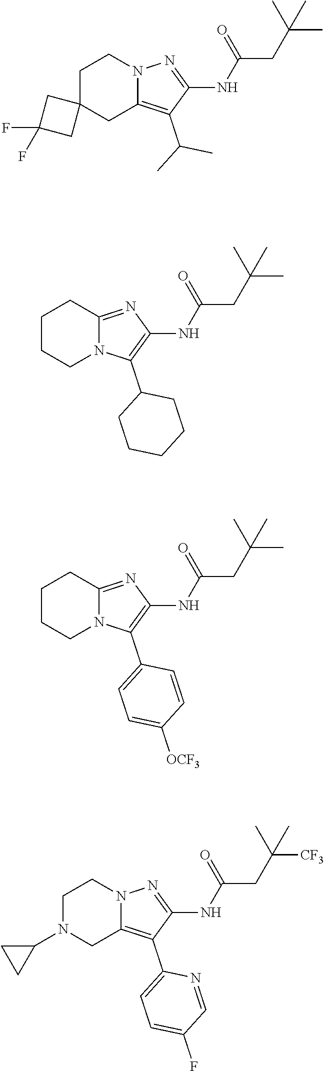 [SMILES: CC(C)c1c(NC(=O)CC(C)(C)C)nn2c1CC1(CC2)CC(F)(F)C1 ; CC(C)(C)CC(=O)Nc1nc2n(c1C1CCCCC1)CCCC2 ; CC(C)(C)CC(=O)Nc1nc2n(c1-c1ccc(OC(F)(F)F)cc1)CCCC2 ; CC(C)(CC(=O)Nc1nn2c(c1-c1ccc(F)cn1)CN(C1CC1)CC2)C(F)(F)F]

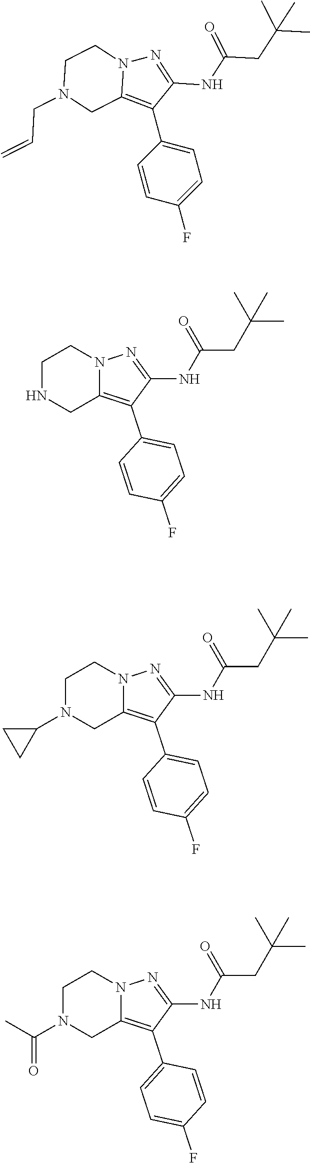

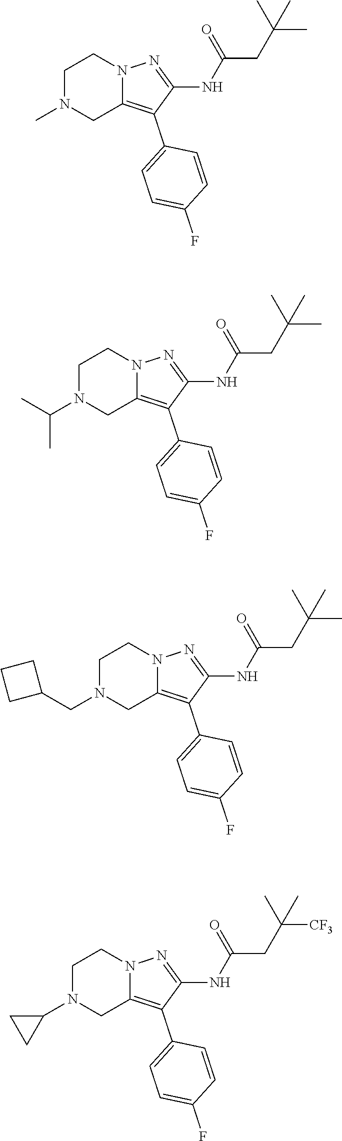

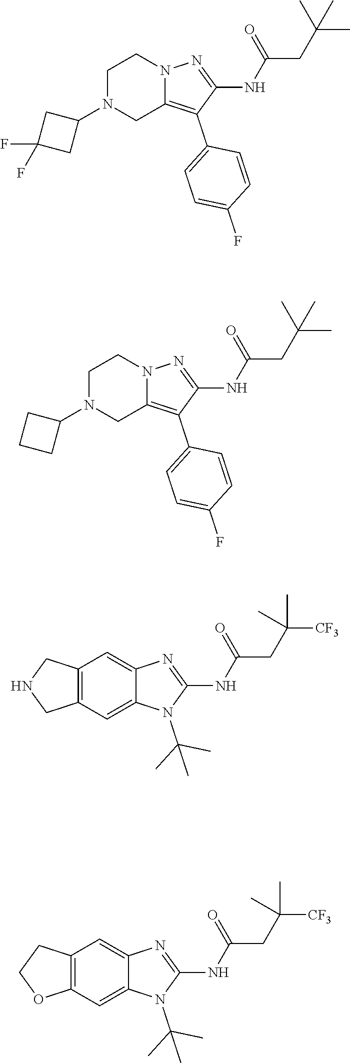 [SMILES: CC(C)(C)CC(=O)Nc1nn2c(c1-c1ccc(F)cc1)CN(C1CC(F)(F)C1)CC2 ; CC(C)(C)CC(=O)Nc1nn2c(c1-c1ccc(F)cc1)CN(C1CCC1)CC2 ; CC(C)(C)n1c(NC(=O)CC(C)(C)C(F)(F)F)nc2cc3c(cc21)CNC3 ; CC(C)(C)n1c(NC(=O)CC(C)(C)C(F)(F)F)nc2cc3c(cc21)OCC3]

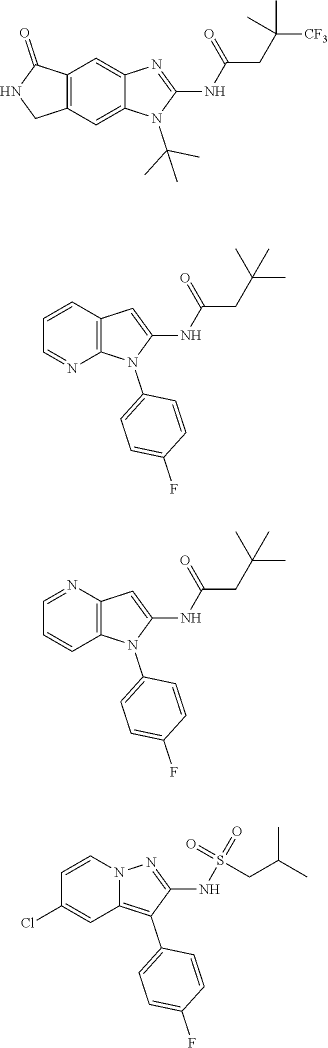 [SMILES: CC(C)(C)n1c(NC(=O)CC(C)(C)C(F)(F)F)nc2cc3c(cc21)CNC3=O ; CC(C)(C)CC(=O)Nc1cc2cccnc2n1-c1ccc(F)cc1 ; CC(C)(C)CC(=O)Nc1cc2ncccc2n1-c1ccc(F)cc1 ; CC(C)CS(=O)(=O)Nc1nn2ccc(Cl)cc2c1-c1ccc(F)cc1]

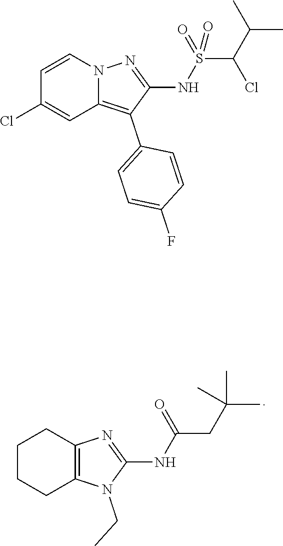 [SMILES: CC(C)C(Cl)S(=O)(=O)Nc1nn2ccc(Cl)cc2c1-c1ccc(F)cc1 ; CCn1c(NC(=O)CC(C)(C)C)nc2c1CCCC2]

10. A pharmaceutical composition comprising a compound as described in claim 1, or a pharmaceutically acceptable salt thereof, a tautomers, stereoisomers, hydrates, solvates, pharmaceutically acceptable salts, or prodrugs, along with a pharmaceutically acceptable excipient.

11. A method of preparing a Kv7 potassium channel activator comprising administering an effective amount of a compound of claim 1 to a mammal for treating and/or alleviating symptoms of related diseases: epilepsy, pain, migraine, depression, bipolar disorder, amyotrophic lateral sclerosis (ALS), neurodegenerative diseases and combinations thereof.

12. The method of claim 11, wherein the disorder is epilepsy, neuropathic pain, inflammatory pain, persistent pain, cancer pain, postoperative pain, migraine, depression, bipolar disorder, amyotrophic lateral sclerosis (ALS), or neurodegenerative diseases.
